(19) United States
(12) Patent Application Publication
(10) Pub. No.: US 2022/0220100 A1
(43) Pub. Date: Jul. 14, 2022

(54) PHOTOCHROMIC AND ELECTROCHROMIC DIARYLETHENE COMPOUNDS WITH IMPROVED PHOTOSTABILITY AND SOLUBILITY

(71) Applicant: SOLUTIA CANADA INC., Vancouver (CA)

(72) Inventors: SHAO-KAI CHEN, VANCOUVER (CA); RICHARD JOHN BURFORD, BURNABY (CA); AMIR MAHMOUD ASADIRAD, BURNABY (CA); TUOQI WU, BURNABY (CA); JAMES DANIEL SENIOR, SURREY (CA); NATALIE ELAINE CAMPBELL, VANCOUVER (CA); GLEN RAMSAY BREMNER, NEW WESTMINSTER (CA); NEIL ROBIN BRANDA, NORTH VANCOUVER (CA); JEREMY GRAHAM FINDEN, NORTH VANCOUVER (CA)

(73) Assignee: SWITCH MATERIALS INC., BURNABY, BC (CA)

(21) Appl. No.: 17/601,096

(22) PCT Filed: Apr. 2, 2020
(86) PCT No.: PCT/CA2020/050434

§ 371 Date: Oct. 4, 2021

Related U.S. Application Data
(60) Provisional application No. 62/828,945, filed on Apr. 3, 2019.

(51) Int. Cl. C07D 409/14; C07F 7/08; C07D 413/14

(52) U.S. Cl. CPC C07D 409/14; C07D 413/14; C07F 7/0814

(57) ABSTRACT

A diarylethene compound reversibly convertible under photochromic and electrochromic conditions between a ring-open isomer of Formula (1A) and a ring-closed isomer of Formula (IB) wherein R₅ is a substituted phenyl ring and Re is a substituted thiophene ring is provided. The photochromic-electrochromic diarylethene compound of Formula (1A)/(1B) have improved photochromic, electrochromic or photochromic and electrochromic properties, and is useful to provide variation of the light transmission properties of optical filters. The compound also possesses improved solubility making it suitable for incorporation in commercial products.

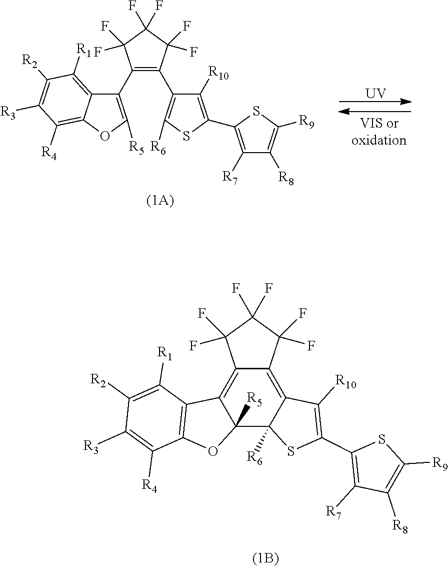

### FIELD

This invention relates to diarylethene compounds having electrochromic and photochromic properties. In particular, the invention relates to diarylethene compounds having improved photostability and solubility, compared to other diarylethene compounds.

### BACKGROUND

A variety of materials or systems with variable light transmitting qualities are known, including suspended particle displays or screens, electrochromic, photochromic and thermochromic materials, and those that are hybrid—having two or more of photo-, electro- or thermochromic qualities. The materials may be solid, liquid, gel or the like, the particular state and composition of the material being dependent upon, or limited by, the needs of the particular system. For example, the material may need to be conductive or insulative, may need to solubilize all components or only selected components of the system, and may further need to be tolerant of chemical transitions occurring within the material to achieve the desired light transmitting qualities.

Photochromic molecules are useful for a variety of research and commercial applications in fields ranging from sunglasses to memory storage devices. For example, optical filters are widely used to control visible light and solar energy. Optical filters have found a range of uses in vehicle and architectural glazings, as well as ophthalmic devices. A number of technologies have been developed to vary the degree of light transmittance using photochromic, thermochromic, electrochromic, liquid crystal and suspended particle display technologies, leading to a myriad of configurations seeking to obtain improvements in stability, control in switching, fatigue resistance, sensitivity and the like. Diarylethene compounds have found favour for several of these traits, and are the subject of continued investigation.

PCT Publication WO2004/015024 discloses compounds that are both photochromic and electrochromic, and methods of making such compounds, and describes a mechanism of catalytic electrochromism. Briefly, when a potential is applied to a switching material comprising a ring-closed isomer (II), the chromophore is oxidized to provide radical cation (II+). This radical cation undergoes ring-opening to provide radical cation (I+). As oxidation of the ring open (I) isomer requires a substantially greater potential, the radical cation (I+) oxidizes a neighbouring molecule and is neutralized to provide ring-open isomer (I). The potential required to oxidize the ring-closed and ring-open isomers may vary with chromophore structure. This interconversion between ring-open and ring-closed isomers is repeatable over many cycles. The neighbouring molecule oxidized to provide an electron to neutralize radical cation (I+) may be a chromophore in a ring-closed configuration or may be another neighbouring molecule. Where the oxidized neighbour molecule is a ring-closed chromophore, this contributes to the catalytic ring-opening effect that is advantageous of such systems, allowing transition of the switching material from a dark to a faded state with a less than stoichiometric injection of holes and electrons. PCT Publication WO2010/142019 describes variable transmittance optical filters comprising a material capable of transitioning between light and dark states in response to light and electric voltage, the material comprising a chromophore that has both electrochromic and photochromic properties.

Light transmission properties of such optical filters may be varied by selection of a photochromic-electrochromic diarylethene compound with greater or lesser light absorbance in the ring-open or ring-closed form. To provide for such variation, there is a need for molecules with improved photochromic, electrochromic or photochromic and electrochromic properties. Furthermore, there is a need for molecules with improved solubility properties for incorporation into commercial products.

Terthiophenes are critical building blocks for many functional organic materials. For purposes of synthetic introduction to a larger molecule, 3′-bromo-2,2′:5′,2″-terthiophene is of particular interest. This compound can be synthesized in one step and has been previously functionalized with a variety of substituents, typically in the 5 and 5″ positions. However, there remains a need for a synthetic route for substitution in the 4 and 4″ positions.

### SUMMARY

In one aspect, the present disclosure provides compounds according to Formula IA/IB, reversibly convertible under photochromic and electrochromic conditions between a ring-open isomer A and a ring-closed isomer B:

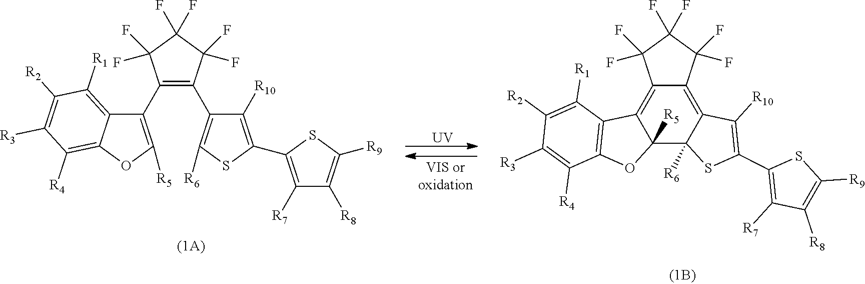

wherein:

each of R₁, R₂, R₃, and R₄ is independently: H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of O, S, N or Si, or —O—R, wherein R is a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

R₅ is

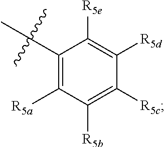

R₆ is

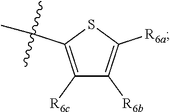

each of R₅ₐ, R_{5b}, R_{5c}, R_{5d}, and R₅ₑ is independently H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of 0, H, N or Si, or —R, wherein R is a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

each of R₆ₐ and R_{6b} is independently H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of O, S, N or Si, or R₆ₐ and R_{6b} are both —C(R₁₂)(R₁₃)— and joined by —(C(R₁₄)(R₁₅))ₙ— to form a 5-, 6- or 7-membered ring where n is 1, 2 or 3, respectively, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

R_{6c} is a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

each of R₇, R₈ and R₉ is independently H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of O, S, N or Si, or R₇ and R₈ or R₈ and R₉ are both —C(R₁₆)(R₁₇)— and joined by —(C(R₁₈)(R₁₉))ₙ— to form a 5-, 6- or 7-membered ring where n is 1, 2 or 3, respectively, wherein each or R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si; and

R₁₀ is H or a linear or branched, saturated or unsaturated, substituted or unsubstituted alkyl group with 1 to 20 carbons.

Various aspects of the present disclosure also provide compounds according to Formula IA/IB, reversibly convertible under photochromic and electrochromic conditions between a ring-open isomer A and a ring-closed isomer B:

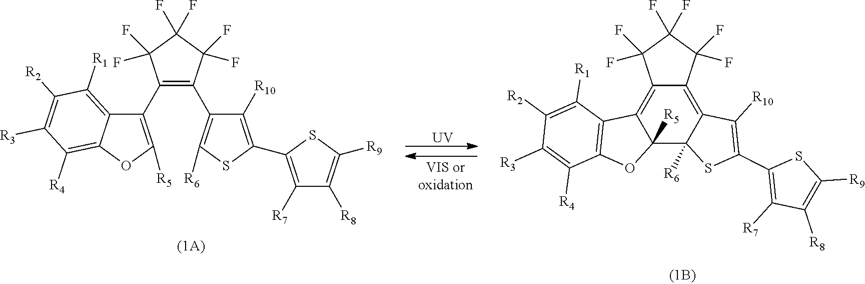

wherein:

each of R₁, R₂, R₃, and R₄ is independently H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of O, S, N or Si, or —O—R, wherein R is a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

R₅ is

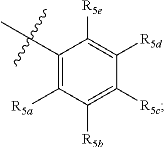

R₆ is

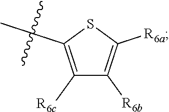

each of R₅ₐ, R_{6b}, R_{5c}, R_{5d}, and R₅ₑ is independently H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of O, S, N or Si, or —O—R, wherein R is a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

each of R₆ₐ, R_{6b} and R_{6c} is independently H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of O, S, N or Si, wherein R_{6b} is of equal or larger steric size than R₆ₐ or R₆ₐ and R_{6b} are both —C(R₁₂)(R₁₃)— and joined by —(C(R₁₄)(R₁₅))ₙ— to form a 5-, 6- or 7-membered ring where n is 1, 2 or 3, respectively, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

each of R₇, R₈ and R₉ is independently H, a linear or branched, saturated or unsaturated, substituted or unsubstituted alkyl group with 1 to 20 carbons, a linear or branched, saturated or unsaturated, substituted or unsubstituted heteroalkyl group with 1 to 20 carbons and comprising one or more of O, S, N or Si, or R₇ and R₈ or R₈ and R₉ are both —C(R₁₆)(R₁₇)— and joined by —(C(R₁₈)(R₁₉))ₙ— to form a 5-, 6- or 7-membered ring where n is 1, 2 or 3, respectively, wherein each or R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si; and

R₁₀ is H or a linear or branched, saturated or unsaturated, substituted or unsubstituted alkyl group with 1 to 20 carbons.

Various aspects of the present disclosure also provide a method of synthesizing a 4, 4″-substituted 2,2′:5′,2″-terthiophene, the method comprising reacting a terthiophene substrate with more than 2 molar equivalents of a Lewis acid and more than 2 molar equivalents of an electrophile at room temperature, wherein the terthiophene substrate is a polythiophene comprising more than two thiophenes, and wherein the 4,4″-substituted 2,2′:5′,2″-terthiophene is synthesized in excess of a 5,5″-substituted 2,2′-5′,5″-terthiophene.

Other aspects and features of the present invention will become apparent to those of ordinary skill in the art upon review of the following description of specific embodiments of the invention in conjunction with the accompanying claims.

### BRIEF DESCRIPTION OF THE DRAWINGS

In drawings which illustrate embodiments of the invention,

FIG. 1 shows the absorbance (y-axis) of a chromophore at various wavelengths of light (x-axis, in nm) for two light sources—365 nm and solar simulator (SS) in the presence or absence of a UV blocking film. Solid line—absorbance plot of the chromophore in a faded state (“faded”—solid line); A: Solar simulator with UV blocking film; B: Solar simulator without UV blocking film; and D: 365 nm light source without UV blocking film.

FIG. 2 shows a graphical representation of the amount of chromophore remaining and degradation products linearly interpolated to 0.5 MJ/m² at 340 nm of exposure energy for chromophores reversibly convertible between structural isomers (IA) and (IB).

FIG. 3 shows chromophore S364 and degradation products thereof.

FIG. 4 shows a crystal structure of the Minus-HF degradation product of chromophore S364 viewed in a perspective orientation.

FIG. 5 shows the blocking of the formation of the Minus-HF degradation by a substituent at R_{6c}.

FIG. 6 shows a graphical representation of the amount of chromophore remaining and degradation products linearly interpolated to 0.5 MJ/m² at 340 nm of exposure energy for chromophores according to approach 1.

FIG. 7 shows a comparison between chromophores S340 and S377 with respect to internal thiophene rotation.

FIG. 8 shows a graphical representation of the amount of chromophore remaining and degradation products linearly interpolated to 0.5 MJ/m² at 340 nm of exposure energy for chromophores according to approach 1 and approach 2.

FIG. 9 shows 3′-bromo-2,2′:5′,2″-terthiophene substituent positions labelled with IUPAC numbering.

FIG. 10 shows the ¹H NMR spectrum of 3′-bromo-5,5″-di-tert-butyl-2,2′:5′,2″-terthiophene in CDCl₃.

FIG. 11 shows the ¹H NMR spectrum of 3′-bromo-4,4″-di-tert-butyl-2,2′:5′,2″-terthiophene in CDCl₃.

### DETAILED DESCRIPTION

In the context of the present disclosure, various terms are used in accordance with what is understood to be the ordinary meaning of those terms.

In various embodiments, the disclosure provides compounds according to Formula IA/IB, reversibly convertible under photochromic and electrochromic conditions between a ring-open isomer A and a ring-closed isomer B:

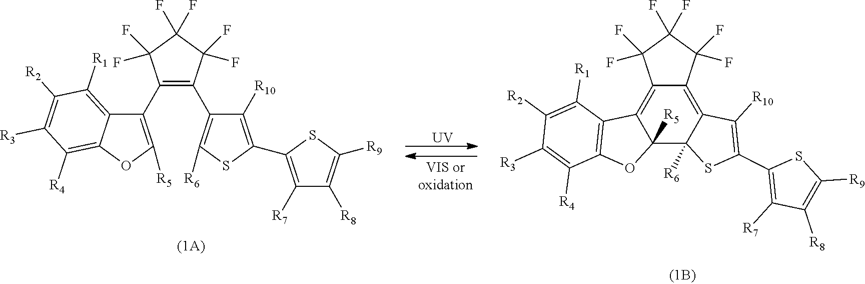

wherein:

each of R₁, R₂, R₃, and R₄ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si;

R₅ is

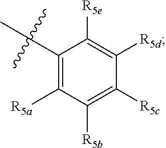

R₆ is

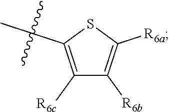

each of R₅ₐ, R_{5b}, R_{5c}, R_{5d}, and R₅ₑ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si; each of R₆ₐ and R_{6b} is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

R₆ₐ and R_{6b} are both —C(R₁₂)(R₁₃)— and joined by —(C(R₁₄)(R₁₅))ₙ— to form a 5-, 6- or 7-membered ring where n is 1, 2 or 3, respectively, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

R_{6c} is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si;

each of R₇, R₈ and R₉ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

R₇ and R₈ or R₈ and R₉ are both —C(R₁₆)(R₁₇)— and joined by —(C(R₁₈)(R₁₉))ₙ— to form a 5-, 6- or 7-membered ring where n is 1, 2 or 3, respectively, wherein each of R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si; and

R₁₀ is H or a linear or branched, saturated or unsaturated, substituted or unsubstituted alkyl group with 1 to 20 carbons.

In various embodiments, the disclosure also provides compounds according to Formula IA/IB, reversibly convertible under photochromic and electrochromic conditions between a ring-open isomer A and a ring-closed isomer B:

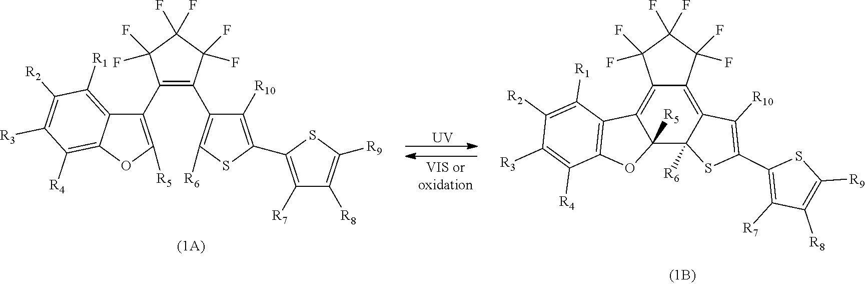

wherein:

each of R₁, R₂, R₃, and R₄ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si;

R₅ is

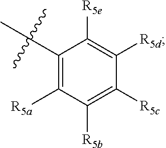

R₆ is

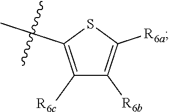

each of R₅ₐ, R_{5b}, R_{5c}, R_{5d}, and R₅ₑ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si; each of R₆ₐ, R_{6b} and R_{6c} is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si,

wherein R_{6b} is of equal or larger steric size than R₆ₐ, or

R₆ₐ and R_{6b} are both —C(R₁₂)(R₁₃)— and joined by —(C(R₁₄)(R₁₅))ₙ— to form a 5-, 6- or 7-membered ring where n is 1, 2 or 3, respectively, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

each of R₇, R₈ and R₉ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

R₇ and R₈ or R₈ and R₆ are both —C(R₁₆)(R₁₇)— and joined by —(C(R₁₈)(R₁₉))ₙ— to form a 5-, 6- or 7-membered ring where n is 1, 2 or 3, respectively, wherein each of R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si; and

R₁₀ is H or a linear or branched, saturated or unsaturated, substituted or unsubstituted alkyl group with 1 to 20 carbons.

These compounds surprisingly display improved photostability, together with increased solubility in organic solvents, compared to other diarylethene compounds. These properties result in the compounds disclosed herein being preferred for commercial product development.

Chromophores must meet a number of performance criteria based on their chemical structure and resulting physical properties to be suitable for commercial applications such as exterior glazing and optical filters. For example, the compounds need to have high absorbance at a photostationary state.

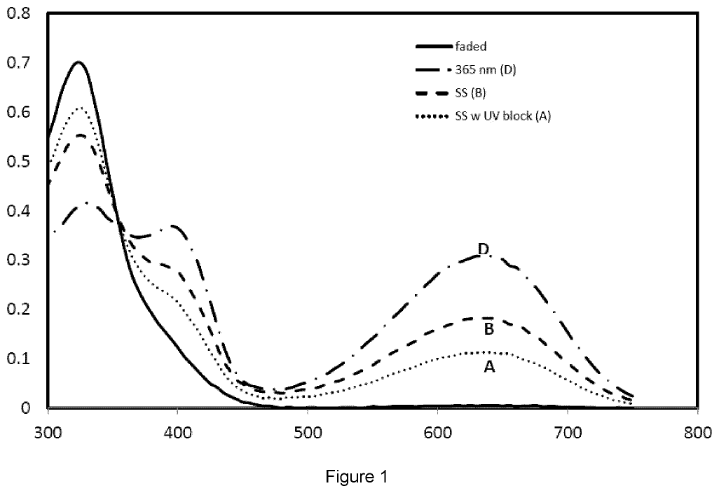

The term “photostationary state” (PSS) refers to an equilibrium state of a compound or material where the rate of the ring closing (forward) reaction is equal to the rate of the ring-opening reaction, when irradiated with light in a given region of the spectrum. In other words, the ratio of the ring-open isomer to the ring-closed isomer is at an equilibrium. PSS may be expressed with reference to a light source, or with reference to a type of light, for example, QUV, Xenon-arc lamp, Q-SUN, natural or filtered sunlight, UV, VIS, IR, NIR, full spectrum or the like, or with reference to a particular wavelength or range of wavelengths, or in the presence or absence of a filter. Some ring-open and ring-closed isomers may undergo isomerization from one to the other in response to different wavelengths of light. If a wavelength of light is used where only one of the isomers absorbs, irradiation results in complete isomerization to the other form. 254 nm, 313 nm or 365 nm light are commonly used in studies of UV-absorbing isomers, but this may not be representative of the PSS under other light conditions that include the visible spectrum such as natural or simulated sunlight (“full spectrum” light) and/or with filters that block a portion of the UV component of the light. For example, in a ring-closed (dark) state, the magnitude of the maximum absorbance in the visible range may change with the light source (FIG. 1). The wavelength at this peak in the visible range may be referred to as lambda max, or λₘₐₓ. Line D in FIG. 1 shows the absorption profile for a compound when exposed to a 365 nm light source. When full spectrum light from a solar simulator (Xenon arc lamp) is used as a light source (line B), a balance is achieved between the ring-closed (dark) state induced by the UV component, and a ring-open (faded) state induced by the visible component of the light. Inclusion of a UV blocking layer in the light path (line A) may reduce the UV component of the light, and the ring-opening reaction induced by the visible light component becomes more prominent. Different compounds may demonstrate different responsiveness to the composition of incident light. Where desired, the ratio of ring-open and ring-closed isomers at a PSS may be quantified by ¹H NMR spectroscopy, such as described in U.S. Pat. No. 7,777,055. In various embodiments, compounds with an increased absorbance at a photostationary state (PSS), or increased contrast ratio, are an improvement over other diarylethene compounds. A compound with a greater absorbance in the visible range can be used in lesser quantities in a formulation or material to achieve a desired contrast ratio, whereas a compound with a lower absorbance at a PSS may need a higher concentration to achieve a desired contrast ratio.

As used herein, “contrast ratio” is a ratio of the light transmittance of a material in the dark state and the light state. For example, a material may allow transmission of about 10% of the visible light (10% VLT) in a dark state, and about 60% of the visible light (60% VLT) in a faded or light state, providing a contrast ratio of 6:1. According to various embodiments of the invention, a material may have a contrast ratio of at least about 2 to about 20, or greater, or any amount or range therebetween, for example, about 2, 3, 4, 5, 6, 7, 8, 9, 10, 11, 12, 13, 14, 15, 16, 17, 18, 19 or 20. In some embodiments, a compound with a darker PSS (greater absorbance at lambda max) may provide a greater contrast ratio.

In various embodiments, at a PSS, some of the chromophore will be in the ring-closed isomer, with a small, but non-zero portion of ring-open isomer. The oxidation potential of a ring-open isomer may be more anodic than the oxidation potential of a ring-closed isomer. Exposure to a potential that is too far beyond what is necessary to oxidize the ring-closed isomer may result in oxidation of the ring-open isomer, which may be irreversible and result in electrochemically-induced degradation of the chromophore.

Thus, in various embodiments, an anodic chromophore may have an oxidation potential of from about 0.4 V to 1.2 V for the ring-closed isomer and about 1.0 V to 2.5 V for the ring-open isomer relative to an Ag/AgCl reference electrode. In various embodiments, the oxidation potential for the ring-open isomer is about 250 mV more anodic than the oxidation potential for the ring-closed isomer. For example, the oxidation potential may be about 260 mV, about 280 mV, about 300 mV, about 320 mV, about 340 mV, about 360 mV, about 380 mV, about 400 mV, about 420 mV, about 440 mV, or about 460 mV more anodic than the oxidation potential for the ring-closed isomer.

In various embodiments, the chromophore acts as an anodic species and a cathodic species is included in a switching material with the chromophore in order to balance the redox chemistry of the switching material. In various embodiments, the cathodic species is included in the switching material in a less than stoichiometric amount, relative to the amount of chromophore. The reduction potential of a cathodic species should be suitably compatible with the oxidation potential of the anodic chromophore. If a reduction potential of a cathodic species is too close to an oxidation potential of the anodic chromophore, a spontaneous electron transfer may occur, initiating a ring-opening oxidation of the chromophore without the application of electricity. By selecting a cathodic species that is stable in both oxidized and reduced forms, and with a reduction potential more negative than the anodic chromophore, oxidative fading of the chromophore or a switching material comprising the chromophore may be prevented in the absence of an applied voltage. In some embodiments the reduction potential of a cathodic species may be about 100 mV, about 200 mV, about 300 mV, about 400 mV, about 500 mV, about 600 mV, about 700 mV, about 800 mV, about 900 mV, about 1000 mV, about 1100 mV, about 1200 mV or more, less anodic than the ring-closed oxidation potentials of the one or more chromophores in the switching material. In various embodiments, the reduction potential of a cathodic species may be at least 400 mV less anodic than the ring-closed oxidation potentials of the one or more chromophores in the switching material. Suitable cathodic species are described in PCT Publication WO2013/044371.

Photostability (resistance to light-induced degradation) may be measured by the amount of time required for the compound, or a material comprising the compound, to degrade to a certain point under light exposure. The light exposure may be constant, or cyclic. The light transmittance or absorbance of the compound, or material comprising the compound, may be determined at both a light state and dark state prior to testing, to determine a contrast ratio. During testing, the contrast ratio may be monitored (periodically or continually), the compound or material may be determined to have failed when the contrast ratio falls outside, or below, a selected range, or when the contrast ratio decreases to a percentage of the original contrast ratio. Photostability may also be expressed with reference to a light source or with reference to a type of light. In terms of testing chromophores for application as an exterior glazing or optical filter, suitable chromophores in a solvent should exhibit less than 10% degradation at 0.5 MJ/m² of simulated sunlight exposure measured at 340 nm and a black panel temperature of 82° C.

The terms “switching voltage”, “switching potential” and “potential” refer to the electric potential required for a compound, or a material comprising the compound, to achieve a faded or light state. Switching voltage may further refer to the relationship between voltage and time to switch. To assess the switching voltage of a compound or a material comprising the compound, the material may be first darkened by exposure to a light source, followed by passing an electric current through the material at a defined voltage or voltage range, and assessing the time until a clear state, or a desired increase in light transmission, is achieved. Switching voltage may be expressed as a voltage or range of voltages, for example, about 2.5 volts, about 2.2 volts, or less than about 2 volts. In some embodiments, the compound or material comprising the compound has a switching potential of about 0.5 volts to about 5 volts, about 1 volt to about 2.5 volts, or any voltage or range of voltages therebetween.

The term “switching time” refers to the time necessary for a compound or material comprising the compound to transition from a dark state to a light (or faded or clear) state, or from a light (or faded or clear) state to a dark state, or to alter light transmittance by a defined amount.

In various embodiments comprising two or more chromophores, the chromophores may have different oxidation potentials for the ring-open isomers and/or ring-closed isomers. To allow for oxidation of ring-closed isomers, while avoiding oxidation of a ring-open isomer, combinations of chromophores with closely matched ring-closed oxidation potentials may be selected. In some embodiments, the ring-closed oxidation potential of a first chromophore may be within 0 to 200 mV of the ring-closed oxidation potential of a second chromophore. In some embodiments the ring-closed oxidation potentials of the first and second chromophores may be separated by about 0 to about 200 mV, or any amount or range therebetween, for example 10, 20, 30, 40, 50, 60, 70, 80, 90, 100, 110, 120, 130, 140, 150, 160, 170, 180 or 190 mV.

In various embodiments, it may be desirable for a switching material to provide a uniform transition from a dark state to a faded or light state. For such embodiments, the first and second chromophores may be selected to have ring-closed oxidation potentials the same, or very close together, so that both ring-closed isomers are oxidized at a substantially equal rate. In other embodiments, it may be desirable for a switching material to transition through one or more intermediate colour states, where the colouration of individual chromophore species is noticeable. By selecting a first chromophore with a ring-closed oxidation potential less than that of the second chromophore, the first chromophore may be fully transitioned to its faded state before the second chromophore, allowing the dark state coloration of the second chromophore to be more pronounced before completing the transition to a fully faded or light state.

In addition to electrochemical compatibility, chromophores should also have sufficient solubility and compatibility with a selected solvent component (one or more than one solvents combined to provide a solvent component), in both ring-open and ring-closed configurations. The solvent component of a switching material dissolves the formulation components and facilitates diffusion of the chromophore and cathodic species through the formulation and to and from the electrodes. The chromophore in all redox states may be soluble in the solvent to avoid precipitation, crystallization or passivation of the electrodes by insoluble material. The solvent is generally inert, and does not participate in any side reactions or undergo degradation with weathering. Suitable solvents may include a cyclic carbonate, a carbonate, an alkyl ether, an ester, a diester, or a lactone.

For example, the solvent component may comprise one or more of triglyme, tetraglyme, 1,2-propylene carbonate, ethylene carbonate, 1,2-butylene carbonate, 2,3-butylene carbonate, delta-valerolactone, 3-methyl-2-oxazolidone, tetramethylurea, butyrolactone, cyclopentanone, ethylene glycol phenyl ether, diethylene glycol monobutyl ether, diethyl succinate, dimethyl glutarate, diethylene glycol n-butyl ether acetate, diisobutyl adipate, dihexyl azelate, diethyl maleate, diisooctyl azelate, triethylene glycol monobutyl ether (butoxytriglycol), diisooctyl dodecanedioate, 2-(2-ethylhexyloxy)ethanol, glyceryl triacetate, tetramethylene sulfoxide, dibutyl adipate, 3-dodecylheptamethyltrisiloxane, diethyl sebacate, dibutyl itaconate, 1,4-butanediol, butyl sulfoxide, diethylene glycol, octyl octanoate, hexyl octanoate, diisodecyl adipate, diethylene glycol monoethyl ether acetate, 1,3/1,4-cyclohexanedimethanol, 1-decanol, 2-methylglutaronitrile, methyl palmitate, tri(propylene glycol) butyl ether, 1-dodecanol, tetradecane, diethylene glycol hexyl ether, dioctyl ether, methyl stearate, hexyl hexanoate, butyl diglyme, triisopentylamine, bis(2-ethylhexyl) sebacate, 1,5-dicyanopentane, diisobutyl fumarate, 2,2,4-trimethyl-1.3-pentanediol dibenzoate, poly(ethylene glycol) monolaurate, poly(ethylene glycol) monooleate, hexaethyldisiloxane, poly(ethylene glycol) dioleate, triethylene glycol di-2-ethyl butyrate, tributyrin, 1,2,3-propanetriyl ester, tetramethylene sulfone (sulfolane), polyethylene glycol dimethyl ether, bis(2-ethylhexyl) adipate, tetraethylene glycol, hexa-decamethylheptasiloxane, dioctyl terephthalate, bis[2-(2-butoxyethoxy)ethyl] adipate, triethylene glycol bis(2-ethylhexanoate), 1,3-propylene carbonate, triethylene glycol monomethyl ether (methoxytriglycol), triethylene glycol monoethyl ether (ethoxytriglycol), 18-crown-ether, 1,3-dimethylimidazolidinone, poly(ethylene glycol) bis(2-ethylhexanoate), 1,5-pentanediol, di(ethylene glycol) dibenzoate, 2-ethylhexyl-(s)-lactate, tripropylene glycol, dipropylene glycol, 2,2,4-trimethyl-1,3-pentanediol monoisobutyrate (“Texanol”), tri(propylene glycol) methyl ether, di(propylene glycol) dibenzoate, dipropylene glycol n-butyl ether, diethyl azelate, dimethyl adipate, diethyl adipate, poly(propylene glycol) dibenzoate, propylene glycol phenyl ether, poly(ethylene glycol) dibenzoate, 2-ethyl-1,3-hexanediol, propylene glycol diacetate, dimethylglutarate, diethyl-2-dimethyl glutarate, dimethyl-2-methyl glutarate (“Rhodiasolv IRIS”) or the like. In various embodiments, the solvent does not comprise water.

A compound with greater solubility allows for a formulation or material with a greater concentration of coloured molecule to be incorporated into a composition. This may allow for increasing the contrast ratio for a compound with a lesser absorbance at PSS. Examples of solubilizing groups for the compound include alkoxy, ether, ester or siloxy groups. In various embodiments, compounds may be soluble in the solvent component at room temperature at about 6 wt %, 7 wt %, 8 wt %, 9 wt %, 10 wt % or greater for testing to determine whether a compound has application as an exterior glazing or optical filter. If the solubility of a compound in the solvent component is too low, then insufficient darkening may be achieved in the ring-closed configuration, depending on conditions of use for the compound.

In addition to the above, chromophores should have acceptable colour for both the ring-opened and ring-closed isomers. For example, the ring-open isomer of the chromophore should have a Yellowness Index (YI) of less than about 10 and an absorbance at 400 nm, as measured for a 2×10⁻⁵ M solution in a 1 cm pathlength cuvette. For the ring-closed isomer of the chromophore, the lambda max should be between about 475 nm and 700 nm, tunable according to desired colour and number of chromophores in the composition.

In various embodiments, compounds according to Formula IA/IB, reversibly convertible under photochromic and electrochromic conditions between a ring-open isomer A and a ring-closed isomer B achieve these properties of high absorbance at PSS, solubility of 6 wt % or greater in solvent at room temperature, acceptable colour for both ring-closed and ring-open isomers, high photostability and acceptable oxidation potentials, compared to other diarylethene compounds:

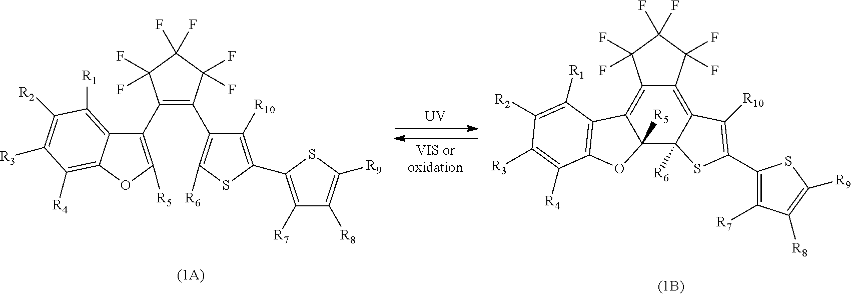

wherein:

each of R₁, R₂, R₃, and R₄ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si;

R₅ is

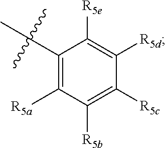

R₆ is

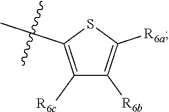

each of R₅ₐ, R_{5b}, R_{5c}, R_{5d}, and R₅ₑ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si;

each of R₆ₐ and R_{6b} is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

R₆ₐ and R_{6b} are both —C(R₁₂)(R₁₃)— and joined by —(C(R₁₄)(R₁₅))ₙ— to form a 5-, 6- or 7-membered ring where n is 1, 2 or 3, respectively, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

R_{6c} is

a linear or branched, saturated or unsaturated, substituted or unsubstituted alkyl group with 1 to 20 carbons, or

a linear or branched, saturated or unsaturated, substituted or unsubstituted heteroalkyl group with 1 to 20 carbons and comprising one or more of O, S, N or Si;

each of R₇, R₈ and R₉ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

R₇ and R₈ or R₈ and R₉ are both —C(R₁₆)(R₁₇)— and joined by —(C(R₁₈)(R₁₉))ₙ— to form a 5-, 6- or 7-membered ring where n is 1, 2 or 3, respectively, wherein each of R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si; and

R₁₀ is H or a linear or branched, saturated or unsaturated, substituted or unsubstituted alkyl group with 1 to 20 carbons.

In various embodiments, all linear or branched, saturated or unsaturated, substituted or unsubstituted alkyl groups and linear or branched, saturated or unsaturated, substituted or unsubstituted heteroalkyl groups referred to above have 1 to 10 carbons.

In various embodiments, R_{6c} is methyl, ethyl, propyl, butyl, pentyl or hexyl. In various embodiments, R_{6c} is methyl, ethyl or propyl. In various embodiments, R_{6c} is methyl.

In various embodiments, R_{6b} is H and R₆ₐ is methyl, ethyl, propyl or butyl. In various embodiments, R_{6b} is H and R₆ₐ is tert-butyl.

In various embodiments, R₆ₐ and R_{6b} are each —C(R₁₂)(R₁₃)— and joined by —(C(R₁₄)(R₁₅))₂— to form a 6-membered ring, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si. In various embodiments, each of R₁₂ and R₁₃ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si and R₁₄ and R₁₅ are H. In various embodiments, each of R₁₂ and R₁₃ is independently methyl, ethyl, propyl or butyl and R₁₄ and R₁₅ are H. In various embodiments, R₁₂ and R₁₃ are methyl and R₁₄ and R₁₅ are H.

In various embodiments, R₅ₐ, R_{6b}, R₅ₐ and R₅ₑ are H.

In various embodiments, R_{5c} is methyl, ethyl, propyl, butyl, pentyl or hexyl. In various embodiments, R_{5c} is tert-butyl.

In various embodiments, R₁₀ is H.

In various embodiments, R₇ and R₈ are H and R₉ is methyl, ethyl, propyl, butyl, pentyl or hexyl. In various embodiments, R₇ and R₈ are H and R₉ is tert-butyl.

In various embodiments, R₇ is methyl, ethyl, propyl or butyl, R₈ is H and R₉ is methyl, ethyl, propyl, butyl, pentyl or hexyl. In various embodiments, R₇ is methyl, R₈ is H and R₉ is tert-butyl.

In various embodiments, R₈ and R₉ are each —C(R₁₆)(R₁₇)— and joined by —(C(R₁₈)(R₁₉))₂— to form a 6-membered ring, wherein each of R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si. In various embodiments, each of R₁₆ and R₁₇ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si and R₁₈ and R₁₉ are H. In various embodiments, each of R₁₆ and R₁₇ is independently methyl, ethyl, propyl or butyl and R₁₈ and R₁₉ are H. In various embodiments, R₁₆ and R₁₇ are methyl and R₁₈ and R₁₉ are H.

In various embodiments, R₁ and R₄ are H and R₂ and R₃ are —O—R, where R is a linear or branched, saturated or unsaturated, substituted or unsubstituted heteroalkyl with 1 to 8 carbons and comprising O, N or S. In various embodiments, R is a linear or branched, saturated or unsaturated, substituted or unsubstituted heteroalkyl with 1 to 8 carbons and comprising O. In various embodiments, R₁ and R₄ are H, and R₂ and R₃ are —OCH₃, —O(CH₂)₃CO₂CH₂CH₃, —OCH₂CH₂OCH₃, —OCH₂CH₂CH₂OCH₃, —O(CH₂CH₂O)₃CH₃, —OC(O)CH₃,

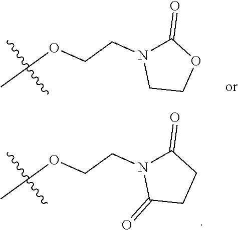

In various embodiments, R₁ and R₄ are H, and R₂ and R₃ are —OCH₃ or —O(CH₂)₃CO₂CH₂CH₃.

These properties are also achieved with compounds according to Formula IA/IB, reversibly convertible under photochromic and electrochromic conditions between a ring-open isomer A and a ring-closed isomer B:

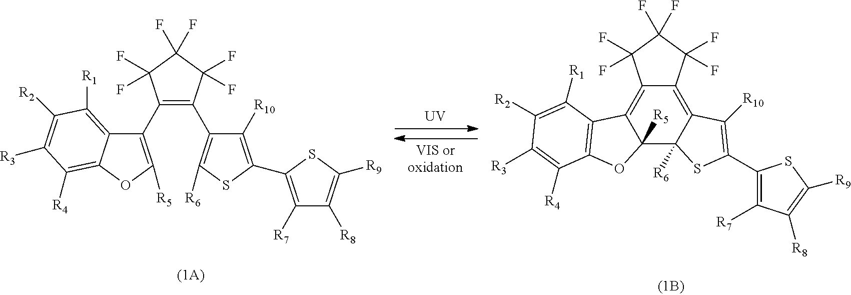

wherein:

each of R₁, R₂, R₃, and R₄ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si;

R₅ is

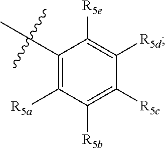

R₆ is

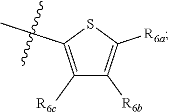

each of R₅ₐ, R_{6b}, R_{5c}, R_{5d}, and R₅ₑ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si; each of R₆ₐ, R_{6b} and R_{6c} is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si,

wherein R_{6b} is of equal or larger steric size than Rea, or

R₆ₐ and R_{6b} are both —C(R₁₂)(R₁₃)— and joined by —(C(R₁₄)(R₁₅))ₙ— to form a 5-, 6- or 7-membered ring where n is 1, 2 or 3, respectively, wherein each or R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;

each of R₇, R₈ and R₉ is independently:

H,

a linear or branched, saturated or unsaturated, substituted or unsubstituted alkyl group with 1 to 20 carbons,

a linear or branched, saturated or unsaturated, substituted or unsubstituted heteroalkyl group with 1 to 20 carbons and comprising one or more of O, S, N or Si, or

R₇ and R₈ or R₈ and R₉ are both —C(R₁₆)(R₁₇)— and joined by —(C(R₁₈)(R₁₉))ₙ— to form a 5-, 6- or 7-membered ring where n is 1, 2 or 3, respectively, wherein each or R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si; and

R₁₀ is H or a linear or branched, saturated or unsaturated, substituted or unsubstituted alkyl group with 1 to 20 carbons.

In various embodiments, all linear or branched, saturated or unsaturated, substituted or unsubstituted alkyl groups and linear or branched, saturated or unsaturated, substituted or unsubstituted heteroalkyl groups referred to above have 1 to 10 carbons.

In various embodiments, R_{6b} is not H.

In various embodiments, R₆ₐ and R_{6c} are H and R_{6b} is methyl, ethyl, propyl, butyl, pentyl or hexyl. In various embodiments, R₆ₐ and R_{6c} are H and R_{6b} is tert-butyl.

In various embodiments, R₆ₐ and R_{6b} are each —C(R₁₂)(R₁₃)— and joined by —(C(R₁₄)(R₁₅))₂— to form a 6-membered ring, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si. In various embodiments, each of R₁₂ and R₁₃ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si and R₁₄ and R₁₅ are H. In various embodiments, each of R₁₂ and R₁₃ is independently methyl, ethyl, propyl or butyl and R₁₄ and R₁₅ are H. In various embodiments, R₁₂ and R₁₃ are methyl and R₁₄ and R₁₅ are H.

In various embodiments, R₅ₐ, R_{6b}, R₅ₐ and R₅ₑ are H and R_{5c} is methyl, ethyl, propyl, butyl, pentyl or hexyl. In various embodiments, R₅ₐ, R_{6b}, R₅ₐ and R₅ₑ are H and R_{5c} is tert-butyl.

In various embodiments, R₁ and R₄ are H and R₂ and R₃ are independently a linear or branched, saturated or unsaturated, substituted or unsubstituted heteroalkyl group with 1 to 8 carbons and comprising one or more of O or N. In various embodiments, R₁ and R₄ are H and R₂ and R₃ are independently —O—R, where R is a linear or branched, saturated or unsaturated, substituted or unsubstituted heteroalkyl with 1 to 8 carbons and comprising O or N. In various embodiments, R₁ and R₄ are H and R₂ and R₃ are independently —O—R, where R is a linear or branched, saturated or unsaturated, substituted or unsubstituted heteroalkyl with 1 to 12 carbons and comprising O, N or S. In various embodiments, R is a linear or branched, saturated or unsaturated, substituted or unsubstituted heteroalkyl with 1 to 8 carbons and comprising O. In various embodiments, R₁ and R₄ are H, and R₂ and R₃ are —OCH₃, —O(CH₂)₃CO₂CH₂CH₃, —OCH₂CH₂OCH₃, —OCH₂CH₂CH₂OCH₃, —O(CH₂CH₂O)₃CH₃, —OC(O)CH₃,

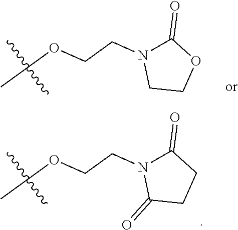

In various embodiments, R₇ and R₉ are H and R₈ is methyl, ethyl, propyl, butyl, pentyl or hexyl. In various embodiments, R₇ and R₉ are H and R₈ is tert-butyl.

In various embodiments, R₈ and R₉ are each —C(R₁₆)(R₁₇)— and joined by —(C(R₁₈)(R₁₉))₂— to form a 6-membered ring, wherein each of R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si. In various embodiments, each of R₁₆ and R₁₇ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si and R₁₈ and R₁₉ are H. In various embodiments, each of R₁₆ and R₁₇ is independently methyl, ethyl, propyl or butyl and R₁₈ and R₁₉ are H. In various embodiments, R₁₆ and R₁₇ are methyl and R₁₈ and R₁₉ are H.

The term “alkyl” refers to any linear or branched, non-aromatic monocyclic or polycyclic, substituted or unsubstituted alkyl group of from 1 to 20 carbons, for example, 2, 3, 4, 5, 6, 7, 8, 9, 10, 11, 12, 13, 14, 15, 16, 17, 18 or 19 carbons. Examples of alkyl groups include methyl, ethyl, propyl, isopropyl, butyl, iso-butyl, sec-butyl, tert-butyl, pentyl, 1-pentyl, iso-pentyl, neo-pentyl, hexyl, cyclopropane, cyclobutane, cyclopentane, cyclohexane or the like. The alkyl group may have one or more saturated or unsaturated bonds. In various embodiments, reference to a particular alkyl group includes all isomers thereof. For example, reference to butyl includes all isomers thereof, i.e. iso-butyl, sec-butyl and tert-butyl.

The term “heteroalkyl” refers to an alkyl group as defined above and comprising one or more heteroatoms such as Si, N, O or S as part of the alkyl group. Examples of cyclic heteroalkyl groups include aziridine, oxirane, thirane, oxaziridine, dioxirane, azetidine, oxetane, thietane, diazetidine, dioxetane, dithietane, azirine, oxirene, thirene, azete, oxete, thiete, dioxete, dithiete, pyrrolidine, oxolane, thiolane, borolane, silolane, dithiolane, dioxolane, oxazolidine, piperidine, oxane, thiane, piperazine, morpholine or the like. An alkyl group with an Si heteroatom may be described as a “silyl” or “silane” group.

The term “alkoxy” refers to any —O—R group, where R (and R′ for an ether as described below) may independently be H, alkyl, siloxy or aryl. Examples of alkoxy groups include those with from 1 to 20 carbons in a linear or branched chain, for example, methoxy or ethoxy, or longer alkyl groups. Other alkoxy groups include ethers (—R—O—R′—), alcohol (—OH) or alkoxide (—R—O-metal) or the like. An alkyl group comprising an alkoxy substituent group may be referred to as an “alkylalkoxy” group.

The term “carbonyl” refers to any group comprising RR′C═O, where R and R′ may be any group. Examples of carbonyl groups include aldehyde (—COH), ketone (COR′), ester (COOR′), acyl (RR′C═O), carboxyl, thioester (COSR′), primary amide (CONH₂), secondary amide (CONHR′) tertiary amide (CONRR′) or the like.

The term “steric size” refers to the relative three-dimensional spatial demand of a substituent.

In various embodiments, the compounds disclosed herein have increased photostability, together with an increased solubility compared to other diarylethene compounds, making the compounds disclosed herein more suitable for preparation as a product, material or device.

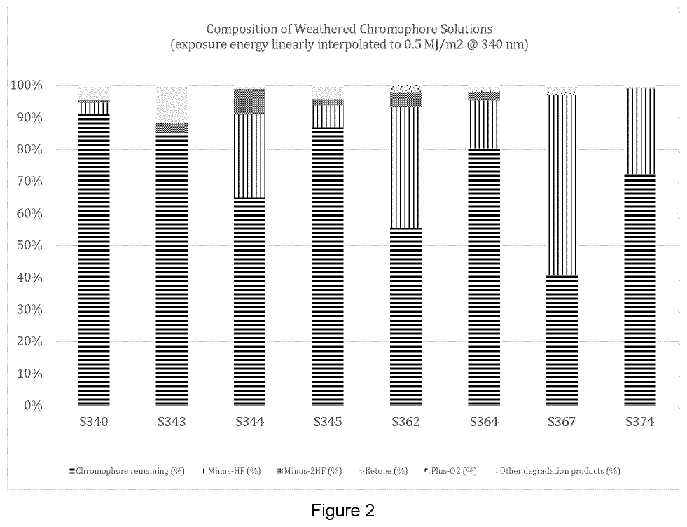

A first series of chromophores reversibly convertible between structural isomers (1A) and (1B) were synthesized and subject to screening tests for photostationary state, colour of the ring-open isomer, colour of the ring-closed isomer, solubility in organic solvent, and photostability. The results for each chromophore tested were compared to the results obtain for chromophore S340. These results are shown in Tables 1 and 2 and in FIG. 2.

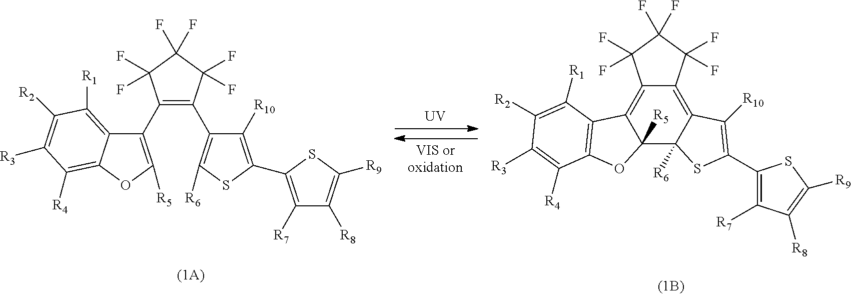

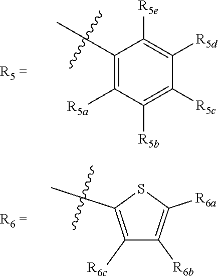

TABLE 1
Data for chromophores reversibly convertible between structural
isomers (1A) and (1B) for absorbance by the ring-open
isomer at 400 nm,
colour, solubility, photostationary state, and oxidation potential

| | | | | | Solubility | | |
|---|---|---|---|---|---|---|---|
| | | | | | in | | |
| | Abs @ | | | | Rhodia- | | |
| Chromo- | 400 | | λₘₐₓ | PSS | solv | RC | RO |
| phore | nm, RO¹ | YI² | (nm) | (SS) | IRIS | [O]³ | [O]⁴ |
| S340 | 0.128 | 5.61 | 591 | 0.417 | 7% | 0.95 | 1.35 |
| S343 | 0.11 | 4.87 | 573 | 0.315 | <1% | 1.1 | 1.37 |
| S344 | 0.1 | 2.91 | 583 | 0.337 | <3% | 1.05 | 1.36 |
| S345 | 0.0809 | 3.32 | 575 | 0.255 | <3% | 1 | 1.38 |
| S362 | 0.088 | 2.79 | 583 | 0.353 | >10% | 1.06 | 1.33 |
| S364 | 0.105 | 4.21 | 593 | 0.427 | >10% | 0.93 | 1.32 |
| S367 | 0.102 | 5.47 | 583 | 0.32 | >10% | 1.12 | 1.38 |
| S374 | 0.144 | 6.76 | 590 | 0.422 | >10% | 0.93 | 1.35 |

¹Absorption of ring-open isomer at 400 nm
²Yellowness Index
³oxidation potential of ring-closed isomer
⁴oxidation potential of ring-open isomer

TABLE 2
Data for chromophores reversibly convertible between structural
isomers (1A) and (1B) for photostability

| | | | | | | | | | Chromophore degradation | |
|---|---|---|---|---|---|---|---|---|---|---|
| | | | | | | | | | relative to S340 (interpolated | |
| | | | | | | | | | to 0.5 MJ/m² of energy | |
| | | | | | | | | | exposure at 340 nm) | |
| | | | | | | | | | Sum of | |
| | | | | | | | | | degradation | |
| | | | | | | | | | product | |
| | | | | | | | | | percentages | |
| | | | Percentages of chromophore remaining and degradation products | | | | | | relative to | |
| | | Exposure | linearly interpolated to 0.5 MJ/m2 @ 340 nm of exposure energy | | | | | | the sum of | Ratio of |
| | | energy | | | | | | Other | degradation | chromophore |
| | Weathering | (MJ/m2 @ | Chromophore | | | | | degradation | product | remaining |
| Chromophore | time | 340 nm, | Remaining | Minus-HF | Minus-2HF | Ketone | Sulfone | products | percentages | relative to |
| (wt %) | (h) | 82° C. bp) | (%) | (%) | (%) | (%) | (%) | (%) | for S340 | S340 |
| S340 (3%) | 1533 | 3.75 | 91.1 | 3.5 | 1.0 | 0.0 | 0.0 | 4.4 | 1.00 | 1.00 |
| S343 (3%) | 970 | 2.37 | 85.2 | 0.0 | 3.1 | 0.0 | 0.0 | 11.8 | 1.67 | 0.93 |
| S344 (3%) | 349 | 0.85 | 65.1 | 25.7 | 8.0 | 0.0 | 0.0 | 1.2 | 3.93 | 0.71 |
| S345 (3%) | 970 | 2.37 | 86.9 | 6.9 | 1.9 | 0.0 | 0.0 | 4.4 | 1.48 | 0.95 |
| S362 (10%) | 157 | 0.38 | 55.5 | 37.6 | 4.8 | 2.1 | 0.0 | 0.0 | 5.01 | 0.61 |
| S364 (10%) | 550 | 1.35 | 80.4 | 14.9 | 2.7 | 0.6 | 0.0 | 1.4 | 2.21 | 0.88 |
| S367 (10%) | 207 | 0.51 | 40.8 | 56.0 | 0.0 | 1.1 | 0.0 | 2.1 | 6.67 | 0.45 |
| S374 (10%) | 299 | 0.73 | 72.4 | 26.5 | 0.0 | 0.0 | 0.0 | 1.1 | 3.11 | 0.79 |

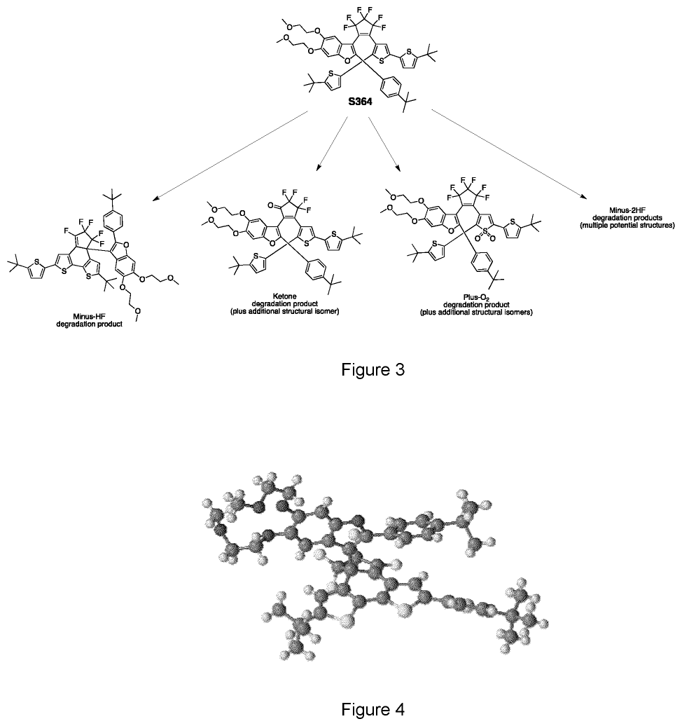

From examination of the data in Tables 1 and 2 and in FIG. 1, none of the chromophores tested had sufficient solubility and photostability. It is also evident from the weathering experiments that the chromophores tested, each with different substitution patterns, undergo some common degradation mechanisms, but to different extents based on their structure. Degradation products were identified based on mass spectrum data and mass difference from the molecular weight of the chromophore structure. No structure assignment is proposed for degradation products denoted as “other degradation products” based on available analytical data. The structure for the Minus-HF degradation product was determined by X-ray crystallography for S364 and S367. The degradation products listed in FIG. 2 are depicted in FIG. 3 using S364 for illustrative purposes.

Shown in Scheme 3a is the desired photocyclization reaction and conversion of the ring-open isomer of the chromophore to the desired ring-closed isomer. The carbon atoms undergoing reaction are identified in bold. The proposed degradation mechanism leading to the formation of the Minus-HF degradation product involves a photocyclization reaction as the first step of a two-step process, where a different set of carbon atoms (highlighted in bold) are involved as depicted in Scheme 3b. The second step of this process involves irreversible elimination of HF.

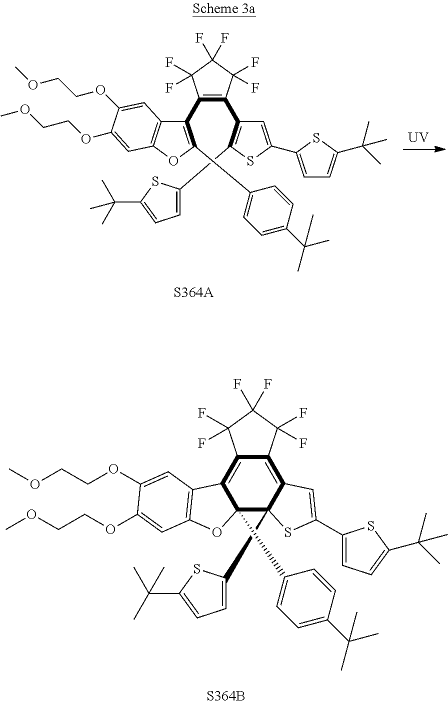

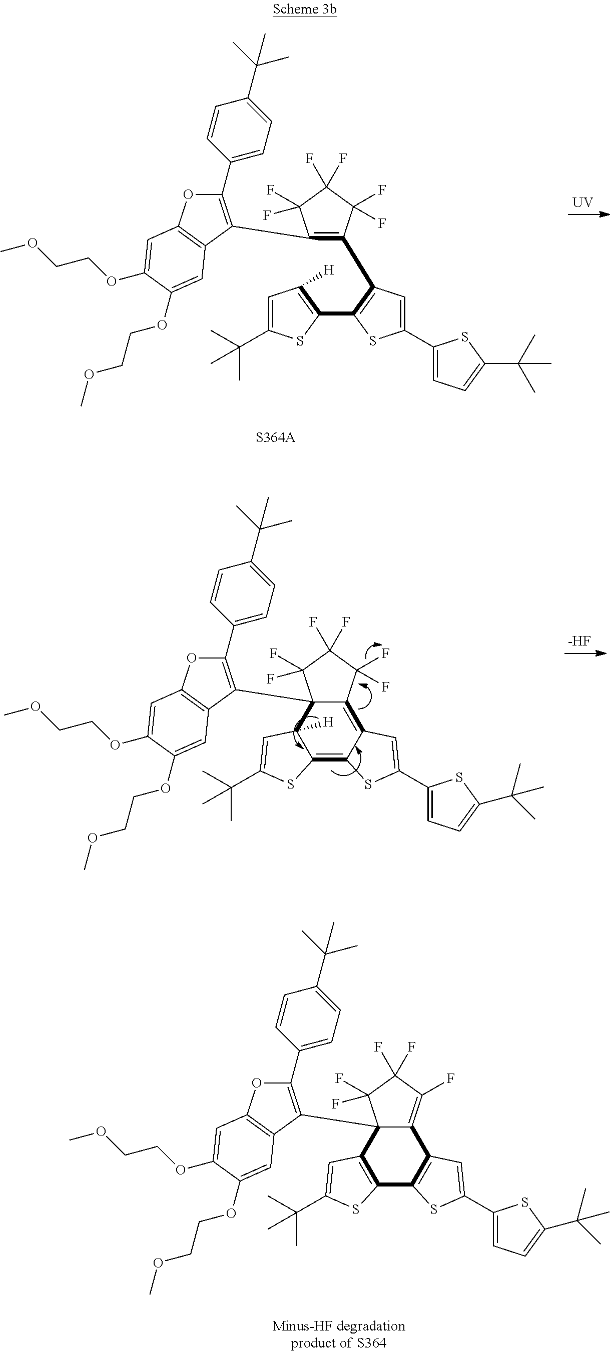

FIG. 4 shows the crystal structure of the Minus-HF degradation product of S364 viewed in a perspective orientation.

The stability of chromophores having a 5,5″-di-t-butylterthiophenyl moiety (S340, S374, S364, S362, S345, S344, S343 and S367) is greatly influenced by substituents on the benzofuran moiety.

Using S340 as a reference chromophore and decreasing electron density on the benzofuran group resulted in decreased stability, mainly due to an increased proportion of the Minus-HF degradation product. Chromophores S367, S343, S345 and S344 all have less electron density on the benzofuran ring (zero or one methoxy group) and show more rapid degradation compared to S340 (two methoxy groups).

Again using S340 as a reference chromophore and modifying the alkoxy substituent (i.e. methoxy versus longer alkoxy chain) results in decreased stability, mainly due to an increased proportion of the Minus-HF degradation product (S374 and S364 as compared to S340).

When designing the molecular structure of chromophores for exterior glazing applications or optical filters, it is desirable to modify the molecular structure for improved solubility in a specific solvent or solvent combination without compromising the photostability of the chromophore. For example, the solubility of S340, which has acceptable photostability, is improved by either replacing the methoxy groups on the benzofuran group with longer ethylene glycol chains (S364), acetate groups (S367) or long-chain ester groups (S374) but at the expense of photostability (increased Minus-HF degradation product).

Various embodiments disclosed herein provide a method to overcome the unacceptable weathering of chromophores as described above by strategically designing their molecular structures according to one of two different strategies in such a way that attachment of different solubilizing chains has minimal impact on photostability. These strategies provide different approaches to eliminate or reduce the amount of Minus-HF degradation product and result in chromophores with improved photostability and solubility.

Approach 1

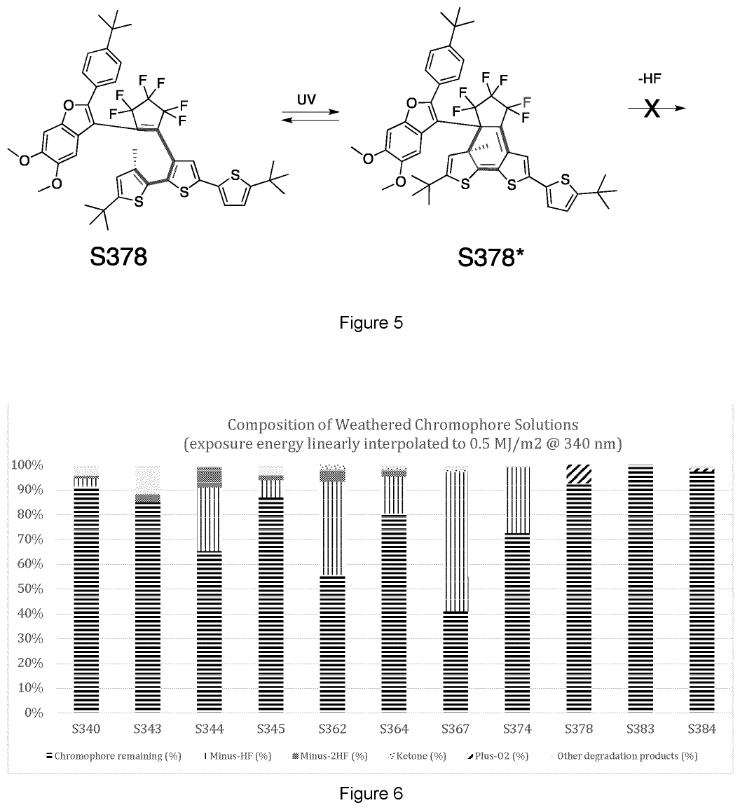

Stability of chromophores substituted in the 5 and 5″ positions of the terthiophene moiety is improved by replacing the proton in the 3-position (substituent R_{6c}) with an alkyl group or a heteroalkyl group, such as, for example, a methyl group (see, for example, chromophores S378, S383 and S384 in Table 3). Formation of the Minus-HF degradation product does not occur because the eliminated proton is replaced with an alkyl group. Referring to FIG. 5, if photocyclization of S378 to S378* were to occur, subsequent loss of HF as shown in Scheme 3b is not possible. In this case, replacement of the methoxy groups of S378 with longer chain ester groups as shown in S383 resulted in improved solubility, but not at the expense of photostability as was the case for the same structural modification going from S340 to S374. These data are summarized in Tables 3 and 4 and in FIG. 6.

TABLE 3
Data for chromophores according to approach 1 for absorbance of
ring-open isomer at 400 nm, colour, photostationary state,
solubility, and oxidation potential

| | | | | | Solubility | | |
|---|---|---|---|---|---|---|---|
| | | | | | in | | |
| | Abs @ | | | PSS | Rhodia- | | |
| Chromo- | 400 | | λₘₐₓ | (Steady | solv | RC | RO |
| phore | nm, RO¹ | YI² | (nm) | State) | IRIS | [O]³ | [O]⁴ |
| S340 | 0.128 | 5.61 | 591 | 0.417 | 7% | 0.95 | 1.35 |
| S343 | 0.11 | 4.87 | 573 | 0.315 | <1% | 1.1 | 1.37 |
| S344 | 0.1 | 2.91 | 583 | 0.337 | <3% | 1.05 | 1.36 |
| S345 | 0.0809 | 3.32 | 575 | 0.255 | <3% | 1 | 1.38 |
| S362 | 0.088 | 2.79 | 583 | 0.353 | >10% | 1.06 | 1.33 |
| S364 | 0.105 | 4.21 | 593 | 0.427 | >10% | 0.93 | 1.32 |
| S367 | 0.102 | 5.47 | 583 | 0.32 | >10% | 1.12 | 1.38 |
| S374 | 0.144 | 6.76 | 590 | 0.422 | >10% | 0.93 | 1.35 |
| S378 | 0.127 | 4.94 | 593 | 0.351 | <5% | 0.9 | 1.36 |
| S383 | 0.144 | 4.52 | 590 | 0.408 | >10% | 0.91 | 1.36 |
| S384 | 0.107 | 4.13 | 593 | 0.342 | >10% | 0.87 | 1.31 |

¹Absorption of ring-open isomer at 400 nm
²Yellowness Index
³oxidation potential of ring-closed isomer
⁴oxidation potential of ring-open isomer

TABLE 4
Data for chromophores according to approach 1 for photostability

| | | | | | | | | | Chromophore degradation | |
|---|---|---|---|---|---|---|---|---|---|---|
| | | | | | | | | | relative to S340 (interpolated | |
| | | | | | | | | | to 0.5 MJ/m² of energy | |
| | | | | | | | | | exposure at 340 nm) | |
| | | | | | | | | | Sum of | |
| | | | | | | | | | degradation | |
| | | | | | | | | | product | |
| | | | | | | | | | percentages | |
| | | | Percentages of chromophore remaining and degradation products | | | | | | relative to | |
| | | Exposure | linearly interpolated to 0.5 MJ/m2 @ 340 nm of exposure energy | | | | | | the sum of | Ratio of |
| | | energy | | | | | | Other | degradation | chromophore |
| | Weathering | (MJ/m2 @ | Chromophore | | | | | degradation | product | remaining |
| Chromophore | time | 340 nm, | Remaining | Minus-HF | Minus-2HF | Ketone | Sulfone | products | percentages | relative to |
| (wt %) | (h) | 82° C. bp) | (%) | (%) | (%) | (%) | (%) | (%) | for S340 | S340 |
| S340 (3%) | 1533 | 3.75 | 91.1 | 3.5 | 1.0 | 0.0 | 0.0 | 4.4 | 1.00 | 1.00 |
| S343 (3%) | 970 | 2.37 | 85.2 | 0.0 | 3.1 | 0.0 | 0.0 | 11.8 | 1.67 | 0.93 |
| S344 (3%) | 349 | 0.85 | 65.1 | 25.7 | 8.0 | 0.0 | 0.0 | 1.2 | 3.93 | 0.71 |
| S345 (3%) | 970 | 2.37 | 86.9 | 6.9 | 1.9 | 0.0 | 0.0 | 4.4 | 1.48 | 0.95 |
| S362 (10%) | 157 | 0.38 | 55.5 | 37.6 | 4.8 | 2.1 | 0.0 | 0.0 | 5.01 | 0.61 |
| S364 (10%) | 550 | 1.35 | 80.4 | 14.9 | 2.7 | 0.6 | 0.0 | 1.4 | 2.21 | 0.88 |
| S367 (10%) | 207 | 0.51 | 40.8 | 56.0 | 0.0 | 1.1 | 0.0 | 2.1 | 6.67 | 0.45 |
| S374 (10%) | 299 | 0.73 | 72.4 | 26.5 | 0.0 | 0.0 | 0.0 | 1.1 | 3.11 | 0.79 |
| S378 (3%) | 314 | 0.77 | 92.5 | 0.0 | 0.0 | 0.0 | 7.5 | 0.0 | 0.84 | 1.02 |
| S383 (10%) | 453 | 1.11 | 100.0 | 0.0 | 0.0 | 0.0 | 0.0 | 0.0 | 0.00 | 1.10 |
| S384 (10%) | 365 | 0.89 | 97.2 | 0.0 | 0.0 | 0.0 | 1.3 | 1.5 | 0.32 | 1.07 |

Approach 2

Alternatively, the photostability of chromophores may be increased by strategic design of the substitution pattern of the terthiophene moiety. Chromophores with a substituent in the 4-position of the internal thiophene that have equal or greater steric size relative to the substituent in the 5-position exhibit high photostability regardless of the substitution pattern on the benzofuran moiety (see chromophores S193, S377, S381, S386, S387, S388, S390, S392, S396, S398, S400, S404 and S405 in Tables 5 and 6). Without wishing to be bound by any particular theory, it is postulated that the origin of this enhanced photostability is a result of altering the preferred conformation of the internal thiophene to favour the “head-to-tail” arrangement of the terthiophene where the origin of this conformational bias arises from steric interactions between substituents in the 4- and 5-position of the internal thiophene and the neighbouring benzofuran group. By installing alkyl or heteroalkyl groups, such as, for example, tert-butyl groups in the 4- and 4″-positions or using a ring structure to alkylate the 4,5- and 4″,5″-positions of the terthiophene (S377 and S381, respectively), the undesired cyclization reaction leading to the Minus-HF degradation product is disfavoured. In the case of ester (S386 and S387, respectively) and polyether (S392 and S388, respectively) chains on the benzofuran, the terthiophene substitution pattern prevents the Minus-HF product, counter to the behavior observed in the S374 and S364 counterparts, where the substituent in the 5-position of the internal thiophene is sterically larger than the substituent in the 4-position.

TABLE 5
Data for chromophores according to approaches 1 and 2 for
absorbance of ring-open isomer at 400 nm, colour, photostationary
state, solubility, and oxidation potential

| | | | | | Solubility | | |
|---|---|---|---|---|---|---|---|
| | | | | | in | | |
| | Abs @ | | | | Rhodia- | | |
| Chromo- | 400 | | λₘₐₓ | PSS | solv | RC | RO |
| phore | nm, RO¹ | YI² | (nm) | (SS) | IRIS | [O]³ | [O]⁴ |
| S193 | 0.097 | 3.59 | 587 | 0.269 | not | 1.02 | 1.41 |
| | | | | | avail- | | |
| | | | | | able | | |
| S340 | 0.128 | 5.61 | 591 | 0.417 | 7% | 0.95 | 1.35 |
| S343 | 0.11 | 4.87 | 573 | 0.315 | <1% | 1.1 | 1.37 |
| S344 | 0.1 | 2.91 | 583 | 0.337 | <3% | 1.05 | 1.36 |
| S345 | 0.0809 | 3.32 | 575 | 0.255 | <3% | 1 | 1.38 |
| S362 | 0.088 | 2.79 | 583 | 0.353 | >10% | 1.06 | 1.33 |
| S364 | 0.105 | 4.21 | 593 | 0.427 | >10% | 0.93 | 1.32 |
| S367 | 0.102 | 5.47 | 583 | 0.32 | >10% | 1.12 | 1.38 |
| S374 | 0.144 | 6.76 | 590 | 0.422 | >10% | 0.93 | 1.35 |
| S377 | 0.101 | 4.89 | 593 | 0.308 | >5% | 0.97 | 1.42 |
| S378 | 0.127 | 4.94 | 593 | 0.351 | <5% | 0.9 | 1.36 |
| S381 | 0.131 | 8.12 | 594 | 0.358 | <3% | 0.91 | 1.29 |
| S383 | 0.144 | 4.52 | 590 | 0.408 | >10% | 0.91 | 1.36 |
| S384 | 0.107 | 4.13 | 593 | 0.342 | >10% | 0.87 | 1.31 |
| S386 | 0.113 | 4.05 | 593 | 0.351 | >10% | 0.99 | 1.4 |
| S387 | 0.146 | 8.15 | 594 | 0.409 | >10% | 0.92 | 1.29 |
| S388 | 0.132 | 6.3 | 600 | 0.38 | >10% | 0.89 | 1.28 |
| S390 | 0.133 | 6.48 | 593 | 0.45 | >10% | 0.93 | 1.29 |
| S392 | 0.103 | 4.29 | 594 | 0.319 | >10% | 0.95 | 1.39 |
| S396 | 0.08 | 2.78 | 578 | 0.278 | >3% | 1.18 | 1.48 |
| S398 | 0.136 | 4.72 | 585 | 0.364 | >10% | 1.05 | 1.42 |
| S400 | 0.125 | 6.67 | 592 | 0.306 | >3% | 1.01 | 1.29 |
| S404 | 0.182 | 11.1 | 599 | 0.429 | n/a | n/a | n/a |
| S405 | 0.143 | 7.26 | 598 | 0.381 | >1% | 0.91 | 1.28 |

¹Absorption of ring-open isomer at 400 nm
²Yellowness Index
³oxidation potential of ring-closed isomer
⁴oxidation potential of ring-open isomer

TABLE 6
Data for chromophores according to approaches 1 and 2 for
photostability

| | | | | | | | | | Chromophore degradation | |
|---|---|---|---|---|---|---|---|---|---|---|
| | | | | | | | | | relative to S340 (interpolated | |
| | | | | | | | | | to 0.5 MJ/m² of energy | |
| | | | | | | | | | exposure at 340 nm) | |
| | | | | | | | | | Sum of | |
| | | | | | | | | | degradation | |
| | | | | | | | | | product | |
| | | | | | | | | | percentages | |
| | | | Percentages of chromophore remaining and degradation products | | | | | | relative to | |
| | | Exposure | linearly interpolated to 0.5 MJ/m2 @ 340 nm of exposure energy | | | | | | the sum of | Ratio of |
| | | energy | | | | | | Other | degradation | chromophore |
| | Weathering | (MJ/m2 @ | Chromophore | | | | | degradation | product | remaining |
| Chromophore | time | 340 nm, | Remaining | Minus-HF | Minus-2HF | Ketone | Sulfone | products | percentages | relative to |
| (wt %) | (h) | 82° C. bp) | (%) | (%) | (%) | (%) | (%) | (%) | for S34 | S340 |
| S193 | 408 | 1.00 | 92.7 | 1.4 | 2.1 | 0.0 | 0.0 | 3.8 | 0.82 | 1.02 |
| (3%) | | | | | | | | | | |
| S340 | 1533 | 3.75 | 91.1 | 3.5 | 1.0 | 0.0 | 0.0 | 4.4 | 1.00 | 1.00 |
| (3%) | | | | | | | | | | |
| S343 | 970 | 2.37 | 85.2 | 0.0 | 3.1 | 0.0 | 0.0 | 11.8 | 1.67 | 0.93 |
| (3%) | | | | | | | | | | |
| S344 | 349 | 0.85 | 65.1 | 25.7 | 8.0 | 0.0 | 0.0 | 1.2 | 3.93 | 0.71 |
| (3%) | | | | | | | | | | |
| S345 | 970 | 2.37 | 86.9 | 6.9 | 1.9 | 0.0 | 0.0 | 4.4 | 1.48 | 0.95 |
| (3%) | | | | | | | | | | |
| S362 | 157 | 0.38 | 55.5 | 37.6 | 4.8 | 2.1 | 0.0 | 0.0 | 5.01 | 0.61 |
| (10%) | | | | | | | | | | |
| S364 | 550 | 1.35 | 80.4 | 14.9 | 2.7 | 0.6 | 0.0 | 1.4 | 2.21 | 0.88 |
| (10%) | | | | | | | | | | |
| S367 | 207 | 0.51 | 40.8 | 56.0 | 0.0 | 1.1 | 0.0 | 2.1 | 6.67 | 0.45 |
| (10%) | | | | | | | | | | |
| S374 | 299 | 0.73 | 72.4 | 26.5 | 0.0 | 0.0 | 0.0 | 1.1 | 3.11 | 0.79 |
| (10%) | | | | | | | | | | |
| S377 | 491.5 | 1.20 | 100.0 | 0.0 | 0.0 | 0.0 | 0.0 | 0.0 | 0.00 | 1.10 |
| (5%) | | | | | | | | | | |
| S378 | 314 | 0.77 | 92.5 | 0.0 | 0.0 | 0.0 | 7.5 | 0.0 | 0.84 | 1.02 |
| (3%) | | | | | | | | | | |
| S381 | 348 | 0.85 | 92.3 | 1.3 | 0.0 | 3.3 | 3.1 | 0.0 | 0.87 | 1.01 |
| (1%) | | | | | | | | | | |
| S383 | 453 | 1.11 | 100.0 | 0.0 | 0.0 | 0.0 | 0.0 | 0.0 | 0.00 | 1.10 |
| (10%) | | | | | | | | | | |
| S384 | 365 | 0.89 | 97.2 | 0.0 | 0.0 | 0.0 | 1.3 | 1.5 | 0.32 | 1.07 |
| (10%) | | | | | | | | | | |
| S386 | 579.5 | 1.42 | 98.6 | 0.0 | 0.0 | 0.0 | 0.0 | 1.4 | 0.16 | 1.08 |
| (10%) | | | | | | | | | | |
| S387 | 527 | 1.29 | 100.0 | 0.0 | 0.0 | 0.0 | 0.0 | 0.0 | 0.00 | 1.10 |
| (10%) | | | | | | | | | | |
| S388 | 579.5 | 1.42 | 97.7 | 0.0 | 0.0 | 0.2 | 0.4 | 1.7 | 0.26 | 1.07 |
| (10%) | | | | | | | | | | |
| S390 | 579.5 | 1.42 | 98.8 | 0.0 | 0.0 | 0.0 | 0.0 | 1.2 | 0.14 | 1.08 |
| (10%) | | | | | | | | | | |
| S392 | 579 | 1.42 | 98.6 | 0.0 | 0.0 | 0.0 | 0.1 | 1.3 | 0.16 | 1.08 |
| (10%) | | | | | | | | | | |
| S396 | 408 | 1.00 | 97.9 | 0.0 | 0.0 | 1.0 | 0.0 | 1.1 | 0.24 | 1.07 |
| (3%) | | | | | | | | | | |
| S398 | 162 | 0.40 | 95.3 | 0.0 | 0.0 | 0.0 | 0.0 | 4.7 | 0.53 | 1.05 |
| (10%) | | | | | | | | | | |
| S400 | 476 | 1.17 | 96.7 | 0.0 | 1.4 | 0.8 | 0.0 | 1.1 | 0.37 | 1.06 |
| (3%) | | | | | | | | | | |
| S404 | 805 | 1.97 | 100.0 | 0.0 | 0.0 | 0.0 | 0.0 | 0.0 | 0.00 | 1.10 |
| (1%) | | | | | | | | | | |
| S405 | 701 | 1.72 | 97.1 | 0.0 | 0.0 | 2.9 | 0.0 | 0.0 | 0.33 | 1.07 |
| (1%) | | | | | | | | | | |

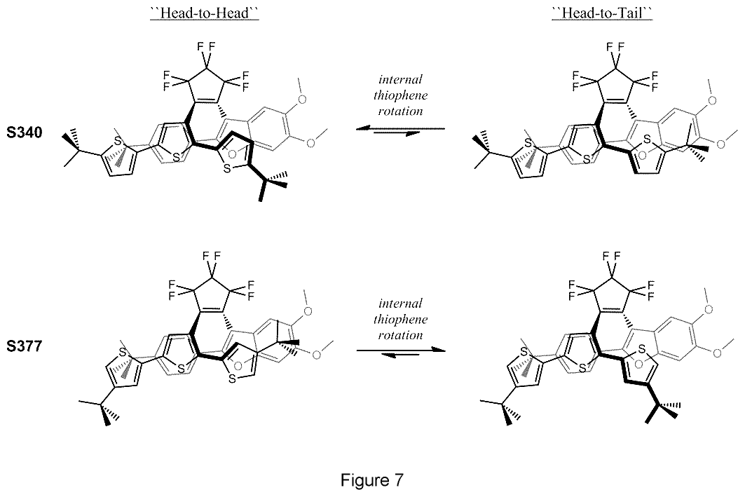

FIG. 7 shows a comparison between S340 and S377 with respect to internal thiophene rotation. Bold atoms in S340 illustrates the favourable orientation of the tert-butyl group so as to minimize steric interactions with the benzofuran being in the “head-to-head” orientation. This orientation brings the 4′-carbon of terthiophene into close proximity with the anchoring octafluorocyclopentene, which is the first step in formation of the Minus-HF degradation product. In contrast, S377, which has a substituent at R_{6b} that is of larger steric size than the substituent at R₆ₐ, favours a “head-to-tail” orientation which precludes the deleterious ring closure which leads to the formation of the Minus-HF degradation product.

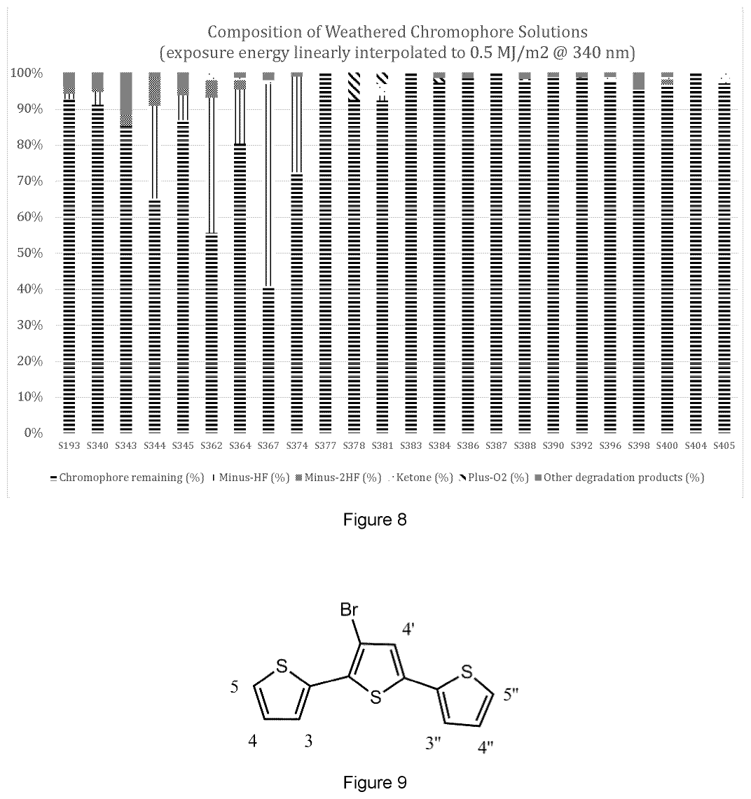

As shown in FIG. 8, in all cases chromophores developed under Approach 1 or 2 and dissolved in solvent at room temperature exhibited less than 10% chromophore degradation at 0.5 MJ/m2 of weathering, thus meeting the minimum performance criteria for test compounds for commercial product development. Furthermore, these compounds could also be structurally modified with solubilizing groups to have a solubility greater than 6 wt % in Rhodiasolv IRIS solvent. Thus, compared to other diarylethene compounds, the chromophores disclosed herein have superior properties that allow them to be incorporated into commercial optical filter or exterior glazing products.

Switching Materials

Some switching materials are described in PCT Publications WO2010/142019 and WO2013/152425. In addition to the chromophore and a cathodic species, a switching material may include one or more of a crosslinkable polymer, a polymer, a salt, a cross-linker, a hardener, an accelerant (catalyst), or a co-solvent. A switching material has both electrochromic and photochromic properties. A switching material may darken (e.g. reach a ‘dark state’) when exposed to ultraviolet (UV) light from a light source, and may lighten (“fade”, achieve a ‘light state”) when exposed to an electric charge. In some embodiments, the switching material may fade upon exposure to selected wavelengths of visible (VIS) light (“photofade”, “photobleach”), without sacrifice of the ability to be electrofaded when restored to a darkened state. In some embodiments, the switching material may darken when exposed to light comprising wavelengths from about 350 nm to about 475 nm, or any amount or range therebetween, and may lighten when a voltage is applied, or when exposed to light comprising wavelengths from about 500 to about 700 nm. The switching material may be optically clear. The switching material may be a thermoplastic, thermosetting (uncured) or thermoset (cured) material. The switching material may be a viscoelastic material. The switching material may be cured by heating, exposure to UV light, chemical reaction, irradiation, electron beam processing or a combination thereof.

In various embodiments, the switching material, or an optical filter comprising the switching material, may be disposed upon a transparent conductive electrode such as a pane of glass that has a conductive coating applied to it, a transparent conductive polyester film, or other transparent conductive material suitable for use as a window. In various embodiments, the switching material may have bus bars attached to the electrodes to allow connection to a power supply. In various embodiments, the switching material may be edge sealed. In various embodiments, the switching material may be incorporated into an insulated glazing unit (IGU), or a storm window or secondary glazing. Methods of making an IGU, windows or the like, and affixing an optical filter to glass or other suitable material are described in, for example, WO2010/142019 as are methods of configuring an electrical system and/or control system for operation (electrofading) of an IGU. In some embodiments, the switching material may be incorporated into an ophthalmic device (e.g. visors, masks, goggles, lenses, eyeglasses or the like). In some embodiments, the switching material may be used in glazing products such as architectural installations or vehicle (e.g. truck, car, airplane, train or the like) installations. Architectural installations may be external-facing, or internal to the building, and may include a window, a wall or a display. Vehicle installations include windows, sunroofs or other glazings, including sunroofs of various types including pop-up, spoiler, inbuilt, folding sunroofs, panoramic roof systems or removable roof panels. Vehicle windows include windshields, rear windows, side windows, sidelight windows, internal dividers to divide the interior space of a vehicle for temporary or permanent purposes. Electrical power may be provided by a separate battery, or the device may be connected to an electrical system of the device, for example, it may be wired into a vehicle or building's electrical system.

### EXAMPLES

These examples illustrate various aspects of the invention, evidencing a variety of conditions for preparing mixed arm diarylethene compounds. Selected examples are illustrative of advantages that may be obtained compared to alternative methods, and these advantages are accordingly illustrative of particular embodiments and not necessarily indicative of the characteristics of all aspects of the invention.

As used herein, the term “about” refers to an approximately +/−10% variation from a given value. It is to be understood that such a variation is always included in any given value provided herein, whether or not it is specifically referred to.

All solvents were dried prior to use or purchased as anhydrous. Where necessary, solvents were degassed by bubbling with argon or nitrogen. Solvents for NMR analysis (Cambridge Isotope Laboratories) were used as received.

Column chromatography was performed using silica gel 60 (230-400 mesh) from Silicycle Inc. Octafluorocyclopentene was purchased from SynQuest and catalysts Pd(dppf)Cl₂ and Pd(PPh₃)₄ were purchased from Strem. All other synthetic precursors, solvents and reagents were purchased from Aldrich, Anachemia or Caledon. ¹H NMR characterizations were performed on a Bruker AMX 400 instrument working at 400.103 MHz. ¹³C NMR characterizations were performed on a Bruker AMX 400 instrument working at 100.610 MHz. Chemical shifts (δ) are reported in parts per million relative to tetramethylsilane (TMS) using the residual solvent peak as a reference standard. Coupling constants (J) are reported in Hertz. The ring-opening reactions were carried out using the light of a 150 W tungsten source that was passed through a 490 nm or a 434 nm cutoff filter to eliminate higher energy light.

Photostationary State (PSS)

UV/Vis spectra were obtained using an OceanOptics™ Spectrophotometer. A 2×10⁻⁵ M solution of compound in solvent was prepared, and photofaded using visible light until absorption in the visible region of the spectrum stabilized. The sample was then irradiated with simulated sunlight (QSUN SS-150 Solar Simulator with xenon arc lamp) until the absorption spectrum stabilized. To obtain PSS in the presence of a UV blocking film, a second sample was prepared and irradiated as described, with a UV blocking film inserted in the light path when irradiating.

Preparation of Chromophore Solutions

Chromophore solutions in Rhodiasolv IRIS were prepared at 1 to 10 wt % loading. Solutions were prepared in a glovebox by charging the chromophore and Rhodiasolv IRIS to a trace-clean vial, and the solution was stirred on a hotplate at 90° C. for 2-24 hours. After cooling, the chromophore solution was injected into glass weathering cells and the fill and vent ports were sealed with a thin disc of Kalrez (FFKM Perfluorinated Elastomer) and secured with a clamp and set-screw. The weathering cells were filled and sealed inside the glove box.

Measurement of Photostability

A. Fabrication of cuvettes. Two holes of 0.8 mm diameter were drilled at opposite ends in a 75×50 mm piece of float glass using a diamond drill bit in a rotary tool. The drilled glass and a matching piece of undrilled glass were then cleaned using hot soapy water, rinsed with deionized water and acetone and then dried with compressed air. A 5 mm wide frame was cut from a sheet of 50 μm thickness EVAL EF-F using a laser engraver. The EVAL frame was placed around the perimeter of one sheet of glass and sandwiched by the second sheet of glass. The glass-EVAL-glass sandwich was secured with tape and heated under vacuum (10 mmHg) for 30 minutes at 170° C. in a vacuum bag. After cooling, a third piece of float glass was PVB laminated to the non-drilled side of the sealed glass cells using Trosifol UV Extra Protect B150 PVB (400 nm UV cut-off).

B. Xenon arc weathering protocol. Devices were exposed to simulated weathering conditions inside a Xe-3-HS xenon arc chamber, manufactured by Q-labs Inc. The samples were weathered according to the following table. The devices were exposed to these conditions continuously, with no dark or spray cycles included.

| Parameter | Condition |
|---|---|
| Irradiance | 0.68 W/m² at 340 nm |
| Lamp Filter | Daylight Q |
| Temperature | 82° C. BPT |
| Relative humidity | Ambient (not Controlled) |

C. Analysis of weathered chromophore solutions by LCMS. The set-screws, metal clamps and Kalrez plugs were removed from the cuvette to expose the fill port. A 5 mL plastic syringe (NORM-JECT®) equipped with a tip (Precision Tips, 0.010″ RED) was inserted into one fill port and the chromophore solution was withdrawn from the cuvette. The chromophore solution was then injected into a 2 mL amber vial (part number 5182-0716, Agilent Technology) prefilled with 1.5 mL LC-MS grade acetonitrile (HiPerSolbe CHROMANORM Acetonitrile, VWR BDH Chemicals). An aliquot of the acetonitrile solution (2 μL) was injected into the LC-MS system (Agilent Infinity 1290 LC-MS system with diode array detector) through an autosampler mechanism. Species in the injected sample were separated through a reverse phase column (Zorbax SB-C18, 2.1×50 mm, 1.8 μm, operated at 40° C.), using a mixture of acetonitrile (mixed with 1% formic acid v/v ratio), methanol (HiPerSolbe CHROMANORM Methanol, VWR BDH Chemicals) and water (HiPerSolbe CHROMANORM Water, VWR BDH Chemicals) as mobile phase. Photostability performance is tabulated relative to a control sample that does not contain the indicated compound number.

Suitable chromophores for commercial applications should exhibit less than 10% degradation at 0.5 MJ/m² of simulated sunlight exposure measured at 340 nm and a black panel temperature of 82° C. in the accelerated chromophore solution screening test.

### Example 1: Selective Tert-Butylation of Terthiophene

Substitution of the 5 and 5″ positions of 3′-bromo-2,2′:5′2″-terthiophene, or another terthiophene substrate, can be done through Friedel-Crafts alkylation. FIG. 9 shows 3′-bromo-2,2′:5′,2″-terthiophene substituent positions labelled with IUPAC numbering. However, Friedel-Crafts alkylation also results in the formation of considerable quantities of constitutional isomers, based on LC-MS of the reaction product. Loss of selectivity to give 5,4″ and 4,5″ reaction products was observed, however, as conditions for the reaction were optimization, the formation of a 4,4″-substituted product became the major product of the reaction (equation (1)).

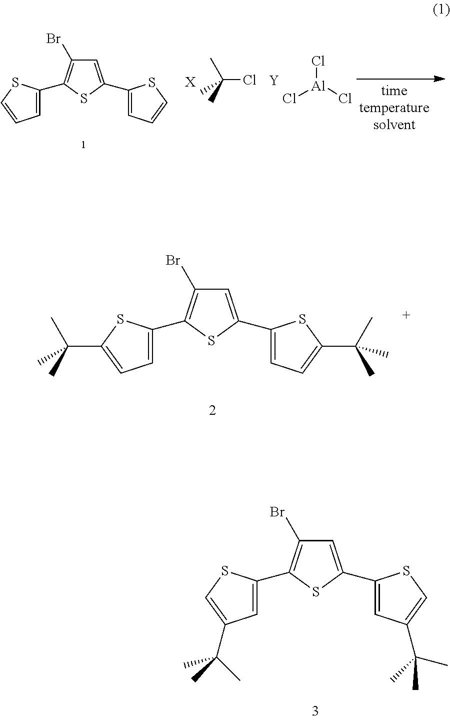

By varying the reaction temperature, tert-butyl chloride concentration, and Lewis acid concentration, the selectivity of the reaction could be changed. As shown in Table 7, subjecting 1 to excess Lewis acid and tert-butyl chloride is critical to achieving the 4,4″ selectivity. Entries 1, 2 and 5 show considerable excess of aluminum chloride and all generate 3 as the major product. Conversely, entry 4 shows that limited aluminum chloride, even with a large excess of tert-butyl chloride, generates 2 predominantly. However, the large quantities of Lewis acid also serve to generate deleterious biproducts. In particular “M+14” (meaning the parent mass plus 14 amu) correlates with 4 or more aluminum chloride equivalents (entries 1, 2 and 5).

TABLE 7
Reaction condition screening for 4,4″ tert-butylation optimization. All
reactions were conducted in dichloromethane (0.07M) at room temperature and proceeded
for 0.5 hours before analysis (HPLC-LCMS). Values are given as an “area %” of compounds
detected by 254 nm absorbance.

| | ^{t}BuCl | AlCl₃ | | | 5 or 5″- | | | di-tert- | Other |
|---|---|---|---|---|---|---|---|---|---|
| | equiv. | equiv. | | | monotert- | | M + 14 | butylated | alkylated |
| Entry | “X” | “Y” | 2 | 3 | butylated | 1 | biproduct | isomers | species |
| 1 | 5 | 5 | 0 | 56.0 | 0 | 0 | 20.8 | 0 | 18.6 |
| 2 | 4 | 4 | 2.2 | 57.7 | 1.3 | 0 | 18.4 | 1.7 | 7.2 |
| 3 | 4 | 3 | 14.1 | 43.8 | 13.0 | 0.6 | 0.9 | 7.3 | 9.1 |
| 4 | 4 | 2 | 72.8 | 1.8 | 3.4 | 0.5 | 6.7 | 6.3 | 2.2 |
| 5 | 3 | 4 | 1.75 | 56.5 | 3.4 | 0 | 14.7 | 4.3 | 7.9 |
| 6 | 3 | 3 | 4.6 | 37.0 | 7.0 | 0 | 5.6 | 4.9 | 3.9 |
| 7 | 2 | 2 | 49.4 | 8.3 | 21.0 | 0 | 4.3 | 11.1 | 0.5 |
| 8 | 2 | 1 | 8.8 | 0 | 59.8 | 29.9 | 0 | 0 | 0 |

Upon determining the temperature and reagent equivalents required to achieve 3 as the major product, yield was then optimized by eliminating the formation of by-products and minimizing purification.

Without wishing to be bound by theory, it appeared that the “M+14” by-product involved dichloromethane acting as an electrophile and other solvents of similar dielectric constant were evaluated. When performing the reaction in carbon tetrachloride (entry 6), very little reaction progress was observed after 0.5 h. Chloroform (entry 5) generated a similar result to dichloromethane (entry 4) but favored 2 and had similar biproduct profile. Electron-poor aromatics were ideal candidates. Nitrobenzene (entry 3) afforded a slow reaction to exclusively 2 with significantly improved biproduct profile. Finally, chlorobenzene and fluorobenzene (entries 1 and 2) showed exclusively 3 with minimal quantities of biproducts. The elevated boiling points of these solvents offered opportunity to increase the reaction concentration without technical concerns of the exotherm.

TABLE 8
Solvent screening. All reactions were performed with 4 equivalents of
tert-butyl chloride and 3 equivalents of aluminum chloride and proceeded for 0.5 hours
before analysis (HPLC-LCMS). Values are given as an “area %” of compounds detected by
254 nm absorbance.

| | | | | 5 or 5″- | | | di-tert- | Other |
|---|---|---|---|---|---|---|---|---|
| | | | | monotert- | | M + 14 | butylated | alkylated |
| Entry | Solvent | 2 | 3 | butylated | 1 | biproduct | isomers | species |
| 1 | Chlorobenzene | 0.4 | 79.2 | 5.3 | 0 | 0 | 0.6 | 0.3 |
| 2 | Fluorobenzene | 0 | 96.0 | 2.5 | 0 | 0 | 0.2 | 1.2 |
| 3 | Nitrobenzene | 21.7 | 0 | 0.4 | 76.0 | 0 | 1.4 | 1.4 |
| 4 | Dichloromethane | 14.1 | 43.8 | 13.0 | 0.6 | 0.9 | 7.3 | 9.1 |
| 5 | Chloroform | 41.4 | 25.3 | 2.7 | 3.6 | 6.6 | 6.6 | 18.6 |
| 6 | Carbon tetrachloride | 1.0 | 0 | 0.7 | 97.3 | 0 | 0 | 0 |

Elevated temperature and large excess of alkylating reagents appears to flip the selectivity observed at lower temperature. As shown in equation 2, the optimal conditions for synthesizing the 5,5″ di-tert-butylated product 2 from 1 require 4 equivalents of tert-butyl chloride and 3 equivalents of aluminum chloride in dichloromethane at −78° C. The reaction mixture was quenched at −78° C. with water after 5 h. Inverse addition is also required in this case to minimize the production of constitutional isomers. Specifically, a precooled solution (−78° C.) of 1 in dichloromethane is added dropwise to a −78° C. dichloromethane solution of tert-butyl chloride and aluminum chloride.

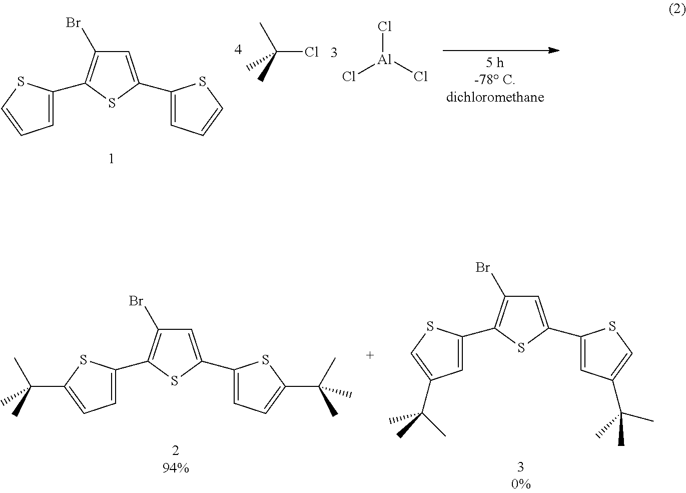

Conversely, as shown in equation 3, the optimal conditions for synthesizing the 4,4″ ditert-butylated product 3 from 1 require 5 equivalents of tert-butyl chloride and 5 equivalents of aluminum chloride in chlorobenzene at room temperature. The reaction mixture must be quenched with water after 0.5 h.

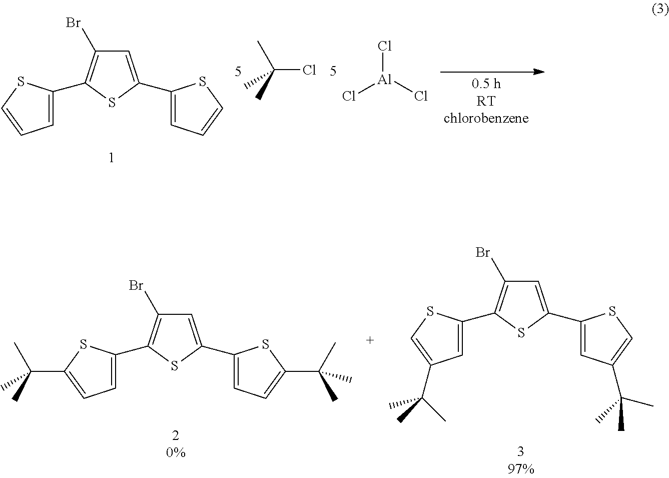

In various embodiments, the method of synthesizing a 4,4″-substituted 2,2′:5′,2″-terthiophene comprises reacting a terthiophene substrate with more than 2 molar equivalents of a Lewis acid and more than 2 molar equivalents of an electrophile at room temperature, wherein the terthiophene substrate is a polythiophene comprising more than two thiophenes, and wherein the 4,4″-substituted 2,2′:5′,2″-terthiophene is synthesized in excess of a 5,5″-substituted 2,2′:5′,2″-terthiophene.

In various embodiments, the terthiophene substrate is 3′-bromo-2,2′:5′2″-terthiophene. In various embodiments the terthiophene substrate is 2,2′:5′2″-terthiophene.

In various embodiments, the Lewis acid is iron chloride, aluminum chloride, iron bromide, aluminum bromide or aluminum iodide. In various embodiments, the Lewis acid is aluminum chloride.

In various embodiments, the electrophile is an alkyl having a tertiary halide or an acylation reagent. In various embodiments, the electrophile is 2-chloro-2-methylpropane, 2-bromo-2-methylpropane, acetyl chloride or benzoyl chloride. In various embodiments, the electrophile is 2-chloro-2-methylpropane or 2-bromo-2-methylpropane.

In various embodiments, a yield of the 4,4″-substituted 2,2′:5′2″-terthiophene is 95% or greater.

In various embodiments, the terthiophene substituent is reacted with the Lewis acid and the electrophile in a solvent. In various embodiments, the solvent is a halogenated solvent with a boiling point greater than about 39° C. In various embodiments, the solvent is chlorobenzene, fluorobenzene, dichloromethane, chloroform, 1,2-dichloroethane or a combination thereof. In various embodiments, the solvent is chlorobenzene, fluorobenzene or a combination thereof.

### Example 2: Synthesis of 3′-bromo-5,5″-di-tert-butyl-2,2′:5′,2″-terthiophene

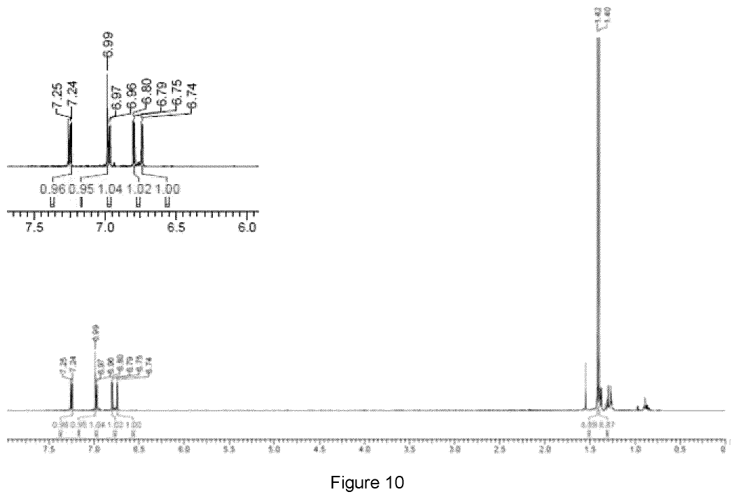

To a stirred solution of 2-chloro-2-methylpropane (2.99 ml, 27.5 mmol) in dry dichloromethane (90 ml) at −78° C. was added aluminum trichloride (2.81 g, 21.08 mmol) and the mixture was stirred for 1 h. A solution of 3′-bromo-2,2′:5′,2″-terthiophene (3.00 g, 9.17 mmol) in dichloromethane (60 ml) was cooled to −78° C. in a cooling jacketed dropping funnel and was then added over 0.5 h. An immediate colour change from pale yellow to deep purple was observed upon addition. The reaction mixture was stirred at −78° C. for 5 h, and an aliquot was taken (analyzed by LCMS) to confirm completeness. Aqueous hydrochloric acid (20 mL, 1 M) was added to quench the reaction and the mixture was warmed to RT. The layers were separated, the aqueous layer was extracted with dichloromethane (2×50 mL) and the combined organic extracts were dried over magnesium sulfate, filtered through a short pad of silica (˜50 g), flushed through with dichloromethane and concentrated to yield 3′-bromo-5,5″-di-tert-butyl-2,2′:5′,2″-terthiophene (2) as a pale yellow oil in 94% yield (3.77 g, 8.58 mmol). ¹H NMR (400 MHz, CDCl₃): δ 7.25 (d, J=3.9 Hz, 1H), 6.99 (s, 1H), 6.97 (d, J=3.7 Hz, 1H), 6.80 (d, J=3.7 Hz, 1H), 6.75 (d, J=3.7 Hz, 1H), 1.42 (s, 9H), 1.40 (s, 9H). The ¹H NMR spectrum of 2 in CDCl₃ is shown in FIG. 10.

### Example 3: Synthesis of 3′-bromo-4,4″-di-tert-butyl-2,2′:5′,2″-terthiophene

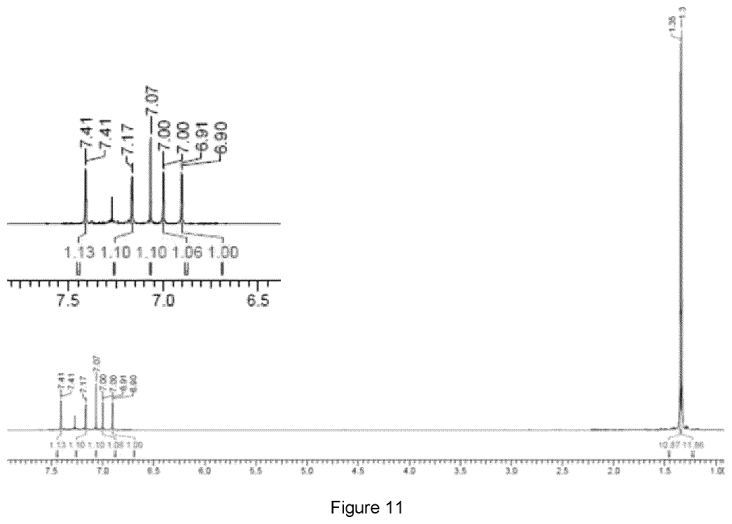

To a stirred solution of 3′-bromo-2,2′:5′,2″-terthiophene (7.50 g, 0.023 mol) and 2-chloro-2-methylpropane (12.46 mL, 0.115 mol) in chlorobenzene (100 mL, 0.22 M) was added aluminum chloride (15.28 g, 0.115 mol) over 2 minutes. The reaction mixture was monitored by LC-MS and upon completion after 0.5 h, 1 M aqueous hydrochloride acid was added. The phases were separated and the aqueous phase was extracted with dichloromethane (3×50 mL). The combined organic extracts were dried over magnesium sulfate, filtered and concentrated in vacuo. The residue was dissolved in hexanes and vacuum filtered through a plug of silica gel. The eluted material 3′-bromo-4,4″-di-tert-butyl-2,2′:5′,2″-terthiophene (3) was concentrated in vacuo and isolated as a yellow/brown oil in 97% yield (9.8 g, 0.022 mol). ¹H NMR (400 MHz, CDCl₃): δ 7.41 (d, J=1.6 Hz, 1H), 7.17 (d, J=1.6 Hz, 1H), 7.07 (s, 1H), 7.00 (d, J=1.6 Hz, 1H), 6.91 (d, J=1.6 Hz, 1H), 1.35 (s, 9H), 1.31 (s, 9H). The H NMR spectrum of 3 in CDCl₃ is shown in FIG. 11.

### Synthesis of Chromophore Arms

### Example 4: Preparation of 378 Arm

To a flask charged with magnesium turnings (1.534 g, 63.1 mmol), 2-bromo-3-methylthiophene (10.25 g, 57.9 mmol) in diethyl ether (70 ml) was added dropwise over 0.5 h. The reaction mixture was heated to reflux for 2 h. After cooling to room temperature, the solution was added dropwise to a stirred solution of 4,5-dibromo-5′-(tert-butyl)-2,2′-bithiophene (20 g, 52.6 mmol) and Pd(dppf)Cl₂ (0.192 g, 0.263 mmol) (dppf is 1,1′-Bis(diphenylphosphino)ferrocene) in tert-butyl methyl ether (210 ml) at room temperature. The reaction was stirred at room temperature 16 h. The solution was quenched by slow addition of methanol (15 ml) and poured over ice. Aqueous 10% hydrochloric acid was added (10 mL), the phases were separated, and the aqueous phase was extracted with dichloromethane. The combined organic extracts were washed with brine (200 mL), dried over magnesium sulfate and concentrated under reduced pressure. The product, 3′-bromo-5″-(tert-butyl)-3-methyl-2,2′:5′,2″-terthiophene (10.9 g, 27.4 mmol, 52% yield) was purified by column chromatography (hexanes eluent) and precipitated from methanol.

To a stirred solution of 3′-bromo-5″-(tert-butyl)-3-methyl-2,2′:5′,2″-terthiophene (10.9 g, 27.4 mmol) and 2-chloro-2-methylpropane (3.28 ml, 30.2 mmol) in dry dichloromethane (300 ml) at −78° C., aluminum trichloride (4.86 g, 36.5 mmol) was added. The reaction mixture was stirred at this temperature for 4 h, quenched with aqueous hydrochloric acid (10 mL) and warmed to room temperature. The mixture was poured into ice water and the biphasic mixture was separated, the aqueous portion was extracted with dichloromethane (2*150 mL). The combined organic extracts were dried over magnesium sulfate and filtered through a short silica pad (˜50 g), flushed with dichloromethane. Upon concentration under reduced pressure, the product 3′-bromo-5,5″-di-tert-butyl-3-methyl-2,2′:5′,2″-terthiophene (11.6 g, 25.6 mmol, 93% yield) was isolated without further purification.

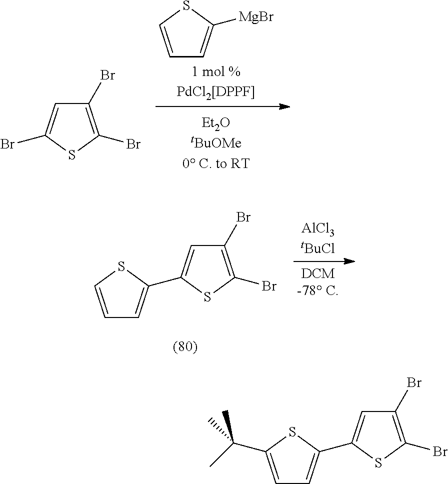

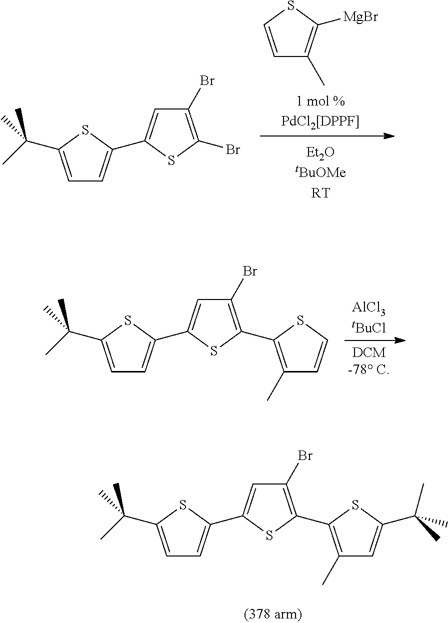

### Example 5: Preparation of 377 Arm

To a solution of 3′-bromo-2,2′:5′,2″-terthiophene (15.0 g, 0.046 mol) and 2-chloro-2-methylpropane (21.21 g, 0.229 mol) in chlorobenzene (200 mL) at room temperature, aluminum trichloride (30.6 g, 0.229 mol) was added over two minutes. The solution was stirred for 0.5 h and quenched with aqueous hydrochloric acid (100 mL). the phases were separated and the organic phase was concentrated under reduced pressure. The product, 3′-bromo-4,4″-di-tert-butyl-2,2′:5′,2″-terthiophene (19.14 g, 0.044 mol, 95% yield) was purified by column chromatography (hexanes eluent) and isolated as an orange oil.

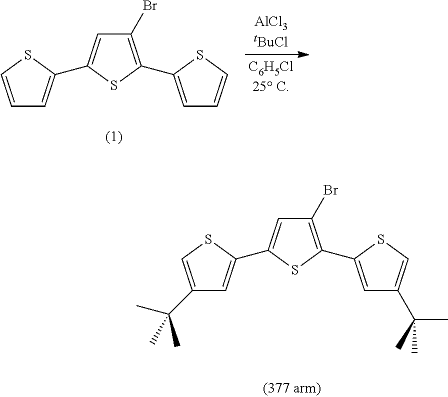

### Example 6: Preparation of 379 Arm

To a flask charged with magnesium turnings (3.0 g, 123 mmol), 2-bromo-3-methylthiophene (13.57 ml, 120 mmol) in diethyl ether (75 ml) was added dropwise over 0.75 h. The reaction mixture was heated to reflux for 2 h. After cooling to room temperature, the solution was added dropwise to a stirred solution of 2,3,5-tribromothiophene (7.59 ml, 58.8 mmol) and Pd(dppf)Cl₂ (0.108 g, 0.147 mmol) in tert-butyl methyl ether (188 ml) at 0° C. The reaction was allowed to warm to room temperature and stirred for 16 h. The solution was quenched by slow addition of methanol (15 ml) and poured over ice. Aqueous 10% hydrochloric acid was added (10 mL), the phases were separated, and the aqueous phase was extracted with diethylether. The combined organic extracts were washed with brine (200 mL), dried over magnesium sulfate and concentrated under reduced pressure. The product, 3′-bromo-3,3″-dimethyl-2,2′:5′,2″-terthiophene (17.6504 g, 49.7 mmol, 85% yield) was purified by column chromatography (hexanes eluent) and precipitated from methanol.

To a stirred solution of 3′-bromo-3,3″-dimethyl-2,2′:5′,2″-terthiophene (17.6504 g, 49.7 mmol) and 2-chloro-2-methylpropane (16.21 ml, 149 mmol) in dry dichloromethane (480 ml) at −78° C., aluminum trichloride (15.23 g, 114 mmol) was added. The reaction mixture was stirred at this temperature for 4 h, quenched with aqueous hydrochloric acid (10 mL) and warmed to room temperature. The mixture was poured into ice water and the biphasic mixture was separated, the aqueous portion was extracted with dichloromethane (2*150 mL). The combined organic extracts were dried over magnesium sulfate and filtered through a short silica pad (˜50 g), flushed with dichloromethane. Upon concentration under reduced pressure, the product 3′-bromo-5,5″-di-tert-butyl-3,3″-dimethyl-2,2′:5′,2″-terthiophene (19.1 g, 40.9 mmol, 82% yield) was isolated without further purification.

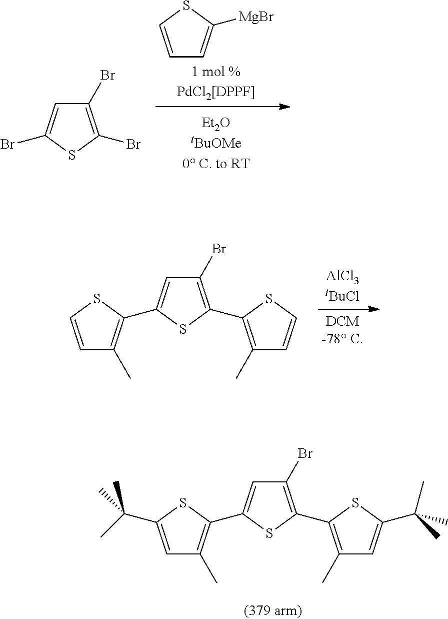

### Example 7: Preparation of 381 Arm

To a solution of 3′-bromo-2,2′:5′,2″-terthiophene (60.0 g, 0.183 mol) and 2,5-dichloro-2,5-dimethylhexane (67.15 g, 0.37 mol) in dichloromethane (1.50 L) cooled to −78° C., aluminum trichloride (73.3 g, 0.55 mol) was added. The reaction mixture was stirred at −78° C. for 2 h. Additional 2,5-dichloro-2,5-dimethylhexane (16.8 g, 0.09 mol) and aluminum trichloride (48.9 g, 0.37 mol) were added. The solution was warmed to −20° C. and quenched with aqueous hydrochloric acid (100 mL). The phases were separated and the organic phase was concentrated under reduced pressure. The product, 2,2′-(3-bromothiophene-2,5-diyl)bis(4,4,7,7-tetramethyl-4,5,6,7-tetrahydrobenzo[b]thiophene) (86.0 g, 0.16 mol, 85.7% yield) was purified by column chromatography (hexanes eluent) and precipitated from methanol.

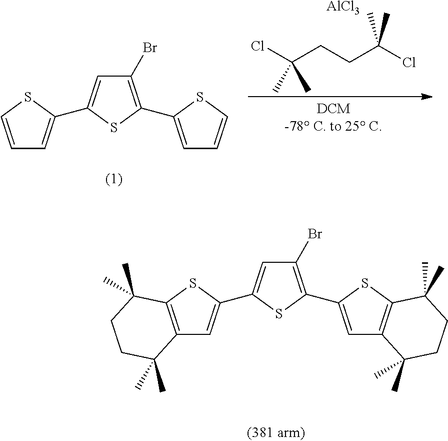

### Example 8: Preparation of 404 Arm

A mixture containing 2,4-dimethyl-2,4-pentanediol (12.000 g, 0.091 mol) and concentrated hydrochloric acid solution (12M, 78.2 mL, 0.908 mol) was stirred at room temperature for 30 min and then quenched with water (150 mL). The resultant mixture was extracted with diethyl ether (3×50 mL). The organic extracts were combined, dried over sodium sulfate and concentrated in vacuo. The crude material was purified by flash chromatography using hexanes as eluent to afford the desired product, 2,4-dichloro-2,4-dimethylpentane (2.760 g, 0.016 mol, 18.0%) as a colourless volatile oil.

To a stirred solution of 4-bromo-5-(thiophen-2-yl)-2,2′-bithiophene (1.32 g, 4.03 mmol) and 2,4-dichloro-2,4-dimethylpentane (1.5 g, 8.87 mmol) in anhydrous dichloromethane (24 mL) at −78° C. was added aluminum trichloride (2.15 g, 16.1 mmol). The reaction solution was stirred at −78° C. for 1 hour then slowly warmed to −20° C. Upon reaching −20° C., the reaction was quenched with water (5 mL). The phases were separated and the aqueous phase was extracted with dichloromethane (3×50 mL). The combined organic extracts were dried over sodium sulfate and concentrated in vacuo. The crude material was purified by flash chromatography using hexanes as eluent to afford the desired product, 3-bromo-2,5-bis({4,4,6,6-tetramethyl-4H,5H,6H-cyclopenta[b]thiophen-2-yl})thiophene, (0.288 g, 0.55 mmol, 13.7%) as a yellow oil.

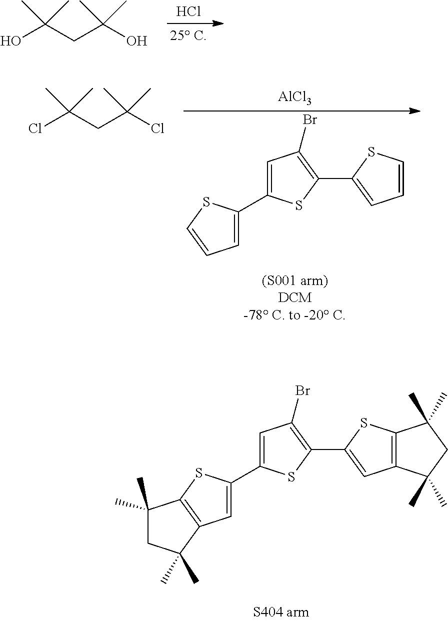

### Example 9: Preparation of 405 Arm

A stirred solution of diethyl glutarate (10.000 g, 0.062 mol) in anhydrous THF (100.0 mL) was added dropwise to a solution of methyl magnesium chloride in THF (3.0M, 83.2 mL, 0.250 mol) at −78° C. The cooling bath was removed and the resulting solution was stirred at room temperature for 1 hour. The reaction was carefully quenched with water at 0° C. The resultant mixture was extracted with Et₂O (3×50 mL), dried over MgSO₄ and concentrated in vacuo. The crude material was purified by flash chromatography using hexanes:ethyl acetate (3:1) as eluent to afforded the product 2,6-dimethyl-2,6-hepta-diol (4.7 g, 0.029 mmol, 47% yield) as a colourless oil.

A mixture containing 2,6-dimethyl-2,6-heptanediol (4.7 g, 0.029 mmol) and concentrated aqueous HCl (42.9 mL, 0.497 mol) was stirred at room temperature for 16 h. Water (300 mL) was added and the solid was collected by filtration. The filter cake was dissolved in diethyl ether (600 mL), washed with water (3×300 mL) and concentrated in vacuo. The crude crystalline solid was then charged with water (200 mL) and melted at 80° C. The bi-phasic solution was allowed to cool to room temperature for recrystallization to afford 2,6-dichloro-2,6-dimethylheptane (4.63 g, 0.023 mol, 80.1% yield) as a white crystalline solid.

To a stirred solution of 4-bromo-5-(thiophen-2-yl)-2,2′-bithiophene (2.000 g, 0.006 mol) and 2,6-dichloro-2,6-dimethylheptane (2.53 g, 0.013 mol) in anhydrous dichloromethane (37.0 mL) at −78° C., aluminum trichloride (0.367 g, 0.003 mol, 3.000 eq) was added over 10 mins. The reaction mixture was stirred and allowed to warm up over 3 h to −20° C. before being quenched by water (10 mL). The aqueous layer was removed and the organic layer was dried over sodium sulfate and concentrated in vacuo. The crude mixture was purified by flash chromatography using hexanes as eluent, followed by trituration in methanol (150 mL) to afford 3-bromo-2,5-bis({4,4,8,8-tetramethyl-4H,5H,6H,7H,8H-cyclohepta[b]thiophen-2-yl})thiophene (2.42 g, 0.004 mol, 68.8% yield) as a yellow powder.

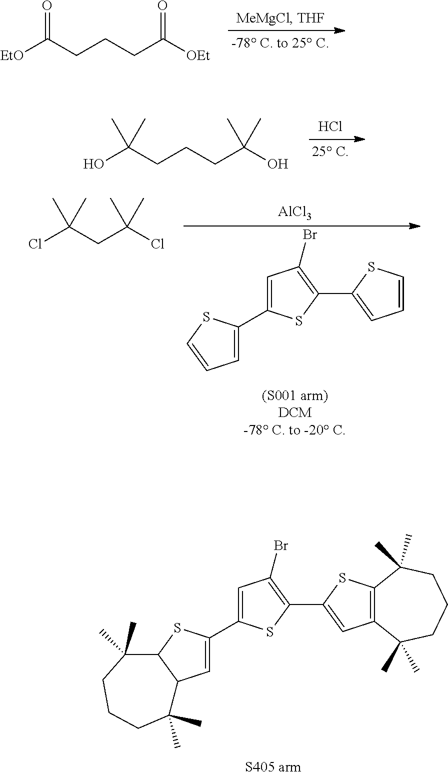

### Synthesis of Chromophores

### Example 10: Preparation of Chromophore S193

A stirred solution of 3′-bromo-2,2′:5′,2″-terthiophene (10 g, 30.6 mmol) in anhydrous diethyl ether (100 mL) was cooled to −78° C. n-butyllithium (12.22 ml, 2.5 M in hexanes, 30.6 mmol) was added dropwise over 30 min. The mixture was stirred at −78° C. for 15 mins and a solution of 3-(2-([2,2′:5′,2″-terthiophen]-3′-yl)-3,3,4,4,5,5-hexafluorocyclopent-1-en-1-yl)-2-(4-(tert-butyl)phenyl)-5,6-dimethoxybenzofuran (10.23 g, 20.37 mmol) in diethyl ether (15 mL) was added to the reaction mixture in one portion, the cold bath was removed and the mixture was warmed to room temperature and stirred for 2 h. Methanol (20 mL) was added to quench followed by concentration under reduced pressure. The product, 3-(2-([2,2′:5′,2″-terthiophen]-3′-yl)-3,3,4,4,5,5-hexafluorocyclopent-1-en-1-yl)-2-(4-(tert-butyl)phenyl)-5,6-dimethoxybenzofuran (7.04 g, 9.63 mmol, 47.3% yield), was obtained by column purification (hexanes/ethyl acetate eluent) and recrystallized from boiling methanol.

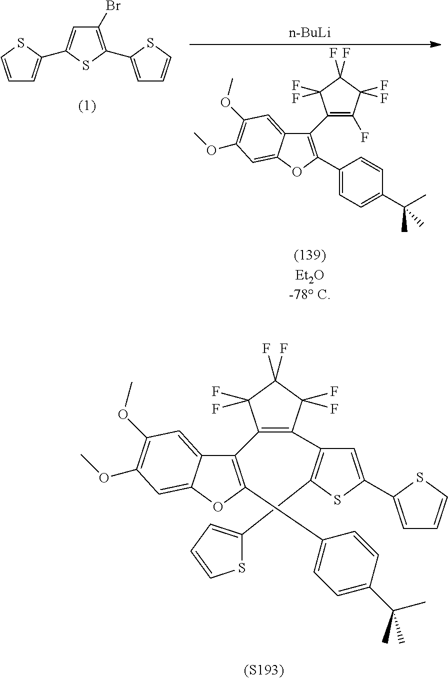

### Example 11: Preparation of Chromophore S340

A stirred solution of 3′-bromo-5,5″-di-tert-butyl-2,2′:5′,2″-terthiophene (5 g, 11.38 mmol) in anhydrous THF (32 mL) and DME (21 mL) was cooled to −78° C. n-butyllithium (4.55 ml, 2.5 M in hexanes, 11.38 mmol) was added dropwise over 30 min. The mixture was stirred at −78° C. for 15 mins and a solution of 2-(4-(tert-butyl)phenyl)-5,6-dimethoxy-3-(perfluorocyclopent-1-en-1-yl)benzofuran (4.76 g, 9.48 mmol) in THF (10 mL) was added to the reaction mixture in one portion, the cold bath was removed and the mixture was warmed to room temperature and stirred for 2 h. Methanol (20 mL) was added to quench followed by concentration under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)-5,6-dimethoxybenzofuran (4 g, 4.74 mmol, 50.0% yield) was obtained by column purification and precipitation from methanol. ¹H NMR (400 MHz, CDCl₃) δ 7.23-7.18 (m, 4H), 6.94 (s, 1H), 6.78 (d, J=3.7 Hz, 1H), 6.76 (s, 1H), 6.70 (d, J=3.5 Hz, 1H), 6.57 (d, J=3.5 Hz, 1H), 6.35 (d, J=3.7 Hz, 1H), 6.28 (s, 1H), 3.91 (s, 3H), 3.89 (s, 3H), 1.40 (s, 9H), 1.13 (s, 9H), 1.11 (s, 9H).

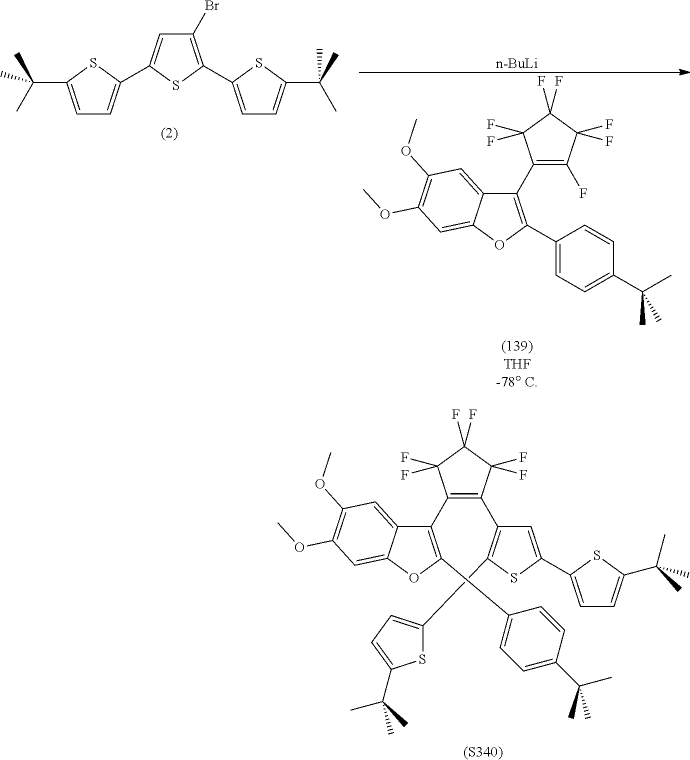

### Example 12: Preparation of Chromophore S373

To a stirred solution of 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)-5,6-dimethoxybenzofuran (4.54 g, 22.68 mmol) in acetonitrile (10 mL) iodotrimethylsilane (3.82 g, 4.54 mmol) was added. The solution was heated to reflux and stirred for 16 h. The reaction mixture was quenched with water and extracted with EtOAc (3×50 mL). The organic layer was washed with sodium thiosulfate (aqueous saturated), dried over magnesium sulfate and concentrated under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5,6-diol (3.5 g, 4.29 mmol, 95% yield) was purified by column chromatography.

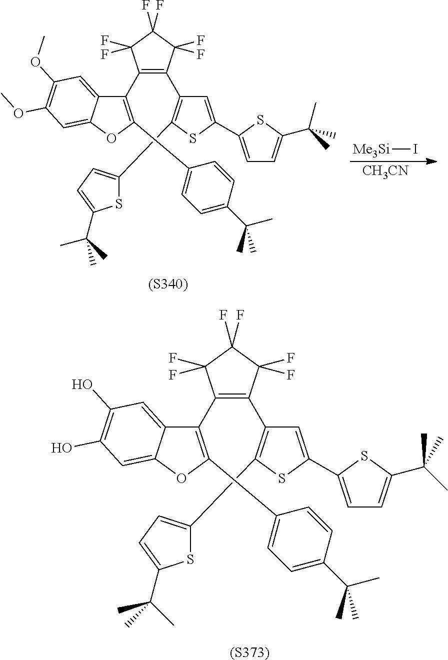

### Example 11: Preparation of Chromophore S364

To a solution of 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5,6-diol (3.85 g, 4.72 mmol), potassium carbonate (2.61 g, 18.90 mmol) and potassium iodide (0.314 g, 1.890 mmol) in acetone (40 ml), 1-bromo-2-methoxyethane (1.066 ml, 11.34 mmol) was added. The reaction mixture was heated to reflux and stirred for 16 h. Upon cooling to room temperature, the mixture was filtered through a frit and the filtrate was concentrated under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)-5,6-bis(2-methoxyethoxy)benzofuran (2.7 g, 2.90 mmol, 61.4% yield) was purified by column chromatography and recrystallized in methanol to afford a yellow crystalline solid. ¹H NMR (400 MHz, CDCl₃) δ 7.24-7.18 (m, 4H), 6.97 (s, 1H), 6.81 (s, 1H), 6.77 (d, J=3.7 Hz, 1H), 6.68 (d, J=3.7 Hz, 1H), 6.51 (d, J=3.5 Hz, 1H), 6.36-6.30 (m, 2H), 4.16 (t, J=4.9 Hz, 2H), 4.11 (t, J=4.6 Hz, 2H), 3.83-3.78 (m, 2H), 3.75 (t, J=4.8 Hz, 2H), 3.48 (s, 3H), 3.47 (s, 3H), 1.39 (s, 9H), 1.12 (s, 9H), 1.11 (s, 9H).

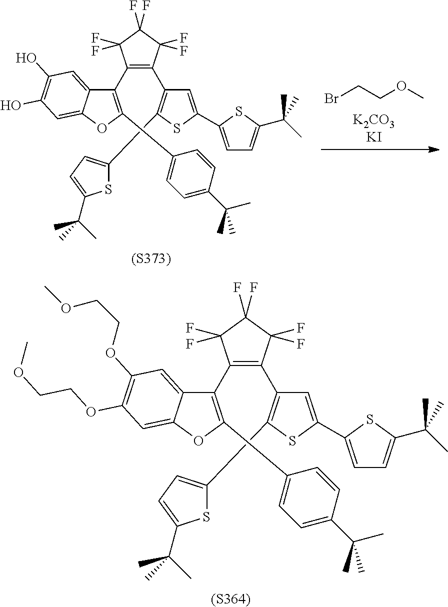

### Example 14: Preparation of Chromophore S374

To a solution of 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5,6-diol (2.4 g, 2.94 mmol), potassium carbonate (3.26 g, 23.56 mmol) and potassium iodide (0.196 g, 1.178 mmol) in acetone (25 ml), ethyl 4-bromobutanoate (3.37 ml, 23.56 mmol) was added. The mixture was heated to reflux for 72 h. Upon cooling to room temperature, the mixture was filtered through a frit and the filtrate was concentrated under reduced pressure. The product, diethyl 4,4′-((2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5,6-diyl)bis(oxy))dibutanoate (2.6374 g, 2.53 mmol, 86% yield) was purified by column chromatography and recrystallized in methanol to afford a yellow crystalline solid. ¹H NMR (400 MHz, CDCl₃) δ 7.25-7.20 (m, 2H), 7.20-7.16 (m, 2H), 6.91 (s, 1H), 6.77 (d, J=3.7 Hz, 1H), 6.74 (s, 1H), 6.68 (d, J=3.7 Hz, 1H), 6.52 (d, J=3.7 Hz, 1H), 6.31-6.29 (m, 2H), 4.18 (qd, J=7.2, 3.9 Hz, 4H), 4.01 (dt, J=19.4, 6.1 Hz, 4H), 2.56 (td, J=7.3, 2.5 Hz, 4H), 2.15 (dquin, J=13.7, 6.8, 6.8, 6.8, 6.8 Hz, 4H), 1.38 (s, 9H), 1.29 (td, J=7.2, 2.8 Hz, 6H), 1.10 (s, 9H). 1.11 (s, 9H).

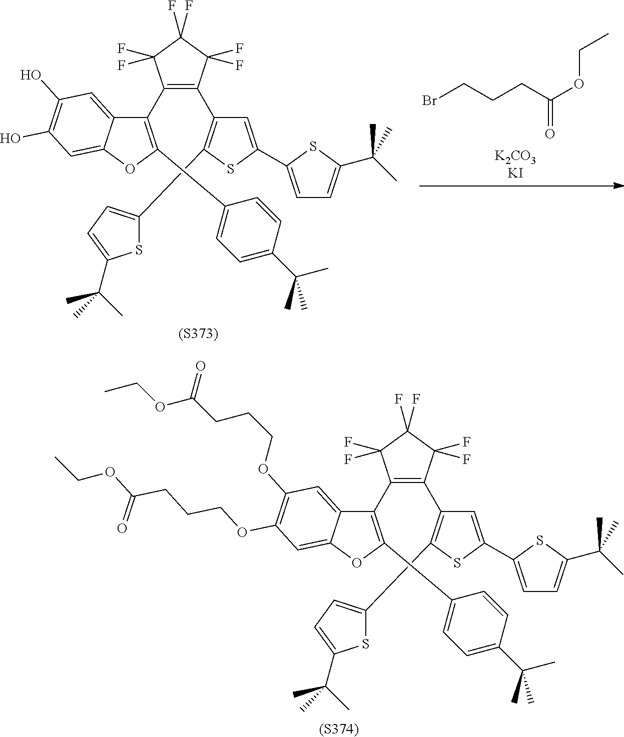

### Example 15: Preparation of Chromophore S367

To a solution of 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5,6-diol (3.4921 g, 4.28 mmol) and triethylamine (1.59 mL, 11.4 mmol) in dichloromethane (30 mL) at 0° C., acetic anhydride (1.08 mL, 11.4 mmol) was added dropwise. The solution was warmed to room temperature and stirred for 16 h. The mixture was quenched with aqueous hydrochloric acid (10 mL) and extracted with dichloromethane. The organic phase was dried over sodium sulfate and concentrated under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5,6-diyl diacetate (3.5 g, 3.89 mmol, 91 5 yield) was purified by column chromatography (hexanes/ethyl acetate) and recrystallized in methanol. ¹H NMR (400 MHz, CDCl₃) δ 7.29 (s, 1H), 7.26-7.24 (m, 2H), 7.24-7.18 (m, 2H), 7.08 (s, 1H), 6.76 (d, J=3.7 Hz, 1H), 6.68 (d, J=3.7 Hz, 1H), 6.38-6.32 (m, 1H), 6.32-6.29 (m, 1H), 6.20 (s, 1H), 2.32 (s, 3H), 2.31 (s, 3H), 1.38 (s, 9H), 1.22 (s, 9H), 1.10 (s, 9H).

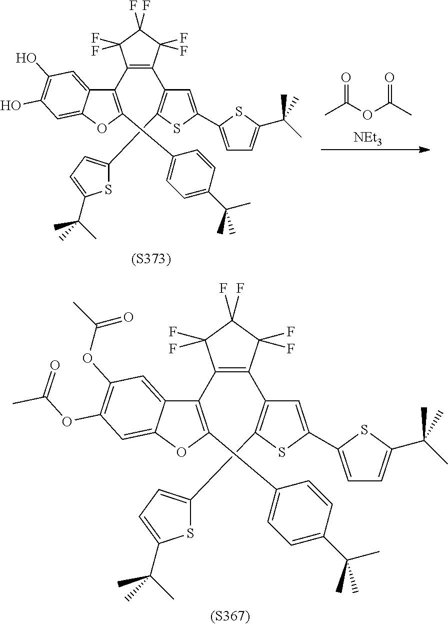

### Example 16: Preparation of Chromophore S343

A stirred solution of 3′-bromo-5,5″-di-tert-butyl-2,2′:5′,2″-terthiophene (4.6 g, 10.5 mmol) in anhydrous THF (30 mL) was cooled to −78° C. n-butyllithium (4.39 mL, 2.5 M in hexanes, 10.99 mmol) was added dropwise over 30 min. The solution stirred at −78° C. for 10 mins. 2-(4-(tert-butyl)phenyl)-3-(perfluorocyclopent-1-en-1-yl)benzofuran (4.86 g, 10.99 mmol) was added in one portion, the cold bath was removed and the mixture was warmed to room temperature and stirred for 3 h. Methanol (20 mL) was added to quench followed by concentration under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran (5.01 g, 6.40 mmol, 61.2% yield) was obtained by column purification (hexanes/ethyl acetate eluent) and precipitated from methanol to afford dark orange crystals. ¹H NMR (400 MHz, CDCl₃) δ 7.41-7.37 (m, 1H), 7.35 (d, J=7.8 Hz, 1H), 7.26 (s, 4H), 7.24-7.20 (m, 1H), 7.16-7.09 (m, 1H), 6.77 (d, J=3.7 Hz, 1H), 6.70 (d, J=3.5 Hz, 1H), 6.41 (d, J=3.7 Hz, 1H), 6.23-6.20 (m, 1H), 6.19 (d, J=3.7 Hz, 1H), 1.40 (s, 9H), 1.16 (s, 9H), 1.12 (s, 9H).

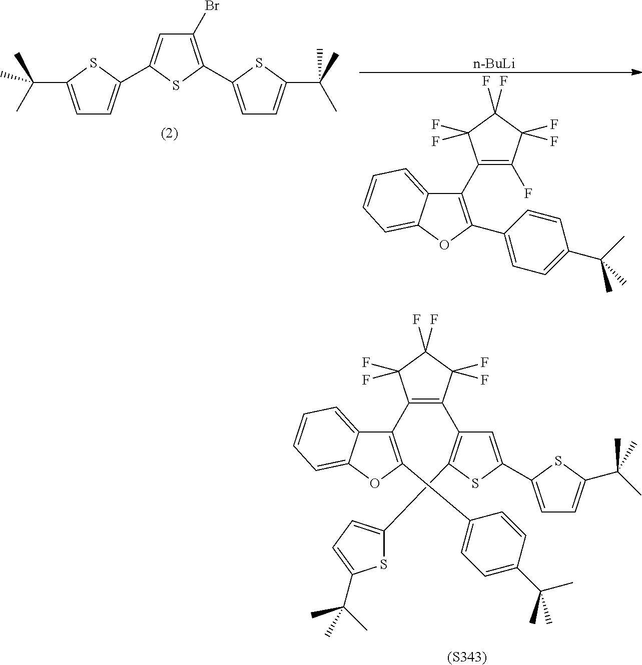

### Example 17: Preparation of Chromophore S344

A stirred solution of 3′-bromo-5,5″-di-tert-butyl-2,2′:5′,2″-terthiophene (16.37 g, 37.3 mmol) in anhydrous THF (200 mL) was cooled to −78° C. n-butyllithium (15.21 ml, 37.3 mmol) was added dropwise over 30 min. The solution stirred at −78° C. for 10 mins. 2-(4-(tert-butyl)phenyl)-5-methoxy-3-(perfluorocyclopent-1-en-1-yl)benzofuran (14.6669 g, 31.0 mmol) in THF (60 mL) was added in one portion, the cold bath was removed and the mixture was warmed to room temperature and stirred for 3 h. Methanol (20 mL) was added to quench followed by concentration under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)-5-methoxybenzofuran (13.1 g, 16.11 mmol, 51.9% yield) was purified by column chromatography (hexanes/ethyl acetate eluent) and precipitated in methanol. ¹H NMR (400 MHz, CDCl₃) δ 7.26-7.21 (m, 5H), 6.82 (d, J=2.5 Hz, 1H), 6.81-6.78 (m, 2H), 6.76 (d, J=3.7 Hz, 1H), 6.69 (d, J=3.7 Hz, 1H), 6.50 (d, J=3.7 Hz, 1H), 6.29 (d, J=3.7 Hz, 1H), 6.22 (s, 1H), 3.81 (s, 3H), 1.39 (s, 9H), 1.13 (s, 9H), 1.10 (s, 9H).

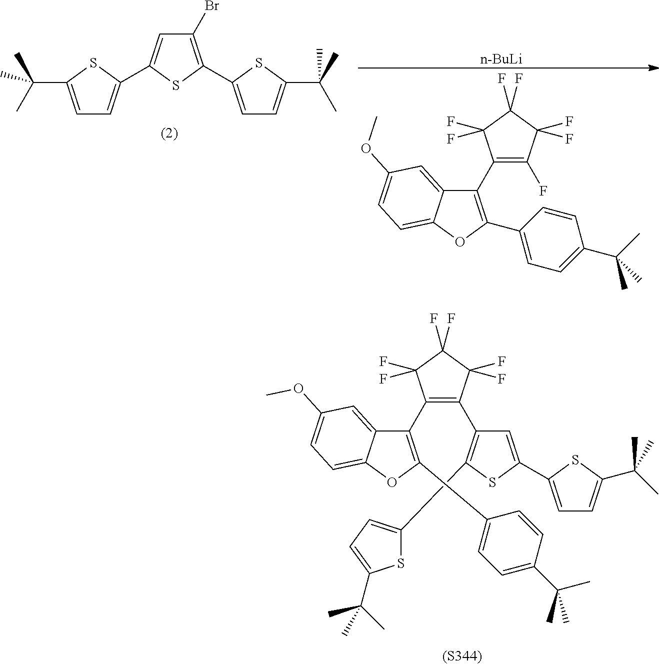

### Example 18: Preparation of Chromophore S362

A stirred solution of 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)-5-methoxybenzofuran (2.00 g, 2.460 mmol) in dichloromethane (20 mL) was cooled to 0° C. Boron tribromide (1.23 g, 4.92 mmol) was added and the mixture was stirred for 2 h at 0° C., warmed to room temperature and stirred for 16 h. Water was added (10 mL), the phases were separated and the aqueous phase was extracted with dichloromethane. The organic extracts were dried over magnesium sulfate and concentrated under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5-ol (1.7 g, 2.01 mmol, 86% yield) was used without further purification.

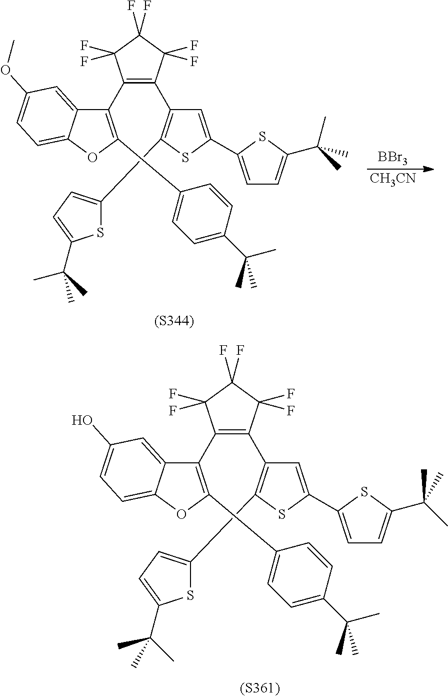

To a solution of 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)benzofuran-5-ol (3.4 g, 4.26 mmol), potassium carbonate (1.176 g, 8.51 mmol) and potassium iodide (0.141 g, 0.851 mmol) in acetone (100 ml), 2-(2-(2-ethoxyethoxy)ethoxy)ethyl 4-methylbenzenesulfonate (2.97 g, 8.94 mmol) was added. The mixture was heated to reflux for 16 h. Upon cooling to room temperature, the mixture was filtered through a frit and the filtrate was concentrated under reduced pressure. The product, (2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)-5-(2-(2-(2-ethoxyethoxy)ethoxy)ethoxy)benzofuran (2.8 g, 2.92 mmol, 68.6% yield) was purified by column chromatography (hexanes/ethyl acetate eluent) and recrystallized in methanol. ¹H NMR (400 MHz, CDCl₃) δ 7.22 (d, J=3.3 Hz, 4H), 6.85 (d, J=2.7 Hz, 1H), 6.82 (d, J=2.5 Hz, 1H), 6.78 (d, J=2.3 Hz, 1H), 6.76 (d, J=3.7 Hz, 1H), 6.68 (d, J=3.5 Hz, 1H), 6.48 (d, J=3.7 Hz, 1H), 6.28 (d, J=3.7 Hz, 1H), 6.24 (s, 1H), 4.10 (t, J=5.0 Hz, 2H), 3.90-3.84 (m, 2H), 3.80-3.75 (m, 2H), 3.74-3.67 (m, 4H), 3.60-3.56 (m, 2H), 3.40 (s, 3H), 1.38 (s, 9H), 1.13 (s, 9H), 1.10 (s, 9H).

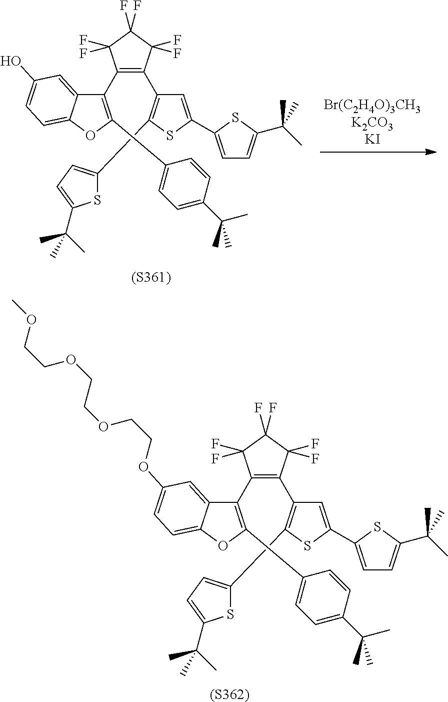

### Example 19: Preparation of Chromophore S345

A stirred solution of 3-bromo-2-(4-(tert-butyl)phenyl)-6-methoxybenzofuran (1.000 g, 2.78 mmol) in anhydrous THF (10 mL) and anhydrous 1,2-dimethoxyether (10 mL) was cooled to −78° C. n-butyllithium (1.169 ml, 2.5 M in hexanes, 2.92 mmol) was added dropwise over 30 min. The solution stirred at −78° C. for 20 mins. 5,5″-di-tert-butyl-3′-(perfluorocyclopent-1-en-1-yl)-2,2′:5′,2″-terthiophene (2.307 g, 4.18 mmol) in THF (10 mL) was added in one portion, the solution was warmed −35° C. and stirred for 3 h. Methanol (20 mL) was added to quench followed by concentration under reduced pressure. The product, 2-(4-(tert-butyl)phenyl)-3-(2-(5,5″-di-tert-butyl-[2,2′:5′,2″-terthiophen]-3′-yl)-3,3,4,4,5,5-hexafluorocyclopent-1-en-1-yl)-5-methoxybenzofuran (0.835 g, 1.027 mmol, 36.9% yield) was purified by column chromatography (hexanes/ethyl acetate eluent) and precipitated from methanol. ¹H NMR (400 MHz, CDCl₃) δ 7.25-7.15 (m, 4H), 6.89 (d, J=2.2 Hz, 1H), 6.77-6.71 (m, 2H), 6.67 (d, J=3.7 Hz, 1H), 6.43 (d, J=3.5 Hz, 1H), 6.24 (d, J=3.7 Hz, 1H), 6.21 (s, 1H), 3.82 (s, 3H), 1.37 (s, 9H), 1.15 (s, 9H), 1.09 (s, 9H).

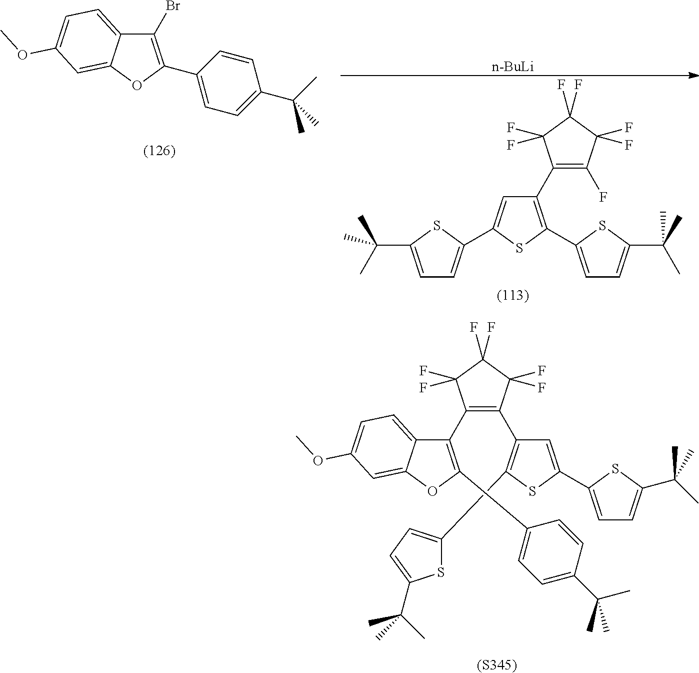

### Example 20: Preparation of Chromophore S253

A stirred solution of (3′-bromo-5″-(tert-butyl)-[2,2′:5′,2″-terthiophen]-5-yl)tris(3-methoxypropyl)silane (31.5 g, 50.0 mmol) in anhydrous THF (300 ml) was cooled to −78° C. n-butyllithium (22.01 ml, 2.5 M in hexanes, 55.0 mmol) was added dropwise over 30 min. The solution stirred at −78° C. for 10 mins. 2-(4-(tert-butyl)phenyl)-5,6-dimethoxy-3-(perfluorocyclopent-1-en-1-yl)benzofuran (32.7 g, 65.0 mmol) was added in one portion, the cold bath was removed and the mixture was warmed to room temperature and stirred for 3 h. Methanol (20 mL) was added to quench followed by concentration under reduced pressure. The product, (5″-(tert-butyl)-3′-(2-(2-(4-(tert-butyl)phenyl)-5,6-dimethoxybenzofuran-3-yl)-3,3,4,4,5,5-hexafluorocyclopent-1-en-1-yl)-[2,2′:5′,2″-terthiophen]-5-yl)tris(3-methoxypropyl)silane (30.9 g, 29.9 mmol, 59.8% yield) was obtained by column purification (dichloromethane/ethyl acetate eluent) and recrystallization from methanol to afford dark orange crystals. 1H NMR (400 MHz, CDCl₃) δ 7.26-7.18 (m, 4H), 6.92 (s, 1H), 6.81 (d, J=3.3 Hz, 1H), 6.78 (d, J=3.7 Hz, 1H), 6.76 (d, J=3.5 Hz, 1H), 6.71-6.67 (m, 2H), 6.32 (s, 1H), 3.90 (s, 3H), 3.84 (s, 3H), 3.38-3.31 (m, 15H), 1.59-1.49 (m, 6H), 1.39 (s, 9H), 1.12 (s, 9H), 0.69-0.61 (m, 6H).

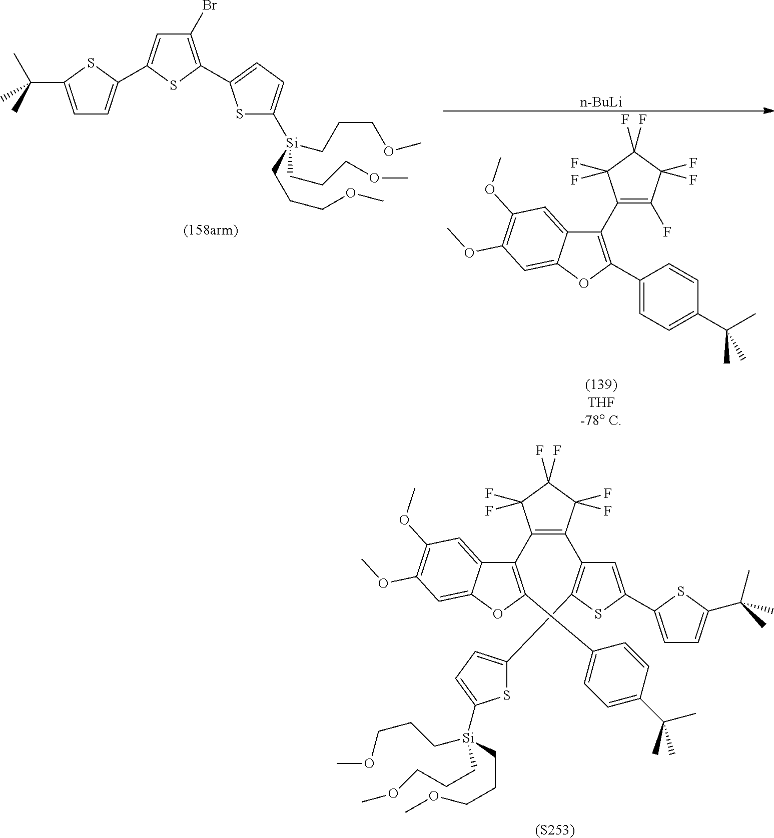

### Example 21: Preparation of Chromophore S378

A stirred solution of 3′-bromo-5,5″-di-tert-butyl-3-methyl-2,2′:5′,2″-terthiophene (3.2 g, 7.06 mmol) in anhydrous THF (45 ml) was cooled to −78° C. n-butyllithium (3.10 ml, 2.5 M in hexanes, 7.76 mmol) was added dropwise over 30 min. The mixture was stirred at −78° C. for 15 mins and a solution of 3-(2-([2,2′:5′,2″-terthiophen]-3′-yl)-3,3,4,4,5,5-hexafluorocyclopent-1-en-1-yl)-2-(4-(tert-butyl)phenyl)-5,6-dimethoxybenzofuran (4.61 g, 9.17 mmol in THF (10 mL) was added to the reaction mixture in one portion, the cold bath was removed and the mixture was warmed to room temperature and stirred for 2 h. Methanol (20 mL) was added to quench followed by concentration under reduced pressure. The product, 2-(4-(tert-butyl)phenyl)-3-(2-(5,5″-di-tert-butyl-3-methyl-[2,2′:5′,2″-terthiophen]-3′-yl)-3,3,4,4,5,5-hexafluorocyclopent-1-en-1-yl)-5,6-dimethoxybenzofuran (1.246 g, 1.454 mmol, 20.60% yield) was obtained by column purification (hexanes/ethyl acetate eluent) and precipitated from methanol. ¹H NMR (400 MHz, (CD₃)₂CO) δ 7.40-7.32 (m, 2H), 7.28-7.21 (m, 2H), 7.18 (s, 1H), 6.99 (d, J=3.7 Hz, 1H), 6.84 (d, J=3.7 Hz, 1H), 6.73 (s, 1H), 6.61 (s, 1H), 6.28 (s, 1H), 3.89 (s, 3H), 3.81 (s, 3H), 1.92 (s, 3H), 1.40 (s, 9H), 1.14 (s, 9H), 1.11 (s, 9H).

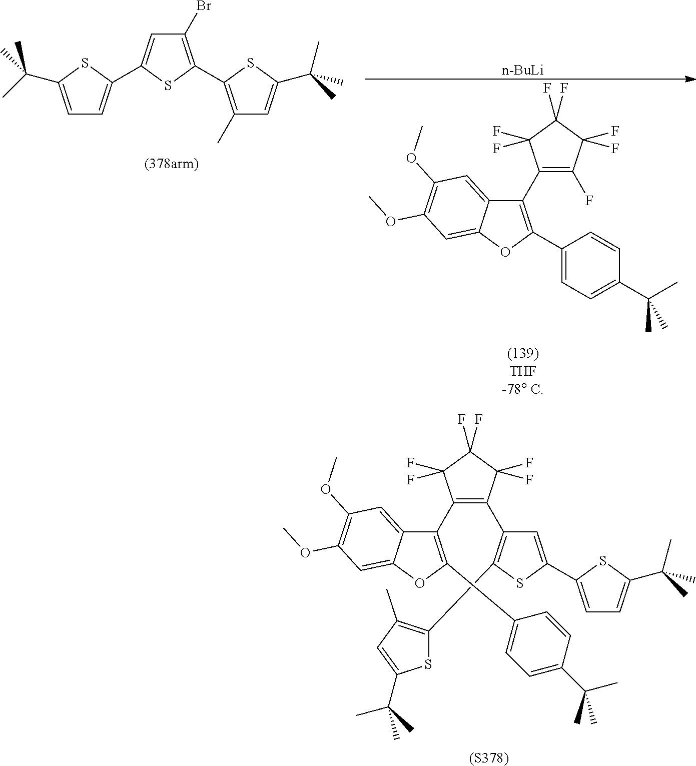

### Example 22: Preparation of Chromophore S383

To a stirred solution of 2-(4-(tert-butyl)phenyl)-3-(2-(5,5″-di-tert-butyl-3-methyl-[2,2′:5′,2″-terthiophen]-3′-yl)-3,3,4,4,5,5-hexafluorocyclopent-1-en-1-yl)-5,6-dimethoxybenzofuran (1.1 g, 1.283 mmol) (S378) in acetonitrile (10 ml), iodotrimethylsilane (0.913 ml, 6.42 mmol) was added. The reaction mixture was heated to reflux and stirred 16 h. Water (100 mL) was added and the mixture was extracted with ethyl acetate (3×200 mL). The combined organic extracted were treated with a saturated solution of sodium thiosulfate (100 mL), separated and dried over magnesium sulfate. The crude material was concentrated in vacuo. The product, 2-(4-(tert-butyl)phenyl)-3-(2-(5,5″-di-tert-butyl-3-methyl-[2,2′:5′,2″-terthiophen]-3′-yl)-3,3,4,4,5,5-hexafluorocyclopent-1-en-1-yl)benzofuran-5,6-diol (0.901 g, 1.087 mmol, 85% yield) was purified by column chromatography (hexanes/ethyl acetate eluent) and isolated as a yellow/green solid.

A solution of 2-(4-(tert-butyl)phenyl)-3-(2-(5,5″-di-tert-butyl-3-methyl-[2,2′:5′,2″-terthiophen]-3′-yl)-3,3,4,4,5,5-hexafluorocyclopent-1-en-1-yl)benzofuran-5,6-diol (0.9 g, 1.086 mmol), potassium carbonate (1.200 g, 8.69 mmol) and potassium iodide (0.144 g, 0.869 mmol) in acetone (10 ml), ethyl 4-bromobutanoate (1.243 ml, 8.69 mmol) was added. The reaction mixture was heated to reflux and stirred for 16 h. Upon cooling to room temperature, the mixture was filtered through a frit and the filtrate was concentrated under reduced pressure. The product, diethyl 4,4′-((2-(4-(tert-butyl)phenyl)-3-(2-(5,5″-di-tert-butyl-3-methyl-[2,2′:5′,2″-terthiophen]-3′-yl)-3,3,4,4,5,5-hexafluorocyclopent-1-en-1-yl)benzofuran-5,6-diyl)bis(oxy))dibutanoate (0.73 g, 0.690 mmol, 63.6% yield) was purified by column chromatography (hexanes/ethyl acetate eluent) and recrystallized in methanol to afford a yellow crystalline solid. ¹H NMR (400 MHz, (CD₃)₂CO) δ 7.40-7.32 (m, 2H), 7.27-7.20 (m, 2H), 7.19 (s, 1H), 7.02 (d, J=3.7 Hz, 1H), 6.84 (d, J=3.7 Hz, 1H), 6.73 (s, 1H), 6.69 (s, 1H), 6.28 (s, 1H), 4.22-4.09 (m, 4H), 3.87-4.09 (m, 4H), 2.58 (t, J=7.3 Hz, 2H), 2.56-2.45 (m, 2H), 2.19-2.09 (m, 2H), 1.91 (s, 3H), 1.40 (s, 9H), 1.24 (td, J=7.1, 2.5 Hz, 6H), 1.16 (s, 9H), 1.12 (s, 9H).

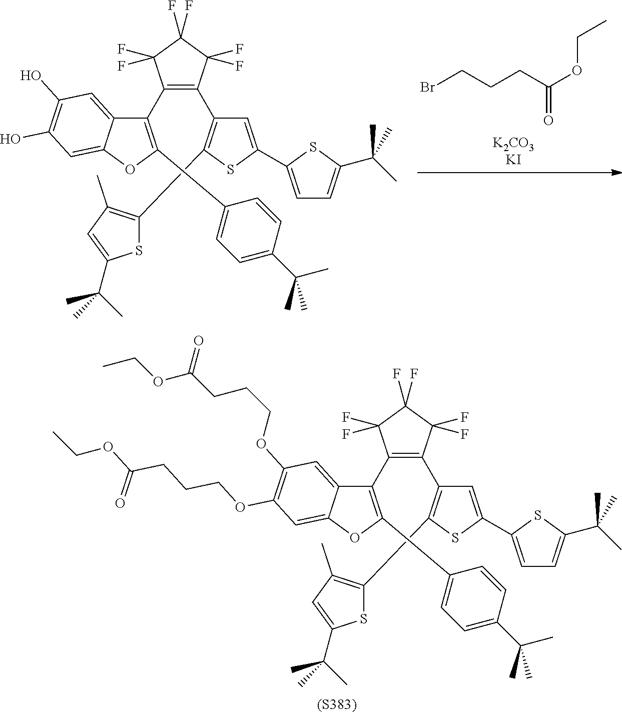

### Example 23: Preparation of Chromophore S377

A stirred solution of 3′-bromo-4,4″-di-tert-butyl-2,2′:5′,2″-terthiophene (20.00 g, 45.5 mmol) in anhydrous THF (200 ml) at −78° C., n-butyllithium (18.20 ml, 2.5 M in hexanes, 45.5 mmol) was added dropwise over 30 min. The solution was stirred at −78° C. for an additional 20 mins and a solution of 2-(4-(tert-butyl)phenyl)-5,6-dimethoxy-3-(perfluorocyclopent-1-en-1-yl)benzofuran in THF (50 ml) was added in one portion. The cold bath was removed, the mixture was warmed to room temperature over 30 minutes and stirred for an additional 2 h. Methanol (20 mL) was added to quench the remaining lithiate and the mixture was concentrated under reduced pressure. The product, 2-(4-(tert-butyl)phenyl)-3-(2-(4,4″-di-tert-butyl-[2,2′:5′,2″-terthiophen]-3′-yl)-3,3,4,4,5,5-hexafluorocyclopent-1-en-1-yl)-5,6-dimethoxybenzofuran (14.1 g, 16.73 mmol, 55.1% yield) was purified by column chromatography (hexanes/ethyl acetate eluent) and precipitated from methanol to yield a yellow solid. ¹H NMR (400 MHz, CDCl₃) δ 7.26-7.22 (m, 2H), 7.22-7.18 (m, 2H), 6.93 (d, J=1.6 Hz, 1H), 6.91 (s, 1H), 6.84 (d, J=1.6 Hz, 1H), 6.76 (s, 1H), 6.56-6.53 (m, 2H), 6.24 (s, 1H), 3.90 (s, 3H), 3.88 (s, 3H), 1.30 (s, 9 H), 1.09 (s, 9H), 1.08 (s, 9H).

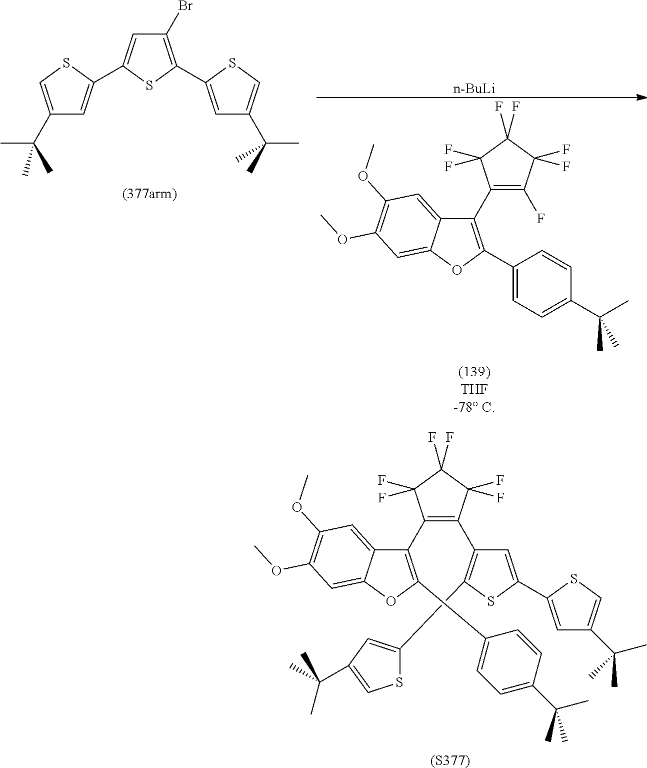

### Example 24: Preparation of Chromophore S379

A stirred solution of 3′-bromo-5,5″-di-tert-butyl-3,3″-dimethyl-2,2′:5′,2″-terthiophene (10.9639 g, 23.45 mmol) in anhydrous THF (110 ml) was cooled to −78° C. n-butyllithium (9.57 ml, 2.5 M in hexanes, 23.45 mmol) was added dropwise over 30 min. The mixture was stirred at −78° C. for 15 mins and a solution of 3-(2-([2,2′:5′,2″-terthiophen]-3′-yl)-3,3,4,4,5,5-hexafluorocyclopent-1-en-1-yl)-2-(4-(tert-butyl)phenyl)-5,6-dimethoxybenzofuran (9.82 g, 19.54 mmol) in THF (10 mL) was added to the reaction mixture in one portion, the cold bath was removed and the mixture was warmed to room temperature and stirred for 2 h. Methanol (20 mL) was added to quench followed by concentration under reduced pressure. The product, 2-(4-(tert-butyl)phenyl)-3-(2-(5,5″-di-tert-butyl-3,3″-dimethyl-[2,2′:5′,2″-terthiophen]-3′-yl)-3,3,4,4,5,5-hexafluorocyclopent-1-en-1-yl)-5,6-dimethoxybenzofuran (7.1 g, 8.15 mmol, 41.7% yield) was obtained by column purification (hexanes/ethyl acetate eluent) and precipitated from methanol.

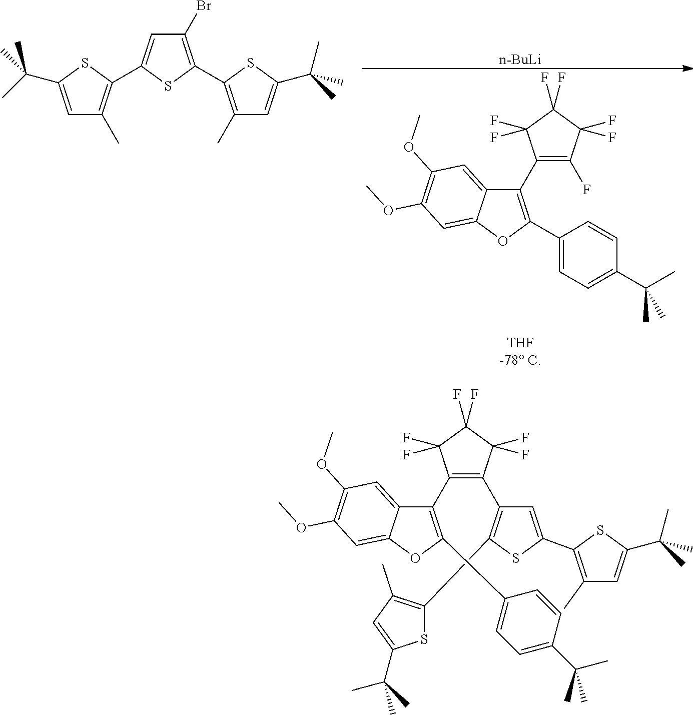

### Example 25: Preparation of Chromophore S384

To a stirred solution of 2-(4-(tert-butyl)phenyl)-3-(2-(5,5″-di-tert-butyl-3,3″-dimethyl-[2,2′:5′,2″-terthiophen]-3′-yl)-3,3,4,4,5,5-hexafluorocyclopent-1-en-1-yl)-5,6-dimethoxybenzofuran (5.4578 g, 6.27 mmol) (S379) in acetonitrile (32 ml), iodotrimethylsilane (4.46 ml, 31.3 mmol) was added. The reaction mixture was heated to reflux and stirred 16 h. Water (100 mL) was added and the mixture was extracted with ethyl acetate (3×200 mL). The combined organic extracts were treated with a saturated solution of sodium thiosulfate (100 mL), separated and dried over magnesium sulfate. The crude material was concentrated in vacuo. The product, 2-(4-(tert-butyl)phenyl)-3-(2-(5,5″-di-tert-butyl-3,3″-dimethyl-[2,2′:5′,2″-terthiophen]-3′-yl)-3,3,4,4,5,5-hexafluorocyclopent-1-en-1-yl)benzofuran-5,6-diol (4.25 g, 5.04 mmol, 80% yield) was purified by column chromatography (hexanes/ethyl acetate eluent) and isolated as a yellow/green solid.

A solution of 2-(4-(tert-butyl)phenyl)-3-(2-(5,5″-di-tert-butyl-3,3″-dimethyl-[2,2′:5′,2″-terthiophen]-3′-yl)-3,3,4,4,5,5-hexafluorocyclopent-1-en-1-yl)benzofuran-5,6-diol (4.2455 g, 5.04 mmol), potassium carbonate (5.57 g, 40.3 mmol) and potassium iodide (0.334 g, 2.014 mmol) in acetone (45 ml), ethyl 4-bromobutanoate (5.77 ml, 40.3 mmol) was added. The reaction mixture was heated to reflux and stirred for 16 h. Upon cooling to room temperature, the mixture was filtered through a frit and the filtrate was concentrated under reduced pressure. The product, diethyl 4,4′-((2-(4-(tert-butyl)phenyl)-3-(2-(5,5″-di-tert-butyl-3,3″-dimethyl-[2,2′:5′,2″-terthiophen]-3′-yl)-3,3,4,4,5,5-hexafluorocyclopent-1-en-1-yl)benzofuran-5,6-diyl)bis(oxy))dibutanoate (4.210 g, 3.93 mmol, 78% yield) was purified by column chromatography (hexanes/ethyl acetate eluent) and recrystallized in methanol to afford a yellow crystalline solid. ¹H NMR (400 MHz, CDCl₃) δ 7.26-7.23 (m, 2H), 7.22-7.17 (m, 2H), 6.95 (s, 1H), 6.68 (s, 1H), 6.56 (s, 1H), 6.45 (s, 1H), 6.11 (s, 1H), 4.18 (qd, J=7.2, 1.0 Hz, 4H), 4.05 (t, J=6.2 Hz, 2H), 3.96 (d, J=16.2 Hz, 2H), 2.56 (dt, J=14.4, 7.4 Hz, 4H), 2.24 (s, 3H), 2.22-2.07 (m, 4H), 1.86 (s, 3H), 1.37 (s, 9H), 1.29 (t, J=7.1 Hz, 3H), 1.29 (t, J=7.1 Hz, 3H), 1.11 (s, 9H), 1.11 (s, 9H).

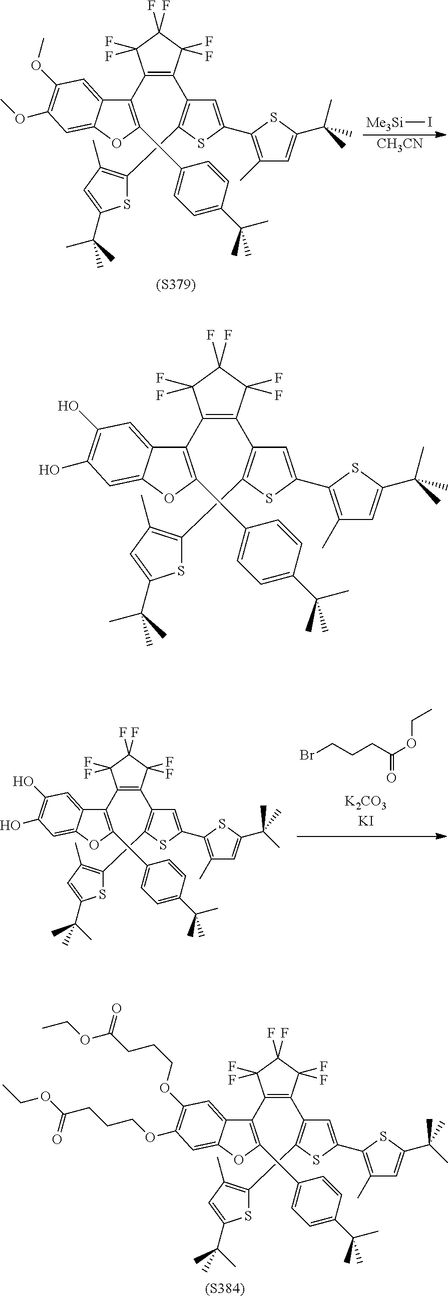

### Example 26: Preparation of Chromophore S393

To a stirred solution of 2-(4-(tert-butyl)phenyl)-3-(2-(4,5″-di-tert-butyl-[2,2′:5′,2″-terthiophen]-3′-yl)-3,3,4,4,5,5-hexafluorocyclopent-1-en-1-yl)-5,6-dimethoxybenzofuran (13.77 g, 16.33 mmol) in 1,2-dichloroethane (100 ml), iodotrimethylsilane (11.62 ml, 82 mmol) was added. The reaction mixture was heated to reflux and stirred 16 h. Water (100 mL) was added and the mixture was extracted with ethyl acetate (3×200 mL). The combined organic extracted were treated with a saturated solution of sodium thiosulfate (100 mL), separated and dried over magnesium sulfate. The crude material was concentrated in vacuo. The product, 2-(4-(tert-butyl)phenyl)-3-(2-(4,5″-di-tert-butyl-[2,2′:5′,2″-terthiophen]-3′-yl)-3,3,4,4,5,5-hexafluorocyclopent-1-en-1-yl)benzofuran-5,6-diol (12.2 g, 14.97 mmol, 92% yield) was purified by column chromatography (hexanes/ethyl acetate eluent) and isolated as a yellow/green solid.

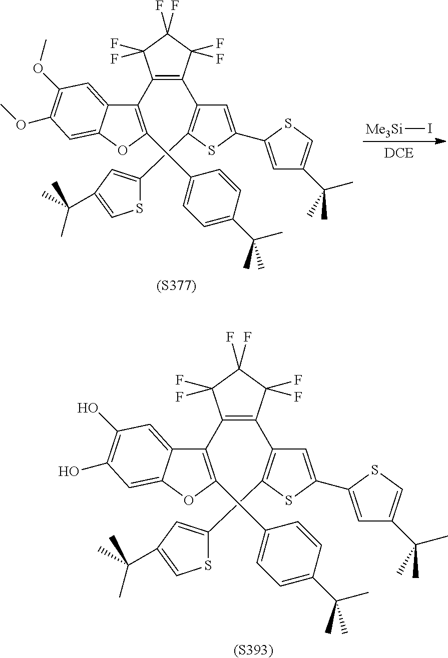

### Example 27: Preparation of Chromophore S386

A solution of 2-(4-(tert-butyl)phenyl)-3-(2-(4,4″-di-tert-butyl-[2,2′:5′,2″-terthiophen]-3′-yl)-3,3,4,4,5,5-hexafluorocyclopent-1-en-1-yl)benzofuran-5,6-diol (1.600 g, 1.963 mmol), potassium carbonate (2.171 g, 15.71 mmol) and potassium iodide (0.261 g, 1.571 mmol) in acetone (13 ml), ethyl 4-bromobutanoate (2.248 ml, 15.71 mmol) was added. The reaction mixture was heated to reflux and stirred for 16 h. Upon cooling to room temperature, the mixture was filtered through a frit and the filtrate was concentrated under reduced pressure. The product, diethyl 4,4′-((2-(4-(tert-butyl)phenyl)-3-(2-(4,4″-di-tert-butyl-[2,2′:5′,2″-terthiophen]-3′-yl)-3,3,4,4,5,5-hexafluorocyclopent-1-en-1-yl)benzofuran-5,6-diyl)bis(oxy))dibutanoate (1.5 g, 1.438 mmol, 73.2% yield) was purified by column chromatography (hexanes/ethyl acetate eluent) and recrystallized in methanol to afford a yellow crystalline solid. ¹H NMR (400 MHz, CDCl₃) δ 7.21 (q, J=8.6 Hz, 4H), 6.93 (d, J=1.6 Hz, 1H), 6.91 (s, 1H), 6.83 (d, J=1.4 Hz, 1H), 6.75 (s, 1H), 6.54 (s, 2H), 6.26 (s, 1H), 4.18 (qd, J=7.2, 4.7 Hz, 4H), 4.04 (t, J=6.2 Hz, 2H), 3.99 (t, J=6.2 Hz, 2H), 2.57 (t, J=7.2 Hz, 4H), 2.15 (dquin, J=14.2, 6.8, 6.8, 6.8, 6.8 Hz, 4H), 1.33-1.24 (m, 15H), 1.09 (s, 9H), 1.08 (s, 9H).

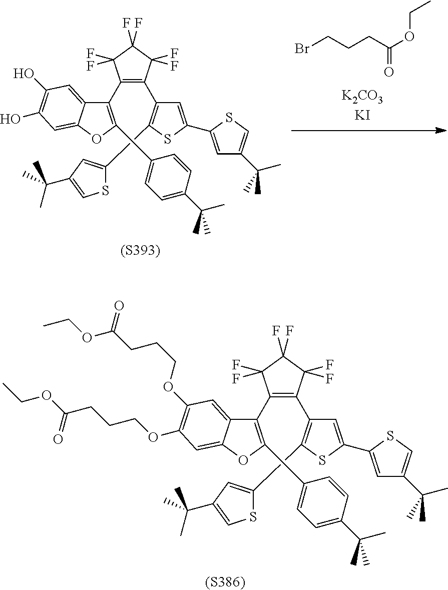

### Example 28: Preparation of Chromophore S392

A solution of 2-(4-(tert-butyl)phenyl)-3-(2-(4,5″-di-tert-butyl-[2,2′:5′,2″-terthiophen]-3′-yl)-3,3,4,4,5,5-hexafluorocyclopent-1-en-1-yl)benzofuran-5,6-diol (2.000 g, 2.454 mmol), potassium carbonate (2.71 g, 19.63 mmol) and potassium iodide (0.326 g, 1.963 mmol) in acetone (16 ml), 1-bromo-3-methoxypropane (3.00 g, 19.63 mmol) was added. The reaction mixture was heated to reflux and stirred for 16 h. Upon cooling to room temperature, the mixture was filtered through a frit and the filtrate was concentrated under reduced pressure. The product, 2-(4-(tert-butyl)phenyl)-3-(2-(4,5″-di-tert-butyl-[2,2′:5′,2″-terthiophen]-3′-yl)-3,3,4,4,5,5-hexafluorocyclopent-1-en-1-yl)-5,6-bis(3-methoxypropoxy)benzofuran (1.7 g, 1.772 mmol, 72.2% yield) was purified by column chromatography (hexanes/ethyl acetate eluent) and recrystallized in methanol to afford a yellow crystalline solid. ¹H NMR (400 MHz, CDCl₃) δ 7.26-7.17 (m, 4H), 6.97-6.91 (m, 2H), 6.85 (d, J=1.4 Hz, 1H), 6.79 (s, 1H), 6.57 (d, J=2.9 Hz, 2H), 6.27 (s, 1H), 4.08 (m, J=14.9, 6.1, 6.1 Hz, 4H), 3.68-3.58 (m, 4H), 3.41 (s, 3H), 3.40 (s, 3H), 2.17-2.04 (m, 4H), 1.31 (s, 9H), 1.10 (s, 18H).

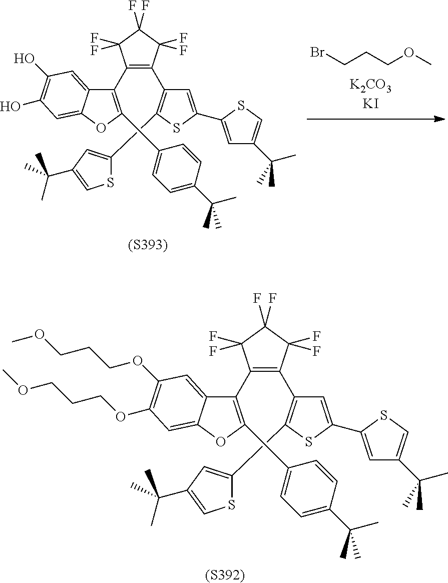

### Example 29: Preparation of Chromophore S396

A solution of 2-(4-(tert-butyl)phenyl)-3-(2-(4,5″-di-tert-butyl-[2,2′:5′,2″-terthiophen]-3′-yl)-3,3,4,4,5,5-hexafluorocyclopent-1-en-1-yl)benzofuran-5,6-diol (2.000 g, 2.454 mmol) and triethylamine (1.821 ml, 12.27 mmol) in dichloromethane (13 mL) at 0° C., acetic anhydride (1.08 mL, 11.4 mmol) was added dropwise. The solution was warmed to room temperature and stirred for 16 h. The mixture was quenched with aqueous hydrochloric acid (10 mL) and extracted with dichloromethane. The organic phase was dried over sodium sulfate and concentrated under reduced pressure. The product, 2-(4-(tert-butyl)phenyl)-3-(2-(4,4″-di-tert-butyl-[2,2′:5′,2″-terthiophen]-3′-yl)-3,3,4,4,5,5-hexafluorocyclopent-1-en-1-yl)benzofuran-5,6-diyl diacetate (0.80 g, 0.89 mmol, 36.3% yield) was purified by column chromatography (hexanes/ethyl acetate eluent) and recrystallized in methanol to afford a yellow crystalline solid. ¹H NMR (400 MHz, CDCl₃) δ 7.26-7.19 (m, 4H), 7.00 (s, 1H), 6.93 (d, J=1.6 Hz, 1H), 6.84 (d, J=1.6 Hz, 1H), 6.65 (d, J=1.6 Hz, 1H), 6.59 (d, J=1.6 Hz, 1H), 6.19 (s, 1H), 2.33 (s, 3H), 2.32 (s, 3H), 1.30 (s, 9H), 1.18 (s, 9H), 1.08 (s, 9H).

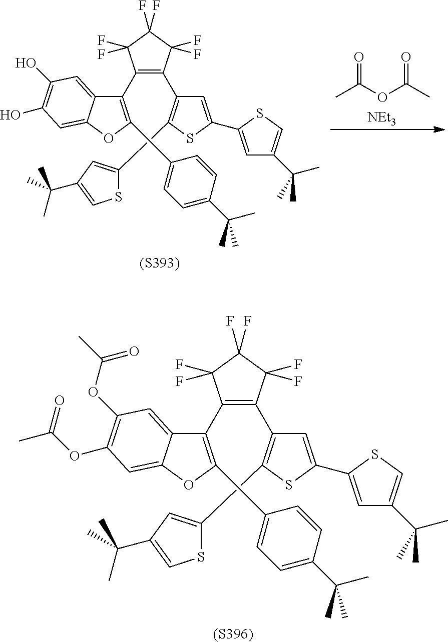

### Example 30: Preparation of Chromophore S398

A solution of 2-(4-(tert-butyl)phenyl)-3-(2-(4,5″-di-tert-butyl-[2,2′:5′,2″-terthiophen]-3′-yl)-3,3,4,4,5,5-hexafluorocyclopent-1-en-1-yl)benzofuran-5,6-diol (7.50 g, 9.20 mmol) and potassium carbonate (10.18 g, 74.00 mmol) in acetone (60 ml), 2-(2,5-dioxopyrrolidin-1-yl)ethyl 4-toluenesulfonate (21.60 g, 74.00 mmol) was added. The reaction mixture was heated to reflux and stirred for 24 h. Upon cooling to room temperature, the mixture was filtered through a frit and the filtrate was concentrated under reduced pressure. The product, 2-(4-(tert-butyl)phenyl)-3-(2-(4,5″-di-tert-butyl-[2,2′:5′,2″-terthiophen]-3′-yl)-3,3,4,4,5,5-hexafluorocyclopent-1-en-1-yl)-5,6-bis(3-methoxypropoxy)benzofuran (4.5 g, 4.22 mmol, 46% yield) was purified by column chromatography (hexanes/ethyl acetate eluent) and recrystallized in methanol to afford a yellow crystalline solid. ¹H NMR (400 MHz, CDCl₃) δ 7.19-7.16 (m, 2H) 7.24-7.20 (m, 2H), 6.93 (d, J=1.6 Hz, 1H), 6.89 (s, 1H), 6.83 (d, J=1.6 Hz, 1H), 6.72 (s, 1H), 6.56 (d, J=1.6 Hz, 1H), 6.54 (d, J=1.4 Hz, 1H), 6.23 (s, 1H), 4.18-4.13 (m, 2H), 4.13-4.08 (m, 2H), 3.96 (m, 4H), 2.77 (s, 4H), 2.77 (s, 4H), 1.29 (s, 9H), 1.08 (s, 9H), 1.07 (s, 9H).

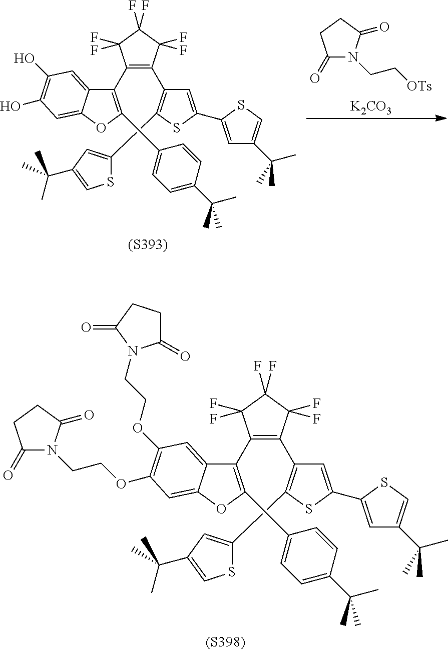

### Example 31: Preparation of Chromophore S381

A stirred solution of 2-[3-bromo-5-(4,4,7,7-tetramethyl-4,5,6,7-tetrahydro-1-benzothiophen-2-yl)thiophen-2-yl]-4,4,7,7-tetramethyl-4,5,6,7-tetrahydro-1-benzothiophene) (35.765 g, 0.065 mol) in anhydrous THF (360 mL) at −78° C., n-butyllithium (26.100 mL, 2.5 M in hexanes, 0.065 mol) was added dropwise over 20 min. The solution was stirred at −78° C. for an additional 20 mins and a solution of 2-(4-(tert-butyl)phenyl)-5,6-dimethoxy-3-(perfluorocyclopent-1-en-1-yl)benzofuran (23.530 g, 0.047 mol) in THF (70 mL) was added in one portion. The cold bath was removed, the mixture was warmed to room temperature over 30 minutes and stirred for an additional 2 h. Methanol (20 mL) was added to quench the remaining lithiate and the mixture was concentrated under reduced pressure. The product, 3-{2-[2,5-bis(4,4,7,7-tetramethyl-4,5,6,7-tetrahydro-1-benzothiophen-2-yl)thiophen-3-yl]-3,3,4,4,5,5-hexafluorocyclopent-1-en-1-yl}-2-(4-tert-butylphenyl)-5,6-dimethoxy-1-benzothiophene) (35.500 g, 0.037 mol, 80.0%) was obtained by column purification (hexanes/ethyl acetate eluent) and precipitation from methanol. ¹H NMR (400 MHz, CDCl₃) δ 7.26-7.22 (m, 2H), 7.22-7.12 (m, 2H), 6.96 (s, 1H), 6.94 (s, 1H), 6.70 (s, 1 H), 6.42 (s, 1H), 6.20 (s, 1H), 3.90 (s, 3H), 3.89 (s, 3H), 1.82-1.61 (m, 4H), 1.49 (d, J=7.8 Hz, 4H), 1.33 (s, 6H), 1.23 (s, 6H), 0.90-1.18 (m, 21H).

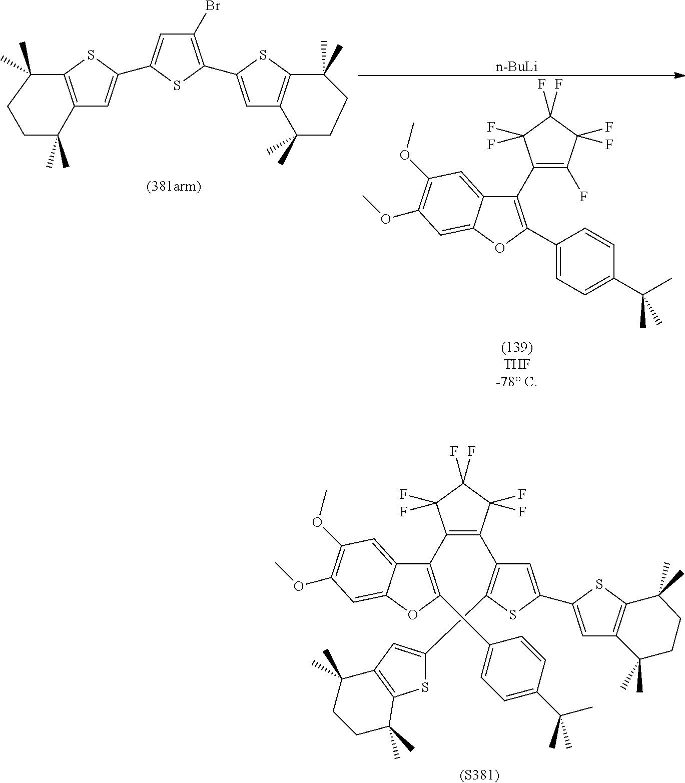

### Example 32: Preparation of Chromophore S391

To a stirred solution of 3-{2-[2,5-bis(4,4,7,7-tetramethyl-4,5,6,7-tetrahydro-1-benzothiophen-2-yl)thiophen-3-yl]-3,3,4,4,5,5-hexafluorocyclopent-1-en-1-yl}-2-(4-tert-butylphenyl)-5,6-dimethoxy-1-benzothiophene) (47.100 g, 0.050 mol) in 1,2-dichloroethane (300 ml), iodotrimethylsilane (35.200 mL, 0.248 mol) was added. The reaction mixture was heated to reflux and stirred 16 h. Water (100 mL) was added and the mixture was extracted with ethyl acetate (3×200 mL). The combined organic extracted were treated with a saturated solution of sodium thiosulfate (100 mL), separated and dried over magnesium sulfate. The crude material was concentrated in vacuo. The product, 3-{2-[2,5-bis(4,4,7,7-tetramethyl-4,5,6,7-tetrahydro-1-benzothiophen-2-yl)thiophen-3-yl]-3,3,4,4,5,5-hexafluorocyclopent-1-en-1-yl}-2-(4-tert-butylphenyl)-1-benzofuran-5,6-diol) (30.000 g, 0.032 mol, 65.6%) was purified by column chromatography (hexanes/ethyl acetate eluent) and isolated as a yellow/green solid.

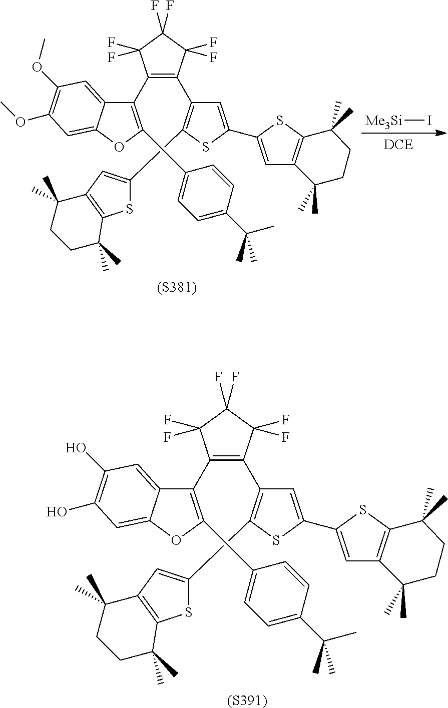

### Example 33: Preparation of Chromophore S387

A solution of 3-{2-[2,5-bis(4,4,7,7-tetramethyl-4,5,6,7-tetrahydro-1-benzothiophen-2-yl)thiophen-3-yl]-3,3,4,4,5,5-hexafluorocyclopent-1-en-1-yl}-2-(4-tert-butylphenyl)-1-benzofuran-5,6-diol) (15.000 g, 0.016 mol), potassium carbonate (17.965 g, 0.130 mol) and potassium iodide (2.697 g, 0.016 mol) in acetone (160 ml), ethyl 4-bromobutanoate (18.600 mL, 0.130 mol) was added. The reaction mixture was heated to reflux and stirred for 16 h. Upon cooling to room temperature, the mixture was filtered through a frit and the filtrate was concentrated under reduced pressure. The product, diethyl ethyl 4-[(3-{2-[2,5-bis(4,4,7,7-tetramethyl-4,5,6,7-tetrahydro-1-benzothiophen-2-yl)thiophen-3-yl]-3,3,4,4,5,5-hexafluorocyclopent-1-en-1-yl}-2-(4-tert-butylphenyl)-6-(4-ethoxy-4-oxobutoxy)-1-benzofuran-5-yl)oxy]butanoate) (10.800 g, 0.009 mol, 57.7%) was purified by column chromatography (hexanes/ethyl acetate eluent) and recrystallized in methanol to afford a yellow crystalline solid. ¹H NMR (400 MHz, CDCl₃) δ 7.24-7.20 (m, 2H), 7.19-7.15 (m, 2H), 6.91 (s, 1H), 6.91 (s, 1H), 6.68 (s, 1H), 6.38 (s, 1H), 6.17 (s, 1H), 4.17 (q, J=7.2 Hz, 4H), 4.00 (t, J=6.1 Hz, 4H), 2.59-2.52 (m, 4H), 2.21-2.04 (m, 4H), 1.75-1.62 (m, 4H), 1.55-1.44 (m, 4H), 1.31 (s, 6H), 1.28 (t, J=7.1 Hz, 6H), 1.21 (s, 6H), 0.87-1.17 (m, 21H).

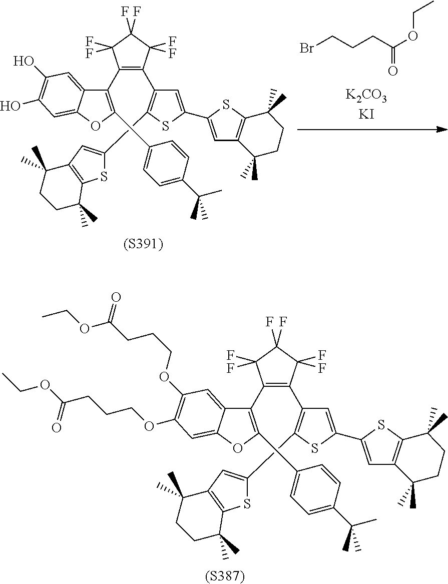

### Example 34: Preparation of Chromophore S388

A solution of 3-{2-[2,5-bis(4,4,7,7-tetramethyl-4,5,6,7-tetrahydro-1-benzothiophen-2-yl)thiophen-3-yl]-3,3,4,4,5,5-hexafluorocyclopent-1-en-1-yl}-2-(4-tert-butylphenyl)-1-benzofuran-5,6-diol) (5.00 g, 0.005 mol) and potassium carbonate (5.99 g, 0.043) in acetone (40 mL), 3-methoxypropyl 4-methylbenzenesulfonate (12.4 g, 0.043 mol) was added. The reaction mixture was heated to reflux and stirred for 16 h. Upon cooling to room temperature, the mixture was filtered through a frit and the filtrate was concentrated under reduced pressure. The product, 3-(2-(2,5-bis(4,4,7,7-tetramethyl-4,5,6,7-tetrahydrobenzo[b]thiophen-2-yl)thiophen-3-yl)-3,3,4,4,5,5-hexafluorocyclopent-1-en-1-yl)-2-(4-(tert-butyl)phenyl)-5,6-bis(3-methoxypropoxy)benzofuran (5.5 g, 0.005 mol, 95.1%) was purified by column chromatography (hexanes/ethyl acetate eluent) and recrystallized in methanol to afford a yellow crystalline solid. ¹H NMR (400 MHz, CDCl₃) δ 7.24-7.19 (m, 2H), 7.20-7.14 (m, 2H), 6.93 (s, 1H), 6.92 (s, 1H), 6.68 (s, 1H), 6.39 (s, 1H), 6.16 (s, 1H), 4.06 (td, J=6.2, 3.5 Hz, 4H), 3.63-3.57 (m, 4H), 3.38 (s, 3H), 3.37 (s, 3H), 2.15-1.99 (m, 4H), 1.75-1.60 (m, 4H), 1.50 (m, 4H), 1.31 (s, 6H), 1.21 (s, 6H), 0.83-1.17 (m, 21H).

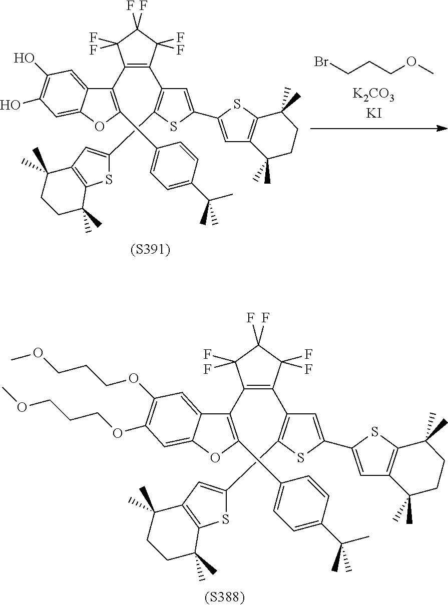

### Example 35: Preparation of Chromophore S390

A solution of 3-{2-[2,5-bis(4,4,7,7-tetramethyl-4,5,6,7-tetrahydro-1-benzothiophen-2-yl)thiophen-3-yl]-3,3,4,4,5,5-hexafluorocyclopent-1-en-1-yl}-2-(4-tert-butylphenyl)-1-benzofuran-5,6-diol) (18.0 g, 0.019 mol) and potassium carbonate (21.6 g, 0.16 mol) in acetone (150 mL), 2-(2-oxooxazolidin-3-yl)ethyl 4-methylbenzenesulfonate (44.5 g, 0.16 mol) was added. The reaction mixture was heated to reflux and stirred for 16 h. Upon cooling to room temperature, the mixture was filtered through a frit and the filtrate was concentrated under reduced pressure. The product, 3,3′-(((3-(2-(2,5-bis(4,4,7,7-tetramethyl-4,5,6,7-tetrahydrobenzo[b]thiophen-2-yl)thiophen-3-yl)-3,3,4,4,5,5-hexafluorocyclopent-1-en-1-yl)-2-(4-(tert-butyl)phenyl)benzofuran-5,6-diyl)bis(oxy))bis(ethane-2,1-diyl))bis(oxazolidin-2-one) (8.1 g, 0.007 mol) was purified by column chromatography (hexanes/ethyl acetate eluent) and recrystallized in methanol/heptane to afford a yellow crystalline solid. ¹H NMR (400 MHz, CDCl₃) δ 7.25-7.21 (m, 2H), 7.20-7.14 (m, 2H), 6.92 (s, 1H), 6.91 (s, 1H), 6.69 (s, 1H), 6.37 (s, 1H), 6.18 (s, 1H), 4.36 (q, J=8.0 Hz, 4H), 4.17-4.07 (m, 4H), 3.85 (t, J=7.5 Hz, 2H), 3.73 (t, J=7.7 Hz, 4H), 3.66 (t, J=5.0 Hz, 2H), 1.69 (dd, J=18.5, 8.2 Hz, 4H), 1.53-1.43 (m, 4H), 1.31 (s, 6H), 1.21 (s, 6H), 0.97-1.15 (m, 21H).

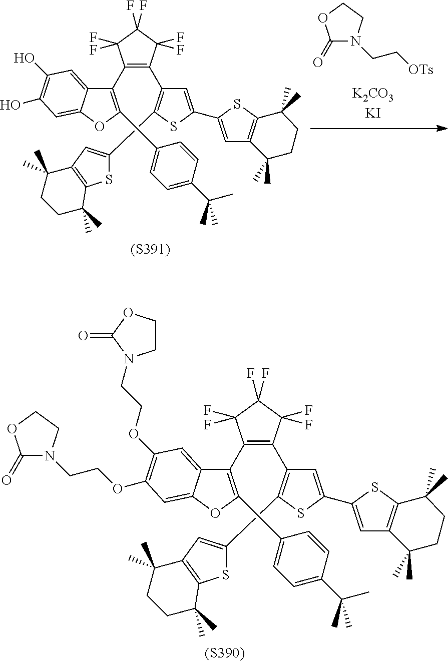

### Example 36: Preparation of Chromophore S400

A stirred solution of 3-bromo-2-(4-(tert-butyl)phenyl)-5-methoxybenzofuran (1.000 g, 2.78 mmol) in anhydrous THF (25 mL) was cooled to −78° C. n-butyllithium (1.169 ml, 2.5 M in hexanes, 2.92 mmol) was added dropwise over 30 min. The solution stirred at −78° C. for 20 mins. 2-[3′-(perfluorocyclopent-1-en-1-yl)-5-(4,4,7,7-tetramethyl-4,5,6,7-tetrahydro-1-benzothiophen-2-yl)thiophen-2-yl]-4,4,7,7-tetramethyl-4,5,6,7-tetrahydro-1-benzothiophene) (1.84 g, 2.78 mmol) in THF (10 mL) was added in one portion, the solution was warmed −35° C. and stirred for 3 h. Methanol (20 mL) was added to quench followed by concentration under reduced pressure. The product, 2-(4-(tert-butyl)phenyl)-3-(2-(5-(4,4,7,7-tetramethyl-4,5,6,7-tetrahydro-1-benzothiophen-2-yl)thiophen-2-yl]-4,4,7,7-tetramethyl-4,5,6,7-tetrahydro-1-benzothiophene))-3,3,4,4,5,5-hexafluorocyclopent-1-en-1-yl)-5-methoxybenzofuran (0.092 g, 0.1 mmol, 3.6% yield) was purified by column chromatography (hexanes/ethyl acetate eluent) and precipitated from methanol. ¹H NMR (400 MHz, CDCl₃) δ 7.22 (q, J=8.6 Hz, 4H), 6.94 (d, J=2.5 Hz, 1H), 6.81 (dd, J=8.8, 2.4 Hz, 1H), 6.68 (s, 1H), 6.41 (s, 1H), 6.12 (s, 1H), 3.82 (s, 3H), 1.74-1.63 (m, 4H), 1.48 (d, J=5.3 Hz, 4H), 1.31 (s, 6H), 1.21 (s, 6H), 0.81-1.15 (m, 21H).

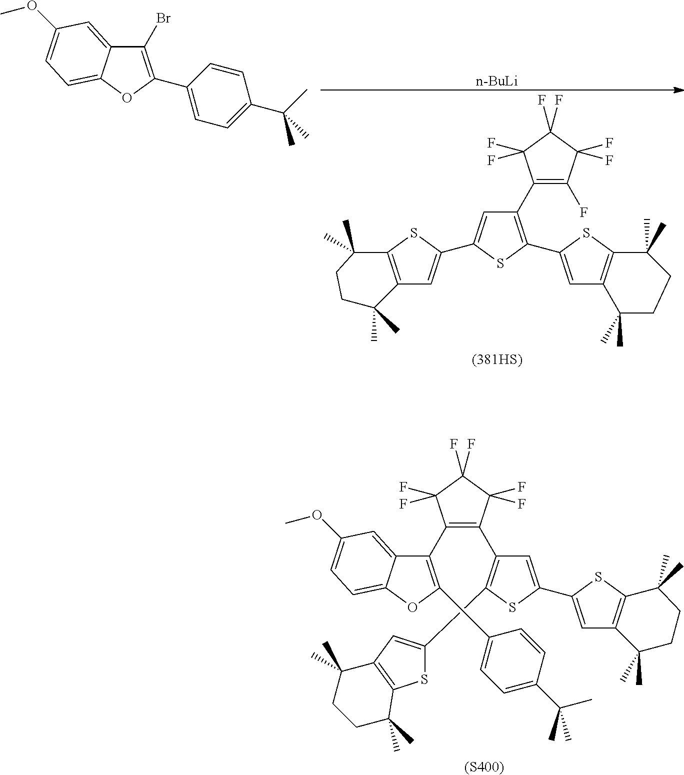

### Example 37: Preparation of Chromophore S404

To a stirred solution 3-bromo-2,5-bis({4,4,6,6-tetramethyl-4H,5H,6H-cyclopenta[b]thiophen-2-yl})thiophene (0.288 g, 0.555 mmol) in anhydrous THF (3.0 mL) at −78° C., n-butyllithium (2.5M in hexane, 0.22 mL, 0.555 mmol) was added dropwise over 5 min. The reaction mixture was stirred at this temperature for an additional 20 mins. A solution of 2-[3′-(perfluorocyclopent-1-en-1-yl)-5-(4,4,7,7-tetramethyl-4,5,6,7-tetrahydro-1-benzothiophen-2-yl)thiophen-2-yl]-4,4,7,7-tetramethyl-4,5,6,7-tetrahydro-1-benzothiophene) (0.200 g, 0.396 mmol) in anhydrous THF (5 mL) was added over 5 mins to the reaction mixture via a dropping funnel. The cold bath was removed, and the resultant mixture was warmed to room temperature over 30 mins and was stirred for an additional 2 hours. Methanol (5 mL) was added and the mixture was concentrated in vacuo. The resultant residue was dissolved in hexanes (10 mL) and washed with water (3×20 mL). The combined organic extracts were dried over sodium sulfate and concentrated in vacuo. The crude material was purified by flash chromatography using hexane:ethyl acetate (95:5) to remove the non-polar materials followed by dichloromethane:hexane (1:1) as eluent to afford S404 (83 mg, 0.090 mmol, 22.7%) as a green solid upon concentration. ¹H NMR (400 MHz, CDCl₃) δ 7.17-7.26 (m, 4H), 6.89-6.96 (m, 2H)), 6.57 (s, 1H), 6.25 (s, 1H), 3.87 (s, 3H), 3.87 (s, 3H), 1.91 (s, 2H), 1.36 (s, 6H), 1.29 (s, 6H), 1.00-1.19 (m, 21H).

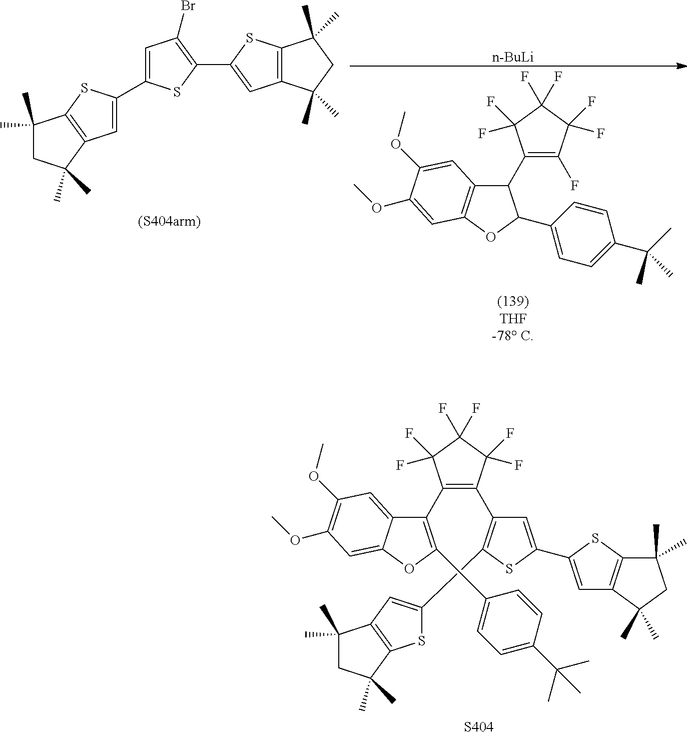

### Example 38: Preparation of Chromophore S405

To a stirred solution 3-bromo-2,5-bis({4,4,8,8-tetramethyl-4H,5H,6H,7H,8H-cyclohepta[b]thiophen-2-yl})thiophene (1.541 g, 0.003 mol) in anhydrous THF (25.0 mL) at −78° C., n-butyllithium (2.5M in hexane, 1.1 mL, 0.003 mol) was added dropwise over 20 mins. The reaction mixture was stirred at this temperature for an additional 20 mins. A solution of 2-[3′-(perfluorocyclopent-1-en-1-yl)-5-(4,4,7,7-tetramethyl-4,5,6,7-tetrahydro-1-benzothiophen-2-yl)thiophen-2-yl]-4,4,7,7-tetramethyl-4,5,6,7-tetrahydro-1-benzothiophene) (1.000 g, 0.002 mol) in anhydrous THF (16.7 mL) was added over 5 mins to the reaction mixture via a dropping funnel. The cold bath was removed, and the resultant mixture warmed to room temperature over 30 mins and was stirred for an additional 2 hours. Methanol (5 mL) was added and the mixture was concentrated in vacuo. The resultant residue was dissolved in hexanes (50 mL) and washed with water (3×200 mL). The combined organic extracts were dried over sodium sulfate and concentrated in vacuo. The crude material was purified by flash chromatography using hexane:ethyl acetate (9:1) as eluent and precipitated from methanol to afford S405 (0.521 g, 0.001 mol, 26.8%) as a yellow powder. ¹H NMR (400 MHz, CDCl₃) δ 7.14-7.25 (m, 4H), 6.95 (m, 2H), 6.81 (s, 1H), 6.51 (s, 1H), 6.21 (s, 1H), 3.91 (m, 6H), 1.85-1.92 (m, 2H), 1.68-1.79 (m, 6H), 1.42 (s, 6H), 1.31 (s, 6H), 1.11-1.22 (m, 12H), 1.08 (s, 9H).

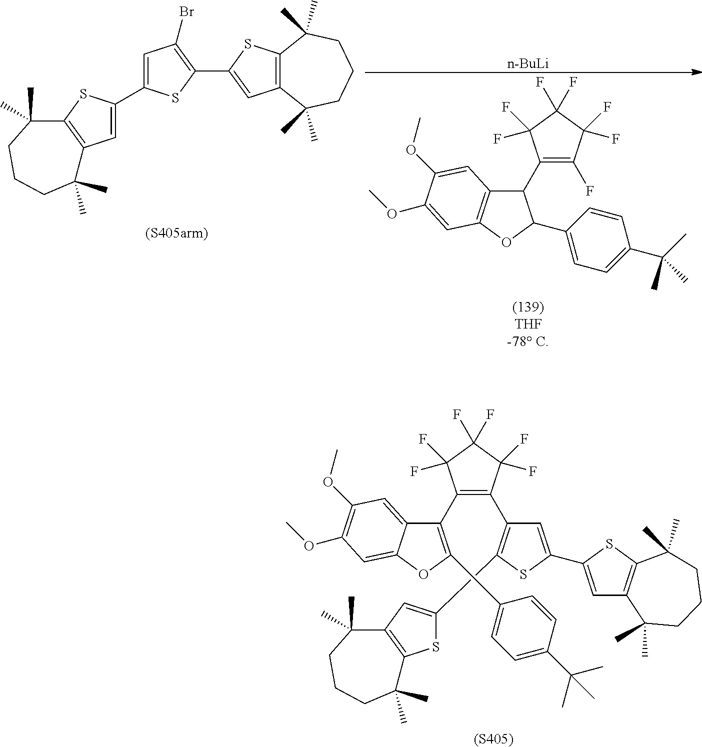

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1-24. (canceled)

25. A compound according to Formula 1A/1B, reversibly convertible under photochromic and electrochromic conditions between a ring-open isomer A and a ring-closed isomer B: 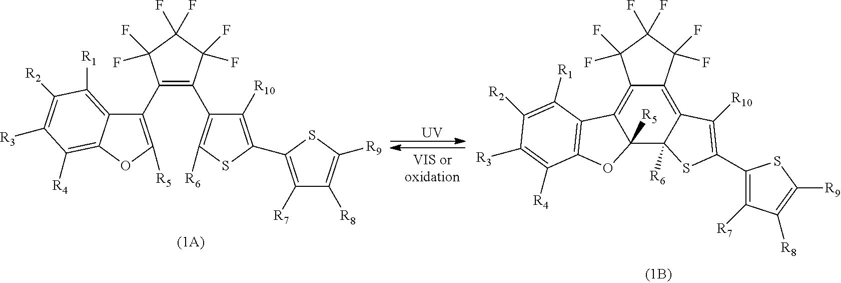
wherein:
each of R₁, R₂, R₃, and R₄ is independently:
H,
a linear or branched, saturated or unsaturated, substituted or unsubstituted alkyl group with 1 to 20 carbons,
a linear or branched, saturated or unsaturated, substituted or unsubstituted heteroalkyl group with 1 to 20 carbons and comprising one or more of O, S, N or Si, or
—O—R, wherein R is
a linear or branched, saturated or unsaturated, substituted or
unsubstituted alkyl group with 1 to 20 carbons, or
a linear or branched, saturated or unsaturated, substituted or
unsubstituted heteroalkyl group with 1 to 20 carbons and comprising one or more of O, S, N or Si;
R₅ is 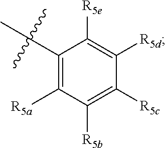
R₆ is 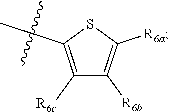
each of R₅ₐ, R_{5b}, R_{5c}, R_{5d}, and R₅ₑ is independently:
H,
a linear or branched, saturated or unsaturated, substituted or unsubstituted alkyl group with 1 to 20 carbons,
a linear or branched, saturated or unsaturated, substituted or unsubstituted heteroalkyl group with 1 to 20 carbons and comprising one or more of O, S, N or Si, or
—O—R, wherein R is
a linear or branched, saturated or unsaturated, substituted or unsubstituted alkyl group with 1 to 20 carbons, or
a linear or branched, saturated or unsaturated, substituted or unsubstituted heteroalkyl group with 1 to 20 carbons and comprising one or more of O, S, N or Si;
each of R₆ₐ, R_{6b} and R_{6c} is independently:
H,
a linear or branched, saturated or unsaturated, substituted or unsubstituted alkyl group with 1 to 20 carbons,
a linear or branched, saturated or unsaturated, substituted or unsubstituted heteroalkyl group with 1 to 20 carbons and comprising one or more of O, S, N or Si,
wherein R_{6b} is of equal or larger steric size than R₆ₐ, or
R₆ₐ and R_{6b} are both —C(R₁₂)(R₁₃)— and joined by —(C(R₁₄)(R₁₅))ₙ— to form a 5-, 6- or 7-membered ring where n is 1, 2 or 3, respectively, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si;
each of R₇, R₈ and R₉ is independently:
H,
a linear or branched, saturated or unsaturated, substituted or unsubstituted alkyl group with 1 to 20 carbons,
a linear or branched, saturated or unsaturated, substituted or unsubstituted heteroalkyl group with 1 to 20 carbons and comprising one or more of O, S, N or Si, or
R₇ and R₈ or R₈ and R₉ are both —C(R₁₆)(R₁₇)— and joined by —(C(R₁₈)(R₁₉))ₙ— to form a 5-, 6- or 7-membered ring where n is 1, 2 or 3, respectively, wherein each or R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si; and
R₁₀ is H or a linear or branched, saturated or unsaturated, substituted or unsubstituted alkyl group with 1 to 20 carbons.

26. The compound of claim 25, wherein R_{6b} is not H.

27. The compound of claim 25, wherein R₆ₐ and R_{6c} are H and R_{6b} is methyl, ethyl, propyl, butyl, pentyl or hexyl.

28. The compound of claim 25, wherein R_{6b} is tert-butyl.

29. The compound of claim 25, wherein R₆ₐ and R_{6b} are each —C(R₁₂)(R₁₃)— and joined by —(C(R₁₄)(R₁₅))₂— to form a 6-membered ring, wherein each of R₁₂, R₁₃, R₁₄ and R₁₅ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si.

30. The compound of claim 29, wherein each of R₁₂ and R₁₃ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si and R₁₄ and R₁₅ are H.

31. The compound of claim 30, wherein each of R₁₂ and R₁₃ is independently methyl, ethyl, propyl or butyl and R₁₄ and R₁₅ are H.

32. The compound of claim 31, wherein R₁₂ and R₁₃ are methyl and R₁₄ and R₁₅ are H.

33. The compound of claim 25, wherein R₅ₐ, R_{5b}, R_{5d} and R₅ₑ are H and R_{5c} is methyl, ethyl, propyl, butyl, pentyl or hexyl.

34. The compound of claim 33, wherein R_{5c} is tert-butyl.

35. The compound of claim 25, wherein R₁ and R₄ are H and R₂ and R₃ are a linear or branched, saturated or unsaturated, substituted or unsubstituted heteroalkyl group with 1 to 8 carbons and comprising one or more of O or N.

36. The compound of claim 35, wherein R₂ and R₃ are independently an ester, an ether, a carbamate or an imide.

37. The compound of claim 25, wherein R₂ and R₃ are —OCH₃, —OCOCH₃, —O(CH₂)₃CO₂CH₂CH₃, —O(CH₂)₃OCH₃,

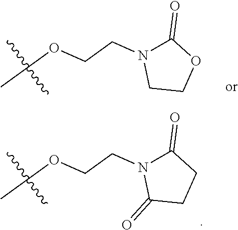

38. The compound of claim 25, wherein R₇ and R₉ are H and R₈ is methyl, ethyl, propyl, butyl, pentyl or hexyl.

39. The compound of claim 25, wherein R₈ is tert-butyl.

40. The compound of claim 25 wherein R₈ and R₉ are each —C(R₁₆)(R₁₇)— and joined by —(C(R₁₈)(R₁₉))₂— to form a 6-membered ring, wherein each of R₁₆, R₁₇, R₁₈ and R₁₉ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si.

41. The compound of claim 40, wherein each of R₁₆ and R₁₇ is independently H or a linear or branched, saturated or unsaturated, substituted or unsubstituted alkyl group with 1 to 20 carbons, or a linear or branched, saturated or unsaturated, substituted or unsubstituted heteroalkyl group with 1 to 20 carbons and comprising one or more of O, S, N or Si and R₁₈ and R₁₉ are H.

42. The compound of claim 41, wherein each of R₁₆ and R₁₇ is independently methyl, ethyl, propyl or butyl and R₁₈ and R₁₉ are H.

43. The compound of claim 42, wherein R₁₆ and R₁₇ are methyl and R₁₈ and R₁₉ are H.

44. The compound of claim 25, selected from the group consisting of:

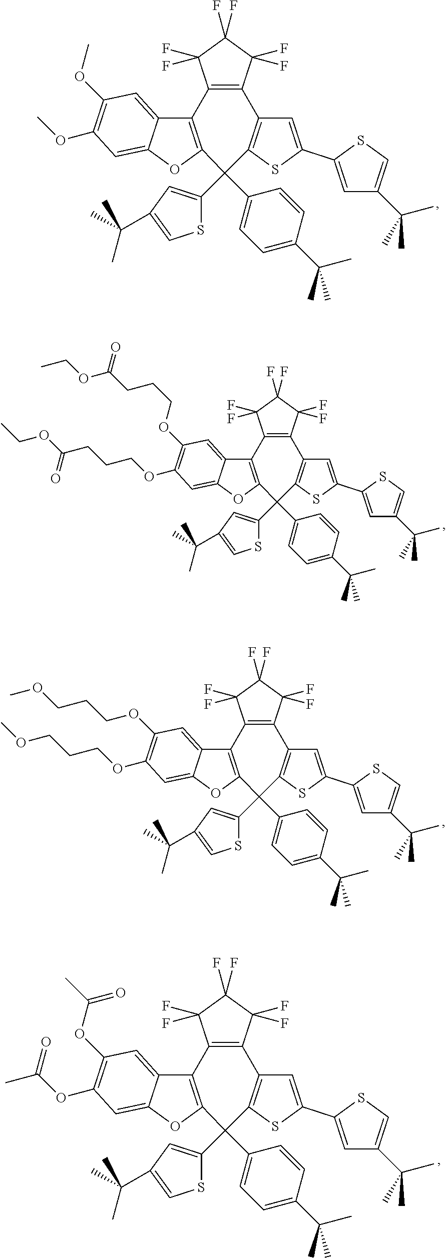

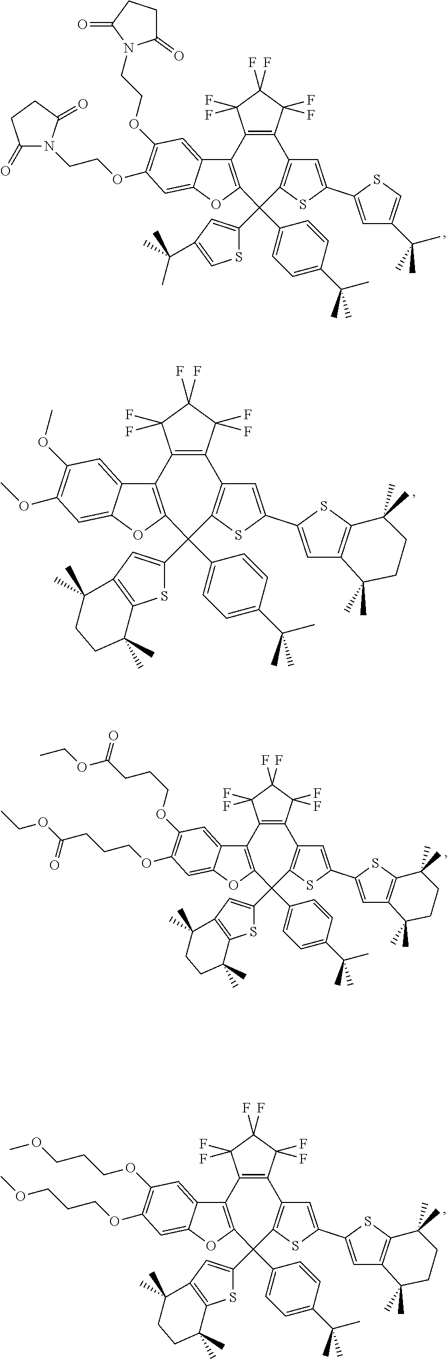

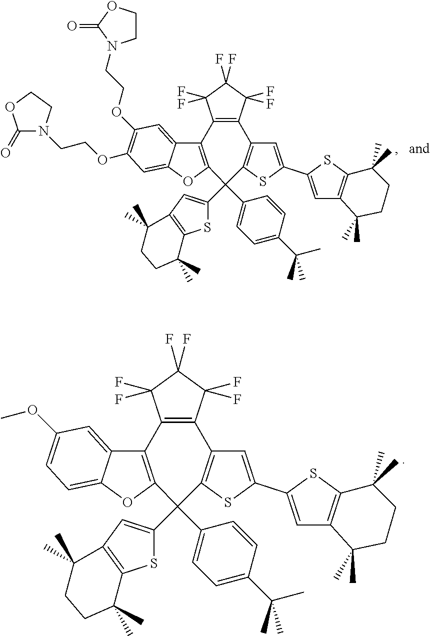

45-57. (canceled)
